(19) United States
(12) Patent Application Publication
(10) Pub. No.: US 2025/0297224 A1
(43) Pub. Date: Sep. 25, 2025

(54) CARDIOMYOCYTES AND COMPOSITIONS AND METHODS FOR PRODUCING THE SAME

(71) Applicants: President and Fellows of Harvard College, Cambridge, MA (US); The Children's Medical Center Corporation, Boston, MA (US)

(72) Inventors: Jia Liu, Cambridge, MA (US); Junya Aoyama, Cambridge, MA (US); Qiang Li, Cambridge, MA (US); Ren Liu, Cambridge, MA (US); Richard T. Lee, Cambridge, MA (US); Jessica Garbern, Brookline, MA (US)

(21) Appl. No.: 18/709,798

(22) PCT Filed: Nov. 11, 2022
(86) PCT No.: PCT/US2022/049741

§ 371 Date: May 13, 2024

Related U.S. Application Data
(60) Provisional application No. 63/278,884, filed on Nov. 12, 2021.

(51) Int. Cl. C12N 5/077; A61K 35/34

(52) U.S. Cl. CPC C12N 5/0657; A61K 35/34; C12N 2501/999; C12N 2502/28; C12N 2506/45; C12N 2513/00

(57) ABSTRACT

Disclosed herein are methods for generating mature cardiomyocytes and compositions including mature cardiomyocytes. Also disclosed herein are methods for enhancing electrophysiological maturation of cardiomyocytes.

### RELATED APPLICATIONS

This application claims the benefit of and priority to U.S. Provisional Application No. 63/278,884, filed on Nov. 12, 2021. The entire teachings of the above application are incorporated herein by reference.

### GOVERNMENT SUPPORT

This invention was made with government support under grants HL150335 and HL151684 awarded by the National Institutes of Health (NIH), and under grant 2038603 awarded by the National Science Foundation (NSF). The government has certain rights in the invention.

### BACKGROUND OF THE INVENTION

Stem cell approaches to treat chronic heart failure will require production of ventricular cardiomyocytes to improve systolic heart function and reduce the incidence of ventricular arrhythmias. However, cardiomyocytes derived from embryonic or induced pluripotent stem cells (ESCs or iPSCs, respectively) using current differentiation protocols remain functionally immature. These immature cardiomyocytes display automaticity or pacemaker-like activity which results in potentially life-threatening ventricular arrhythmias when delivered to adult animal models and also have a less organized sarcomere structure preventing adequate contractile force. Successful translation of stem cell-derived therapies for treatment of cardiovascular disease will require developing improved methods for maturation of stem cell-derived cardiomyocytes.

To develop a replacement therapy using allogeneic human pluripotent stem cell-derived cardiomyocytes (PSC-CMs) as a viable therapeutic option, methods to culture large numbers of cardiomyocytes with good manufacturing practices for off-the-shelf use must be developed. Maintenance of 2D cultures is labor-intensive with significant batch-to-batch variability; in contrast, maintenance and differentiation of PSCs in three-dimensional (3D) bioreactor systems is more amenable to scale up, reduces labor time, and small volume sampling allows for improved quality control. However, this shift from 2D to 3D culture alters the phenotype of the cells due to differential regulation of various signaling pathways in different culture geometries. The field has yet to devise a uniform protocol that efficiently produces mature cardiomyocytes in 3D.

### SUMMARY OF THE INVENTION

There is a need for methods or protocols for the generation of mature cardiomyocytes for use in cell therapy and screening, among other uses. In some embodiments, by co-culturing cardiomyocytes with endothelial cells, maturation of the cardiomyocytes may be enhanced. In some embodiments, by regulating FOXO-FOXM signaling, maturation of the cardiomyocytes may be enhanced.

Disclosed herein are methods of producing human mature cardiomyocytes comprising co-culturing a human immature cardiomyocyte with a human endothelial cell. In some embodiments, the endothelial cell comprises an iPSC-derived endothelial cell and/or the immature cardiomyocyte comprises an iPSC-derived immature cardiomyocyte.

Also disclosed herein are methods of producing a human mature cardiomyocyte comprising co-culturing an iPSC-derived immature cardiomyocyte with an iPSC-derived endothelial cell. In some embodiments, the iPSC-derived immature cardiomyocyte comprises a human cardiomyocyte and/or the iPSC-derived endothelial cell comprises a human endothelial cell.

In some embodiments, the methods disclosed herein further comprise culturing the iPSC-derived cardiomyocyte and the iPSC-derived endothelial cell with at least one cardiomyocyte maturation factor. In some embodiments, the at least one cardiomyocyte maturation factor is selected from the group consisting of an mTOR signaling pathway inhibitor, a p53 upregulator, a FOXO activator, a FOXM1 inhibitor, and combinations thereof. In some embodiment, the at least one cardiomyocyte maturation factor is selected from the group consisting of nutlin-3a, quercetin, Torin1, Torin2, LOM612, metformin, resveratrol, Selinexor, celecoxib, doxorubicin, carbenoxolone, RCM1, FDI-6, thiostrepton, honokiol, siomycin A, curcumin, bortezomib, MG115, MG132, and combinations thereof. In some embodiments, the at least one cardiomyocyte maturation factor is selected from the group consisting of nutlin-3a, quercetin, Torin1. Torin2, LOM612, RCM1, and combinations thereof.

In some embodiments, the mature cardiomyocyte exhibits increased expression of a marker selected from the group consisting of cardiac troponin T (TNNT2), cardiac troponin I (TNNI3), Kir2.1, connexin 43 (Cx43), CD36, and combinations thereof as compared to an immature cardiomyocyte. In some embodiments, the mature cardiomyocyte exhibits increased expression of cardiac troponin T (TNNT2), cardiac troponin I (TNNI3), Kir2.1. connexin 43 (Cx43), and/or CD36 as compared to an immature cardiomyocyte. In some embodiments, the mature cardiomyocyte exhibits increased respiratory reserve capacity as compared to an immature cardiomyocyte.

In some embodiments, the mature cardiomyocyte exhibits decreased activity of an action selected from the group consisting of beating rate, automaticity, and combinations thereof as compared to an immature cardiomyocyte. In some embodiments, the mature cardiomyocyte exhibits a decreased beating rate and/or decreased automaticity as compared to an immature cardiomyocyte. In some embodiments, the mature cardiomyocyte exhibits a spontaneous beating rate of less than 3 beats per minute. In some embodiments, the mature cardiomyocyte is an electrically mature cardiomyocyte. In some embodiments, the mature cardiomyocyte exhibits a decreased risk of arrhythmia after delivery in vivo as compared to an immature cardiomyocyte. In some embodiments, the mature cardiomyocyte exhibits improved engraftment in vivo as compared to an immature cardiomyocyte.

In some embodiments, the co-culturing occurs in three-dimensional culture. In some embodiments, the co-culturing occurs in vitro. In some embodiments, the co-culturing occurs in vivo.

Disclosed herein are non-naturally occurring cardiomyocytes produced by the methods disclosed herein. In some embodiments, the non-naturally occurring cardiomyocyte exhibits increased expression of a marker selected from the group consisting of TNNT2. TNNI3, Kir2.1. Cx43, CD36, and combinations thereof as compared to an immature cardiomyocyte. In some embodiments, the non-naturally occurring cardiomyocyte exhibits increased respiratory reserve capacity as compared to an immature cardiomyocyte. In some embodiments, the non-naturally occurring cardiomyocyte exhibits decreased activity of an action selected from the group consisting of beating rate, automaticity, and combinations thereof as compared to an immature cardiomyocyte. In some embodiments, the non-naturally occurring cardiomyocyte exhibits a decreased beating rate and/or decreased automaticity as compared to an immature cardiomyocyte. In some embodiments, the non-naturally occurring cardiomyocyte exhibits a spontaneous beating rate of less than 3 beats per minute. In some embodiments, the non-naturally occurring cardiomyocyte is an electrically mature cardiomyocyte.

Disclosed herein are methods of producing a mature cardiomyocyte from an immature cardiomyocyte comprising contacting the immature cardiomyocyte with at least one cardiomyocyte maturation factor selected from the group consisting of FOXO activator, FOXM1 inhibitor, and combinations thereof.

In some embodiments, the at least one cardiomyocyte maturation factor is selected from the group consisting of Torin2, LOM612, metformin, resveratrol, Selinexor, celecoxib, doxorubicin, carbenoxolone, RCM1, FDI-6, thiostrepton, honokiol, siomycin A, curcumin, bortezomib, MG115, MG132, and combinations thereof. In some embodiments, the at least one cardiomyocyte maturation factor is selected from the group consisting of LOM612. RCM1, and combinations thereof. In some embodiments, the at least one cardiomyocyte maturation factor comprises LOM612 and/or RCM1.

In some embodiments, the immature cardiomyocyte is contacted with at least one additional maturation factor selected from the group consisting of mTOR signaling pathway inhibitor, a p53 upregulator, and combinations thereof. In some embodiments, the at least one additional maturation factor is selected from the group consisting of nutlin-3a, quercetin, Torin1, and combinations thereof.

In some embodiments, the mature cardiomyocyte exhibits increased expression of cardiac troponin T (TNNT2), cardiac troponin I (TNNI3), Kir2.1, and combinations thereof as compared to an immature cardiomyocyte. In some embodiments, the mature cardiomyocyte exhibits increased expression of cardiac troponin T (TNNT2), cardiac troponin I (TNNI3) and/or Kir2.1 as compared to an immature cardiomyocyte.

In some embodiments, the mature cardiomyocyte exhibits increased activity of an action selected from the group consisting of mean beat amplitude, upstroke velocity, maximum oxygen consumption rate (OCR), respiratory reserve capacity, and combinations thereof as compared to an immature cardiomyocyte. In some embodiments, the mature cardiomyocyte exhibits increased mean beat amplitude, upstroke velocity, maximum oxygen consumption rate, and/or respiratory reserve capacity as compared to an immature cardiomyocyte.

In some embodiments, the mature cardiomyocyte is an electrically mature cardiomyocyte and/or a metabolically mature cardiomyocyte.

In some embodiments, the mature cardiomyocyte exhibits a decreased risk of arrhythmia after delivery in vivo as compared to an immature cardiomyocyte. In some embodiments, the mature cardiomyocyte exhibits improved engraftment in vivo as compared to an immature cardiomyocyte.

In some embodiments, the co-culturing occurs in three-dimensional culture. In some embodiments, the co-culturing occurs in vitro. In some embodiments, the co-culturing occurs in vivo.

Also disclosed herein are non-naturally occurring cardiomyocyte produced by the methods disclosed herein. In some embodiments, the non-naturally occurring cardiomyocyte exhibits increased expression of a marker selected from the group consisting of TNNT2, TNNI3, Kir2.1, and combinations thereof as compared to an immature cardiomyocyte. In some embodiments, the non-naturally occurring cardiomyocyte exhibits increased activity of an action selected from the group consisting of mean beat amplitude, upstroke velocity, maximum oxygen consumption rate (OCR), respiratory reserve capacity, and combinations thereof as compared to an immature cardiomyocyte. In some embodiments, the non-naturally occurring cardiomyocyte is an electrically mature cardiomyocyte and/or a metabolically mature cardiomyocyte.

Also disclosed herein are methods of treatment comprising administering to a subject in need thereof a composition comprising at least one mature cardiomyocyte produced by the methods disclosed herein. In some embodiments, the subject has, or is at risk of developing, a ventricular arrhythmia, decreased systolic heart function, chronic heart failure, congenital heart disease, or other heart disease.

Also disclosed herein are uses of a composition in the manufacture of a medicament for treatment of a heart condition, wherein the treatment comprises administration of the medicament to a subject in need thereof, wherein the composition comprises at least one mature cardiomyocyte produced by the methods disclosed herein. In some embodiments, the subject has, or is at risk of developing, a ventricular arrhythmia, decreased systolic heart function, chronic heart failure, congenital heart disease, or other heart disease.

Also disclosed herein are three-dimensional structure comprising the mature cardiomyocytes produced by the methods disclosed herein. In some embodiments, the three-dimensional structure is a matrix or scaffold. In some embodiments, the three-dimensional structure is administered to a subject.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies-A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, NJ, 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, MD) and National Center for Biotechnology Information, National Library of Medicine (Bethesda, MD), as of May 1, 2010, World Wide Web URL: ncbi.nlm.nih.gov/omim/and in Online Mendelian Inheritance in Animals (OMIA), a database of genes, inherited disorders and traits in animal species (other than human and mouse), at omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

### BRIEF DESCRIPTION OF THE DRAWINGS

The patent or application file contains at least one drawing executed in color. Copies of this patent or patent application publication with color drawings will be provided by the Office upon request and payment of the necessary fec.

FIG. 1 provides a schematic demonstrating that inadequate maturation of cardiomyocytes is a barrier to clinical translation. For example, the delivery of immature stem cell-derived cardiomyocytes to large animal models may lead to potentially life threatening ventricular arrhythmias. However, after delivery of immature cardiomyocytes to animals, the cardiomyocytes undergo further maturation, suggesting that other cells may interact with cardiomyocytes to promote their maturation.

FIG. 2 provides a schematic demonstrating the potential interaction of co-culturing endothelial cells with cardiomyocytes and some predicted benefits of the co-culture.

FIGS. 3A-3B demonstrate exemplary differentiation protocols for obtaining cardiomyocytes (FIG. 3A) (see Lian et al. Nat Protoc 2013; 8 (1): 162-75) and endothelial cells (FIG. 3B) (see Patsch et al. Nat Cell Biol 2015; 17 (8): 994-1003).

FIGS. 4A-4B demonstrate increased expression of selected cardiomyocyte markers TNNT2 and CD144 (FIG. 4A) and TNNI3, Kir2.1, Cx43, and CD36 (FIG. 4B) when cardiomyocytes are co-cultured with endothelial cells at varying ratios, compared to when cardiomyocytes are cultured without endothelial cells.

FIGS. 5A-5E demonstrate that co-culture with endothelial cells increases cardiomyocyte expression of Kir 2.1 and connexin 43. Endothelial cell co-culture leads to increased expression of selected cardiac markers in a dose dependent manner by flow cytometry analysis: TNNT2 (FIG. 5A), TNNI3 (FIG. 5B), Kir2.1 (FIG. 5C), Connexin 43 (FIG. 5D), and CD36 (FIG. 5E).

FIGS. 6A-6B demonstrate that cardiomyocytes cultured on mesh nanoelectronics device with endothelial cells showed accelerated maturation of electrical profile and reduced beating rate. FIG. 6A provides electrical recordings from distinct electrodes within the device on days 15, 18, 23, and 30 after initiation of iPSC-CM differentiation. iPSC-CMs alone (CM) have wider action potential duration with a faster intrinsic beating rate compared to co-cultured conditions (CM+EC). FIG. 6B provides electrical recordings from individual channels over a shorter time scale to visualize differences in electrical profiles between CM vs CM+EC. CM only has a wide complex suggestive of slow depolarization and repolarization, while CM+EC cells have a rapid depolarization depicted by a sharp spike at onset of each beat. These results demonstrate the electrical profile of iPSC-CMs co-cultured with iPSC-ECs have a more mature electrical profile suggesting enhanced electrical maturation.

FIGS. 7A-7B demonstrate electrically immature cardiomyocytes using surface markers. FIG. 7A shows single cell RNAseq performed to reveal differences in gene expression in cardiomyocytes (CMs) co-cultured with endothelial cells (ECs) (EM+EC) versus CM alone. FIG. 7B shows differential expression of potential cardiomyocyte surface markers for CM+EC vs CM only.

FIGS. 8A-8C demonstrate that cardiomyocytes that were VCAM1-negative at the time of sorting had increased expression of Kir2.1 (FIG. 8A), CD36 (FIG. 8B), and connexin 43 (FIG. 8C). Cardiomyocytes were sorted with the VCAM-1 marker and replaced as positive VCAM-1 or negative VCAM1, after 5 days they were analyzed with FACS.

FIGS. 9A-9D demonstrate co-culture of iPSC-CMs and iPSC-ECs. FIGS. 9A-9B show the interface between iPSC-CMs and iPSC-ECs immunostained with TNNT2 (red), DAPI (blue) and CD31 (green) (FIG. 9A) or Cx43 (green) (FIG. 9B). FIG. 9C shows that a combination of iPSC-ECs and iPSC-CMs increases the percentage of live cells compared to either iPSC-ECs or iPSC-CMs alone. FIG. 9D provides western analysis of iPSC-CMs alone, iPS-CMs+iPSC-ECs, or iPSC-ECs alone. These results suggest that co-culture of iPSC-CMs+iPSC-ECs increases survival and maturation versus iPSC-CMs alone.

FIG. 10 provides in vivo data showing vessel like structures 7 days after intramyocardial injection of iPSC-ECs into an athymic rat. Vessel-like structures stain positive for CD32 and human specific Ulex Europaeus agglutinin 1 (UEA 1) but not TNNT2, suggesting that iPSC-ECs formed microvessels within the rat myocardium within 1 week after delivery.

FIG. 11 shows resting membrane potential in Torin1-treated (200 nM) iPSC-CMs in 2D is decreased compared to control (DMSO).

FIGS. 12A-12H demonstrate that Nutlin-3a but not Torin1 treatment of iPSC-CMs increases expression of TNNT2, Kir2.1, and p53 in 3D culture. Nutlin-3a increases the percentage (FIG. 12A) and mean fluorescence intensity (MFI) (FIG. 12B) of TNNT2 in iPSC-CMs as well as the percentage (FIG. 12C) and MFI (FIG. 12D) of Kir2.1 in TNNT2+iPSC-CMs by flow cytometry. Nutlin-3a increases the percentage (FIG. 12E) and MFI (FIG. 12F) of p53 in TNNT2+iPSC-CMs. Nutlin-3a increases mean beat amplitude (FIG. 12G) and mean spike amplitude (FIG. 12H) of iPSC-CMs as quantified using a multielectrode array. *p<0.05. **p<0.01. ***p<0.001. ****p<0.0001 by one-way ANOVA. iPSC-CMs differentiated and treated in 3D.

FIGS. 13A-13D demonstrate that quercetin increases expression of Kir2.1 Quercetin increases % TNNT2+iPSC-CMs expressing % of Kir2.1 (FIG. 13A), mean fluorescence intensity (MFI) of Kir2.1 (FIG. 13B), % of p53+TNNT2+iPSC-CMs (FIG. 13C), and MFI of p53 by flow cytometry (FIG. 13D). iPS in 3D and treated in 3D.

FIGS. 14A-14D demonstrate that inhibition of FOXM1 with RCM1 increases expression of Kir2.1. Inhibition of FOXM1 with RCM1 increases % TNNT2+ (FIG. 14A) but not mean fluorescence intensity (MFI) of TNNT2 (FIG. 14B). RCM1 increases % TNT2+iPSC-CMs expressing Kir2.1 (FIG. 14C) and mean fluorescence intensity (MFI) of Kir2.1 (FIG. 14D).

FIG. 15 provides diagrams demonstrating the differences between immature cardiomyocytes and mature cardiomyocytes.

FIGS. 16A-16C provide methods for obtaining and characterizing cardiomyocytes. FIG. 16A provides a differentiation protocol for obtaining cardiomyocytes (see Lian et al. Nat Protoc 2013; 8 (1): 162-75). FIG. 16B identifies the activators and inhibitors used during the differentiation protocol outlined in FIG. 16A. FIG. 16C outlines methods that may be used to characterize cardiomyocytes.

FIGS. 17A-17C demonstrate that contractile properties improve with FOXO activation. FIGS. 17A-17B shows FOXO activation with LOM612 or FOXM1 inhibition with RCM-1 increases protein expression of TNNT2 and TNNI3 and FOXO inhibition with AS1842856 decreases protein expression of TNNT2 and TNNI3. FIG. 17C shows FOXO activation with LOM612 increases mean beat amplitude by multielectrode array analysis.

FIGS. 18A-18C demonstrate that FOXO activation promotes metabolic maturation of PSC-CMs. FOXO activation with LOM612 (FIG. 18A) or FOXM1 inhibition with RCM-1 (FIG. 18B) increases maximum oxygen consumption rate (OCR) and respiratory reserve capacity, while inhibition of FOXO with AS1842856 (FIG. 18C) inhibits maximum OCR and respiratory reserve capacity.

FIGS. 19A-19C demonstrate FOXO activation improves electrophysiological properties of PSC-CMs. FOXO activation with LOM612 enhances protein (FIG. 19B) but not RNA expression of Kir2.1 (FIG. 19A). FOXO activation increases the upstroke velocity (spike slope) (FIG. 19C).

FIG. 20 demonstrates that FOXO inhibition with AS1842856 reduces cardiac marker RNA levels.

### DETAILED DESCRIPTION OF THE INVENTION

Current differentiation protocols to produce cardiomyocytes from human induced pluripotent stem cells (iPSCs) are capable of generating highly pure cardiomyocyte populations as determined by expression of cardiac troponin T. However, these cardiomyocytes remain immature, more closely resembling the fetal state, with a lower maximum contractile force, slower upstroke velocity, and immature mitochondrial function compared with adult cardiomyocytes. Immaturity of iPSC-derived cardiomyocytes may be a significant barrier to clinical translation of cardiomyocyte cell therapies for heart disease.

Aspects of the disclosure relate to compositions, methods, kits, and agents for generating cardiomyocytes (referred to herein as non-naturally occurring cardiomyocytes, non-native cardiomyocytes, quiescent cardiomyocytes, or mature cardiomyocytes) from at least one immature cardiomyocyte (e.g., an immature cardiomyocyte made from a stem cell), and mature cardiomyocytes produced by those compositions, methods, kits, and agents for use in cell therapies, assays, and various methods of treatment.

The in vitro-produced cardiomyocytes generated according to the methods described herein demonstrate many advantages; for example, they are electrically mature (e.g., exhibit decreased automaticity), contractility mature, and metabolically mature. In addition, the generated cardiomyocytes may provide a new platform for cell therapy (e.g., transplantation into a subject in need of additional and/or functional cardiomyocytes) and research.

### Definitions

For convenience, certain terms employed herein, in the specification, examples and appended claims are collected here. Unless otherwise defined, all technical and scientific terms used herein have the same meaning as commonly understood by one of ordinary skill in the art to which this invention belongs.

As used herein, the term “somatic cell” refers to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body-apart from the sperm and ova, the cells from which they are made (gametocytes) and undifferentiated stem cells-is a somatic cell type: internal organs, skin, bones, blood, and connective tissue are all made up of somatic cells. In some embodiments the somatic cell is a “non-embryonic somatic cell,” by which is meant a somatic cell that is not present in or obtained from an embryo and does not result from proliferation of such a cell in vitro. In some embodiments the somatic cell is an “adult somatic cell,” by which is meant a cell that is present in or obtained from an organism other than an embryo or a fetus or results from proliferation of such a cell in vitro.

As used herein, the term “adult cell” refers to a cell found throughout the body after embryonic development.

The term “progenitor” or “precursor” cell are used interchangeably herein and refer to cells that have a cellular phenotype that is more primitive (i.e., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “phenotype” refers to one or a number of total biological characteristics that define the cell or organism under a particular set of environmental conditions and factors, regardless of the actual genotype.

The term “pluripotent” as used herein refers to a cell with the capacity to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g., iPS cells as that term is defined herein) also have the characteristic of the capacity of extended passaging without loss of growth potential, relative to primary cell parents, which generally have capacity for only a limited number of divisions in culture.

As used herein, the terms “iPS cell” and “induced pluripotent stem cell” are used interchangeably and refer to a pluripotent stem cell artificially derived (e.g., induced or by complete reversal) from a non-pluripotent cell, typically an adult somatic cell, for example, by inducing a forced expression of one or more genes.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art. As used herein, the term “pluripotent stem cell” includes embryonic stem cells, induced pluripotent stem cells, placental stem cells, etc.

As used herein “quiescence” or “cellular quiescence” is used to refer to a cellular resting state triggered by nutrient deprivation and is characterized by the ability to re-enter the cell cycle in response to appropriate stimuli. Quiescent cells retain metabolic and transcriptional activity. Cells can have varying depths of quiescence, including a transitional entry period into G₀, deep G₀, and a Gₐₗₑₙₜ state, which is a more shallow state of quiescence during which cells are more responsive to stimuli triggering return to the cell cycle. Quiescent cardiomyocytes may exhibit expression of one or more quiescence markers, including p16 and p130.

The terms “endogenous cardiomyocyte” or “endogenous mature cardiomyocyte” are used herein to refer to a mature cardiomyocyte. A mature cardiomyocyte may exhibit electrical maturity, contractile maturity, and/or metabolic maturity. The phenotype of a cardiomyocyte is well known by persons of ordinary skill in the art, and includes, for example, ability to spontaneously beat, expression of markers such as cardiac troponin, TNNT2, TNNI3, myosin heavy chain, MYH6, MYH7, ryanodine receptor (RyR), sodium channel protein SCN5a, potassium voltage-gated channel KCNJ2, ATP2A2, PPARGC1a, Cx43, as well as distinct morphological characteristics such as organized sarcomeres, having rod shaped cells, and having T-tubules.

As used herein “cardiomyocyte.” “non-naturally occurring cardiomyocyte,” “non-native cardiomyocyte,” “quiescent cardiomyocyte.” and “mature cardiomyocyte.” all refer to cardiomyocytes produced by the methods as disclosed herein. The cardiomyocytes may be ventricular-, atrial-, and/or nodal-type cardiomyocytes, or a mixed population of cardiomyocytes. Cardiomyocytes may exhibit one or more features which may be shared with endogenous cardiomyocytes, including, but not limited to, capacity to beat spontaneously, are electrically mature, metabolically mature, contractility mature, exhibit appropriate expression of one or more gene markers (e.g., TNNI3, TNNT2, Kir2.1, Cx43, and CD36), exhibit appropriate expression of one or more quiescence markers (e.g., p16 and p130), exhibit appropriate morphological characteristics (e.g., rod shaped cells and organized sarcomeres), and expandability in culture. However non-naturally occurring cardiomyocytes are not identical to and are distinguishable from endogenous cardiomyocytes as described herein, including distinction on the basis of gene expression. For example, non-naturally occurring cardiomyocytes may express similar proteins but at distinguishable expression levels as compared to endogenous cardiomyocytes.

The term “cardiomyocyte marker” refers to, without limitation, proteins, peptides, nucleic acids, polymorphism of proteins and nucleic acids, splice variants, fragments of proteins or nucleic acids, elements, and other analytes which are specifically expressed or present in endogenous cardiomyocytes. Exemplary cardiomyocyte markers include, but are not limited to, cardiac troponin T (TNNT2), cardiac troponin I (TNNI3), potassium channel KCNJ2, repressor element-1 silencing transcription actor (REST), ryanodine receptor (RyR), sodium channel (SCN5a), and those described in Yang et al. Circ. Res. 2014; 114 (3): 511-23.

The term “immature cardiomyocyte” as used herein is meant a cardiomyocyte that is immature (e.g., electrical, metabolic, and/or contractile). Immature cardiomyocytes display automaticity or pacemaker-like activity, have a higher resting membrane potential and slower upstroke velocity, low expression of skeletal troponin I, have a less organized sarcomere structure, lower maximum contractile force, do not have T-tubules, predominantly acquire energy through glycolysis (rather than oxidative phosphorylation), and may be a senescent state rather than a quiescent state.

As used herein, the term “proliferation” means growth and division of cells. In some embodiments, the term “proliferation” as used herein in reference to cells refers to a group of cells that can increase in number over a period of time.

In the context of cell ontogeny, the adjective “differentiated”, or “differentiating” is a relative term meaning a “differentiated cell” is a cell that has progressed further down the developmental pathway than the cell it is being compared with. Thus, stem cells can differentiate to lineage-restricted precursor cells (such as a mesodermal stem cell), which in turn can differentiate into other types of precursor cells further down the pathway (such as a cardiomyocyte precursors), and then to an end-stage differentiated cell, which plays a characteristic role in a certain tissue type, and may or may not retain the capacity to proliferate further.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaccous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecules having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

As used herein, the term “contacting” (e.g., contacting at least one immature cardiomyocyte or a precursor thereof with a maturation factor, or combination of maturation factors) is intended to include incubating the differentiation medium and/or agent and the cell together in vitro (e.g., adding the maturation factors to cells in culture). In some embodiments, the term “contacting” is not intended to include the in vivo exposure of cells to the compounds as disclosed herein that may occur naturally in a subject (e.g., exposure that may occur as a result of a natural physiological process). In some embodiments, the term “contacting” is intended to include co-culturing at least one immature cardiomyocyte with at least one secondary cell (e.g., at least one endothelial cell). The step of contacting at least one immature cardiomyocyte or a precursor thereof with a maturation factor as in the embodiments described herein can be conducted in any suitable manner. For example, the cells may be treated in three-dimensional culture. In some embodiments, the cells are treated in conditions that promote the formation of cardiomyocytes. The disclosure contemplates any conditions which promote the formation of mature cardiomyocytes. Examples of conditions that promote the formation of mature cardiomyocytes include, without limitation, suspension culture in low attachment tissue culture plates, spinner flasks, and aggrewell plates. In some embodiments, the inventors have observed that mature cardiomyocytes have remained stable in media. In some aspects, serum (e.g., heat inactivated fetal bovine serum) is added prior to dissociating and re-plating the cells.

It is understood that the cells contacted with a maturation factor (e.g., a cardiomyocyte maturation factor) can also be simultaneously or subsequently contacted with another agent, such as other differentiation agents or environments to stabilize the cells, or to differentiate or mature the cells further.

Similarly, at least one immature cardiomyocyte or a precursor thereof can be contacted with at least one cardiomyocyte maturation factor and then contacted with at least another cardiomyocyte maturation factor. In some embodiments, the cell is contacted with at least one cardiomyocyte maturation factor, and the contact is temporally separated, and in some embodiments, a cell is contacted with at least one cardiomyocyte maturation factor substantially simultaneously. In some embodiments, the cell is contacted with at least two, at least three, at least four, at least five, at least six, at least seven, at least eight, at least nine, or at least 10 cardiomyocyte maturation factors

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other. In some embodiments, a cell line comprises a cardiomyocyte described herein.

The term “exogenous” refers to a substance present in a cell or organism other than its native source. For example, the terms “exogenous nucleic acid” or “exogenous protein” refer to a nucleic acid or protein that has been introduced by a process involving the hand of man into a biological system such as a cell or organism in which it is not normally found or in which it is found in lower amounts. A substance will be considered exogenous if it is introduced into a cell or an ancestor of the cell that inherits the substance. In contrast, the term “endogenous” refers to a substance that is native to the biological system.

The terms “genetically modified” or “engineered” cell as used herein refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. The process of transferring the nucleic into the cell can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell. It should be appreciated that the term genetically modified is intended to include the introduction of a modified RNA directly into a cell (e.g., a synthetic, modified RNA). Such synthetic modified RNAs include modifications to prevent rapid degradation by endo- and exo-nucleases and to avoid or reduce the cell's innate immune or interferon response to the RNA. Modifications include, but are not limited to, for example, (a) end modifications, e.g., 5′ end modifications (phosphorylation dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages. To the extent that such modifications interfere with translation (i.e., results in a reduction of 50% or more in translation relative to the lack of the modification—e.g., in a rabbit reticulocyte in vitro translation assay), the modification is not suitable for the methods and compositions described herein.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene and polypeptides obtained by translation of mRNA transcribed from a gene.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a population of cardiomyocytes, refers to a population of cells that contain fewer than about 20%, more preferably fewer than about 15%, 10%, 8%, 7%, most preferably fewer than about 5%, 4%, 3%, 2%, 1%, or less than 1%, of cells that are not cardiomyocytes as defined by the terms herein. In some embodiments, the present invention encompasses methods to expand a population of cardiomyocytes, wherein the expanded population of cardiomyocytes is a substantially pure population of cardiomyocytes.

The terms “enriching” or “enriched” are used interchangeably herein and mean that the yield (fraction) of cells of one type is increased by at least 10% over the fraction of cells of that type in the starting culture or preparation.

The terms “renewal” or “self-renewal” or “proliferation” are used interchangeably herein, are used to refer to the ability of stem cells to renew themselves by dividing into the same non-specialized cell type over long periods, and/or many months to years. In some instances, proliferation refers to the expansion of cells by the repeated division of single cells into two identical daughter cells.

The term “modulate” is used consistently with its use in the art, i.e., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

A “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate or dedifferentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

In some embodiments the selectable marker confers a proliferation and/or survival advantage on cells that express it relative to cells that do not express it or that express it at significantly lower levels. Such proliferation and/or survival advantage typically occurs when the cells are maintained under certain conditions, i.e., “selective conditions.” To ensure an effective selection, a population of cells can be maintained under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection”, and the marker is said to be “useful for positive selection”. Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

The terms “treat”, “treating”, “treatment”, etc., as applied to an isolated cell, include subjecting the cell to any kind of process or condition or performing any kind of manipulation or procedure on the cell. As applied to a subject, the terms “treat”, “treating”, “treatment”, etc. refer to providing medical or surgical attention, care, or management to an individual. The individual is usually ill or injured, or at increased risk of becoming ill relative to an average member of the population and in need of such attention, care, or management. It may include administering to a subject an effective amount of a composition so that the subject exhibits a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. The term “treatment” includes prophylaxis. Those in need of treatment include those already diagnosed with a condition (e.g., muscle disorder or disease), as well as those likely to develop a condition due to genetic susceptibility or other factors.

The term “tissue” refers to a group or layer of specialized cells which together perform certain special functions. The term “tissue-specific” refers to a source of cells from a specific tissue.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90%, or up to and including a 100% decrease (i.e. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90%, or up to and including a 100% increase or any increase between 10-100% as compared to a reference level, or at least about a 2-fold, or at least about a 3-fold, or at least about a 4-fold, or at least about a 5-fold, or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

### Stem Cells

Stem cells are cells that retain the ability to renew themselves through mitotic cell division and can differentiate into a diverse range of specialized cell types. The two broad types of mammalian stem cells are: embryonic stem (ES) cells that are found in blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

While certain embodiments are described below in reference to the use of stem cells for producing cardiomyocytes (e.g., mature cardiomyocytes) or precursors thereof, germ cells may be used in place of, or with, the stem cells to provide at least one cardiomyocyte, using similar protocols as the illustrative protocols described herein. Suitable germ cells can be prepared, for example, from primordial germ cells present in human fetal material taken about 8-11 weeks after the last menstrual period. Illustrative germ cell preparation methods are described, for example, in Shamblott et al., Proc. Natl. Acad. Sci. USA 95:13726, 1998 and U.S. Pat. No. 6,090,622.

ES cells, e.g., human embryonic stem cells (hESCs) or mouse embryonic stem cells (mESCs), with a virtually endless replication capacity and the potential to differentiate into most cell types, present, in principle, an unlimited starting material to generate the differentiated cells for clinical therapy (stemcells.nih.gov/info/scireport/2006report.htm, 2006). One possible application of ES cells is to generate new cardiomyocytes for the cell replacement therapy of heart failure (e.g., chronic heart failure), by first producing cardiac progenitors, from, e.g., hESCs, and then further differentiating the cardiac progenitors into at least one immature cardiomyocyte or precursor thereof, and then further differentiating the at least one immature cardiomyocyte or precursor thereof into a cardiomyocyte (e.g., mature cardiomyocyte).

hESC cells, are described, for example, by Cowan et al. (N Engl. J. Med. 350:1353, 2004) and Thomson et al. (Science 282:1145, 1998); embryonic stem cells from other primates, Rhesus stem cells (Thomson et al., Proc. Natl. Acad. Sci. USA 92:7844, 1995), marmoset stem cells (Thomson et al., Biol. Reprod. 55:254, 1996) and human embryonic germ (hEG) cells (Shamblott et al., Proc. Natl. Acad. Sci. USA 95:13726, 1998) may also be used in the methods disclosed herein. mESCs, are described, for example, by Tremml et al. (Curr Protoc Stem Cell Biol. Chapter 1: Unit 1C.4, 2008). The stem cells may be, for example, unipotent, totipotent, multipotent, or pluripotent. In some examples, any cells of primate origin that are capable of producing progeny that are derivatives of at least one germinal layer, or all three germinal layers, may be used in the methods disclosed herein.

In certain examples, ES cells may be isolated, for example, as described in Cowan et al. (N Engl. J. Med. 350:1353, 2004) and U.S. Pat. No. 5,843,780 and Thomson et al., Proc. Natl. Acad. Sci. USA 92:7844, 1995. For example, hESCs cells can be prepared from human blastocyst cells using the techniques described by Thomson et al. (U.S. Pat. No. 6,200,806; Science 282:1145, 1998; Curr. Top. Dev. Biol. 38:133 ff., 1998) and Roubinoff et al, Nature Biotech. 18:399, 2000. Equivalent cell types to hESCs include their pluripotent derivatives, such as primitive ectoderm-like (EPL) cells, as outlined, for example, in WO 01/51610 (Bresagen). hESCs can also be obtained from human pre-implantation embryos. Alternatively, in vitro fertilized (IVF) embryos can be used, or one-cell human embryos can be expanded to the blastocyst stage (Bongso et al., Hum Reprod 4:706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses can be isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers. After 9 to 15 days, inner cell mass-derived outgrowths can be dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology can be individually selected by micropipette, mechanically dissociated into clumps, and replated. ES-like morphology is characterized as compact colonies with apparently high nucleus to cytoplasm ratio and prominent nucleoli. Resulting hESCs can then be routinely split every 1-2 weeks, for example, by brief trypsinization, exposure to Dulbecco's PBS (containing 2 mM EDTA), exposure to type IV collagenase (about 200 U/mL; Gibco) or by selection of individual colonies by micropipette. In some examples, clump sizes of about 50 to 100 cells are optimal. mESCs cells can be prepared from using the techniques described by e.g., Conner et al. (Curr. Prot. in Mol. Biol. Unit 23.4, 2003).

Embryonic stem cells can be isolated from blastocysts of members of the primate species (U.S. Pat. No. 5,843,780; Thomson et al., Proc. Natl. Acad. Sci. USA 92:7844, 1995). Human embryonic stem (hES) cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hES cells include their pluripotent derivatives, such as primitive ectoderm-like (EPL) cells, as outlined in WO 01/51610 (Bresagen).

Alternatively, in some embodiments, hES cells can be obtained from human preimplantation embryos. Alternatively, in vitro fertilized (IVF) embryos can be used, or one-cell human embryos can be expanded to the blastocyst stage (Bongso et al., Hum Reprod 4:706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses are isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers.

After 9 to 15 days, inner cell mass-derived outgrowths are dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology are individually selected by micropipette, mechanically dissociated into clumps, and replated. ES-like morphology is characterized as compact colonies with apparently high nucleus to cytoplasm ratio and prominent nucleoli. Resulting ES cells are then routinely split every 1-2 weeks by brief trypsinization, exposure to Dulbecco's PBS (containing 2 mM EDTA), exposure to type IV collagenase (^{˜}200 U/mL; Gibco) or by selection of individual colonies by micropipette. Clump sizes of about 50 to 100 cells are optimal.

In some embodiments, human Embryonic Germ (hEG) cells are pluripotent stem cells which can be used in the methods as disclosed herein to differentiate into primitive endoderm cells. hEG cells can be used be prepared from primordial germ cells present in human fetal material taken about 8-11 weeks after the last menstrual period. Suitable preparation methods are described in Shamblott et al., Proc. Natl. Acad. Sci. USA 95:13726, 1998 and U.S. Pat. No. 6,090,622, which is incorporated herein in its entirety by reference.

Briefly, genital ridges processed to form disaggregated cells. EG growth medium is DMEM, 4500 mg/L D-glucose, 2200 mg/L mM NaHCO₃; 15% ES qualified fetal calf serum (BRL); 2 mM glutamine (BRL); 1 mM sodium pyruvate (BRL); 1000-2000 U/mL human recombinant leukemia inhibitory factor (LIF, Genzyme); 1-2 ng/mL human recombinant bFGF (Genzyme); and 10 μM forskolin (in 10% DMSO). Ninety-six well tissue culture plates are prepared with a sub-confluent layer of feeder cells (e.g., STO cells, ATCC No. CRL 1503) cultured for 3 days in modified EG growth medium free of LIF, bFGF or forskolin, inactivated with 5000 rad y-irradiation “0.2 mL of primary germ cell (PGC) suspension is added to each of the wells. The first passage is done after 7-10 days in EG growth medium, transferring each well to one well of a 24-well culture dish previously prepared with irradiated STO mouse fibroblasts. The cells are cultured with daily replacement of medium until cell morphology consistent with EG cells is observed, typically after 7-30 days or 1-4 passages.

In certain examples, the stem cells can be undifferentiated (e.g. a cell not committed to a specific linage) prior to exposure to at least one cardiomyocyte maturation factor according to the methods as disclosed herein, whereas in other examples it may be desirable to differentiate the stem cells to one or more intermediate cell types prior to exposure of the at least one cardiomyocyte maturation factor(s) described herein. For example, the stem cells may display morphological, biological or physical characteristics of undifferentiated cells that can be used to distinguish them from differentiated cells of embryo or adult origin. In some examples, undifferentiated cells may appear in the two dimensions of a microscopic view in colonies of cells with high nuclear/cytoplasmic ratios and prominent nucleoli. The stem cells may be themselves (for example, without substantially any undifferentiated cells being present) or may be used in the presence of differentiated cells. In certain examples, the stem cells may be cultured in the presence of suitable nutrients and optionally other cells such that the stem cells can grow and optionally differentiate. For example, embryonic fibroblasts or fibroblast-like cells may be present in the culture to assist in the growth of the stem cells. The fibroblast may be present during one stage of stem cell growth but not necessarily at all stages. For example, the fibroblast may be added to stem cell cultures in a first culturing stage and not added to the stem cell cultures in one or more subsequent culturing stages.

Stem cells used in all aspects of the present invention can be any cells derived from any kind of tissue (for example embryonic tissue such as fetal or pre-fetal tissue, or adult tissue), which stem cells have the characteristic of being capable under appropriate conditions of producing progeny of different cell types, e.g. derivatives of all of at least one of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). In some embodiments, the source of human stem cells or pluripotent stem cells used for chemically-induced differentiation into mature cardiomyocytes did not involve destroying a human embryo.

In another embodiment, the stem cells can be isolated from tissue including solid tissue. In some embodiments, the tissue is skin, fat tissue (e.g. adipose tissue), muscle tissue, heart or cardiac tissue. In other embodiments, the tissue is for example but not limited to, umbilical cord blood, placenta, bone marrow, or chondral.

Stem cells of interest also include embryonic cells of various types, exemplified by human embryonic stem (hES) cells, described by Thomson et al. (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78 (2): 205-16; etc.) The stem cells may be obtained from any mammalian species, e.g. human, equine, bovine, porcine, canine, feline, rodent, e.g. mice, rats, hamster, primate, etc. In some embodiments, a human embryo was not destroyed for the source of pluripotent cell used on the methods and compositions as disclosed herein.

ES cells are considered to be undifferentiated when they have not committed to a specific differentiation lineage. Such cells display morphological characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection. For example, see U.S. application Ser. No. 2003/0224411 A1; Bhattacharya (2004) Blood 103 (8): 2956-64; and Thomson (1998), supra., each herein incorporated by reference. Human ES cell lines express cell surface markers that characterize undifferentiated nonhuman primate ES and human EC cells, including stage-specific embryonic antigen (SSEA)-3, SSEA-4, TRA-1-60, TRA-1-81, and alkaline phosphatase. The globo-series glycolipid GL7, which carries the SSEA-4 epitope, is formed by the addition of sialic acid to the globo-series glycolipid GbS, which carries the SSEA-3 epitope. Thus, GL7 reacts with antibodies to both SSEA-3 and SSEA-4. The undifferentiated human ES cell lines did not stain for SSEA-1, but differentiated cells stained strongly for SSEA-I. Methods for proliferating hES cells in the undifferentiated form are described in WO 99/20741, WO 01/51616, and WO 03/020920.

A mixture of cells from a suitable source of endothelial, muscle, and/or neural stem cells can be harvested from a mammalian donor by methods known in the art. A suitable source is the hematopoietic microenvironment. For example, circulating peripheral blood, preferably mobilized (i.e., recruited), may be removed from a subject. Alternatively, bone marrow may be obtained from a mammal, such as a human patient, undergoing an autologous transplant. In some embodiments, stem cells can be obtained from the subjects adipose tissue, for example using the CELUTION™ SYSTEM from Cytori, as disclosed in U.S. Pat. Nos. 7,390,484 and 7,429,488 which is incorporated herein in its entirety by reference.

In some embodiments, human umbilical cord blood cells (HUCBC) are useful in the methods as disclosed herein. Human UBC cells are recognized as a rich source of hematopoietic and mesenchymal progenitor cells (Broxmeyer et al., 1992 Proc. Natl. Acad. Sci. USA 89:4109-4113). Previously, umbilical cord and placental blood were considered a waste product normally discarded at the birth of an infant. Cord blood cells are used as a source of transplantable stem and progenitor cells and as a source of marrow repopulating cells for the treatment of malignant diseases (i.e. acute lymphoid leukemia, acute myeloid leukemia, chronic myeloid leukemia, myelodysplastic syndrome, and nueroblastoma) and non-malignant diseases such as Fanconi's anemia and aplastic anemia (Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503). A distinct advantage of HUCBC is the immature immunity of these cells that is very similar to fetal cells, which significantly reduces the risk for rejection by the host (Taylor & Bryson, 1985 J. Immunol. 134:1493-1497). Human umbilical cord blood contains mesenchymal and hematopoietic progenitor cells, and endothelial cell precursors that can be expanded in tissue culture (Broxmeyer et al., 1992 Proc. Natl. Acad. Sci. USA 89:4109-4113; Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503; Taylor & Bryson, 1985 J. Immunol. 134:1493-1497; Broxmeyer, 1995 Transfusion 35:694-702; Chen et al., 2001 Stroke 32:2682-2688; Nicda et al., 1997 Br. J. Haematology 98:775-777; Erices et al., 2000 Br. J. Haematology 109:235-242). The total content of hematopoietic progenitor cells in umbilical cord blood equals or exceeds bone marrow, and in addition, the highly proliferative hematopoietic cells are eightfold higher in HUCBC than in bone marrow and express hematopoietic markers such as CD14, CD34, and CD45 (Sanchez-Ramos et al., 2001 Exp. Neur. 171:109-115; Bicknese et al., 2002 Cell Transplantation 11:261-264; Lu et al., 1993 J. Exp Med. 178:2089-2096).

In another embodiment, pluripotent cells are cells in the hematopoietic micro-environment, such as the circulating peripheral blood, preferably from the mononuclear fraction of peripheral blood, umbilical cord blood, bone marrow, fetal liver, or yolk sac of a mammal. The stem cells, especially neural stem cells, may also be derived from the central nervous system, including the meninges.

In another embodiment, pluripotent cells are present in embryoid bodies are formed by harvesting ES cells with brief protease digestion, and allowing small clumps of undifferentiated human ESCs to grow in suspension culture. Differentiation is induced by withdrawal of conditioned medium. The resulting embryoid bodies are plated onto semi-solid substrates. Formation of differentiated cells may be observed after around about 7 days to around about 4 weeks. Viable differentiating cells from in vitro cultures of stem cells are selected for by partially dissociating embryoid bodies or similar structures to provide cell aggregates. Aggregates comprising cells of interest are selected for phenotypic features using methods that substantially maintain the cell to cell contacts in the aggregate.

In an alternative embodiment, the stem cells can be reprogrammed stem cells, such as stem cells derived from somatic or differentiated cells. In such an embodiment, the de-differentiated stem cells can be for example, but not limited to, neoplastic cells, tumor cells and cancer cells or alternatively induced reprogrammed cells such as induced pluripotent stem cells or iPS cells. Reprogrammed iPS cells may be obtained using any method known to those of skill in the art. For example, reprogrammed iPS cells may be obtained using one or more transcription factors. In one embodiment, iPSC cells are obtained via reprogramming, e.g., reprogramming somatic cells, using one or more transcription factors including, but not limited to, Oct4, Sox2, Klf4, and c-Myc. Additional methods for making reprogrammed iPS cells are described in WO 2013/177133 and WO 2022/204567, both of which are incorporated herein by reference.

### Cloning and Cell Culture

Illustrative methods for molecular genetics and genetic engineering that may be used in the technology described herein may be found, for example, in current editions of Molecular Cloning: A Laboratory Manual, (Sambrook et al., Cold Spring Harbor); Gene Transfer Vectors for Mammalian Cells (Miller & Calos eds.); and Current Protocols in Molecular Biology (F. M. Ausubel et al. eds., Wiley & Sons). Cell biology, protein chemistry, and antibody techniques can be found, for example, in Current Protocols in Protein Science (J. E. Colligan et al. eds., Wiley & Sons); Current Protocols in Cell Biology (J. S. Bonifacino et al., Wiley & Sons) and Current protocols in Immunology (J. E. Colligan et al. eds., Wiley & Sons.). Illustrative reagents, cloning vectors, and kits for genetic manipulation may be commercially obtained, for example, from BioRad, Stratagene, Invitrogen, ClonTech, and Sigma-Aldrich Co.

Suitable cell culture methods may be found, for example, in the current edition of Culture of Animal Cells: A Manual of Basic Technique (R. I. Freshney ed., Wiley & Sons); General Techniques of Cell Culture (M. A. Harrison & I. F. Rac, Cambridge Univ. Press), and Embryonic Stem Cells: Methods and Protocols (K. Turksen ed., Humana Press). Suitable tissue culture supplies and reagents are commercially available, for example, from Gibco/BRL, Nalgene-Nunc International, Sigma Chemical Co., and ICN Biomedicals.

Pluripotent stem cells can be propagated by one of ordinary skill in the art and continuously in culture, using culture conditions that promote proliferation without promoting differentiation. Exemplary serum-containing ES medium is made with 80% DMEM (such as Knock-Out DMEM, Gibco), 20% of either defined fetal bovine serum (FBS, Hyclone) or serum replacement (WO 98/30679), 1% non-essential amino acids, 1 mM L-glutamine, and 0.1 mM β-mercaptoethanol. Just before use, human bFGF is added to 4 ng/ml (WO 99/20741, Geron Corp.). Traditionally, ES cells are cultured on a layer of feeder cells, typically fibroblasts derived from embryonic or fetal tissue.

Alternatively, pluripotent SCs can be maintained in an undifferentiated state even without feeder cells. The environment for feeder-free cultures includes a suitable culture substrate, particularly an extracellular matrix such as MATRIGEL® (gelatinous protein mixture) or laminin. Typically, enzymatic digestion is halted before cells become completely dispersed (˜5 min with collagenase IV). Clumps of ˜10 to 2,000 cells are then plated directly onto the substrate without further dispersal.

### Generating Cardiomyocytes

Aspects of the disclosure relate to generating cardiomyocytes (e.g., mature cardiomyocytes). Generally, the cardiomyocytes produced according to the methods disclosed herein demonstrate several hallmarks of functional mature cardiomyocytes, including, but not limited to, being electrically mature (e.g., exhibit decreased automaticity), contractility mature, and metabolically mature.

The cardiomyocytes can be produced according to any suitable culturing protocol or series of culturing protocols to differentiate a stem cell or pluripotent cell to a desired stage of differentiation. In some embodiments, the cardiomyocytes or the precursors thereof are produced by culturing at least one pluripotent cell for a period of time and under conditions suitable for the at least one pluripotent cell to differentiate into the cardiomyocytes or the precursors thereof. In some embodiments, the cardiomyocytes are produced by shifting an immature cardiomyocyte from a senescent state to a quiescent state, thereby enhancing maturation of the cardiomyocytes.

In some embodiments, the cardiomyocytes are a substantially pure population of cardiomyocytes. In some embodiments, a population of cardiomyocytes or precursors thereof comprises a mixture of pluripotent cells or differentiated cells. In some embodiments, a population of cardiomyocytes or precursors thereof is substantially free or devoid of embryonic stem cells or pluripotent cells or iPS cells.

In some embodiments, a somatic cell, e.g., a fibroblast, can be isolated from a subject, for example as a tissue biopsy, such as, for example, a skin biopsy, and reprogrammed into an induced pluripotent stem cell for further differentiation to produce a cardiomyocyte or precursor thereof for use in the compositions and methods described herein. In some embodiments, a somatic cell, e.g., a fibroblast, is maintained in culture by methods known by one of ordinary skill in the art, and in some embodiments, propagated prior to being converted into cardiomyocytes by the methods as disclosed herein.

In some embodiments, the cardiomyocytes or precursors thereof are maintained in culture by methods known by one of ordinary skill in the art, and in some embodiments, propagated prior to being converted into cardiomyocytes by the methods as disclosed herein.

Further, cardiomyocytes or precursors thereof, e.g., immature cardiomyocytes, can be from any mammalian species, with non-limiting examples including a murine, bovine, simian, porcine, equine, ovine, or human cell. For clarity and simplicity, the description of the methods herein refers to a mammalian cardiomyocytes or precursor thereof, but it should be understood that all of the methods described herein can be readily applied to other cell types of cardiomyocytes or precursors thereof. In some embodiments, the cardiomyocytes or precursors thereof are derived from a human individual.

Aspects of the disclosure involve immature cardiomyocytes. Immature cardiomyocytes of use herein can be derived from any source or generated in accordance with any suitable protocol. In some aspects, pluripotent stem cells, e.g., iPSCs or hESCs, are differentiated to immature cardiomyocytes. In some aspects, the immature cardiomyocytes are further matured to mature cardiomyocytes. In some embodiments, pluripotent stem cells are differentiated to immature cardiomyocytes using a differentiation protocol described by Lian et al. (Nat Protoc. 2012; 8 (1): 162-175), which is incorporated herein by reference. In some embodiments, the differentiation protocol described by Lian was modified as described herein. In some embodiments, pluripotent stem cells are contacted with one or more small molecules to manipulate the Wnt pathway, and thereby differentiate the pluripotent stem cells into immature cardiomyocytes. In some aspects, the one or more small molecules are selected from the group consisting of CHIR 99021 and IWP4. In some embodiments, a population of pluripotent stem cells is contacted with a first Wnt pathway modulator (e.g., CHIR 99021), and is then contacted with a second Wnt pathway modulator (e.g., IWP4). Additional methods for producing cardiomyocytes are described by U.S. Pat. No. 9,452,201; WO 2014/200339; and WO 2017/039445, which are incorporated herein by reference.

Aspects of the disclosure involve cardiomyocytes (e.g., mature cardiomyocytes). Cardiomyocytes of use herein can be derived from any source or generated in accordance with any suitable protocol. In some aspects, immature cardiomyocytes are induced to mature into mature cardiomyocytes. In some aspects, senescent cardiomyocytes (e.g., immature cardiomyocytes) are induced into quiescent cardiomyocytes (e.g., mature cardiomyocytes). Cellular quiescence may facilitate cardiomyocyte maturation.

In some aspects, the disclosure provides a method for generating mature cardiomyocytes (e.g., electrically mature, contractility mature, and/or metabolically mature) from immature cardiomyocytes, the method comprising co-culturing immature cardiomyocytes with endothelial cells. In some aspects, immature cardiomyocytes are cultured with conditioned media from endothelial cells. In some embodiments, the immature cardiomyocytes are derived from stem cells (e.g., iPSCs or hESCs). In some embodiments, the immature cardiomyocytes are iPSC-derived cardiomyocytes. In some embodiments, the immature cardiomyocytes are human iPSC-derived cardiomyocytes. In some embodiments, the endothelial cells are derived from stem cells (e.g., iPSCs or hESCs). In some embodiments, the endothelial cells are iPSC-derived endothelial cells. In some embodiments, the endothelial cells are human iPSC-derived endothelial cells.

In some embodiments, the immature cardiomyocytes and endothelial cells are co-cultured for a period of at least 4, 6, 8, 10, 12, 14, 16, 18, 20, 22, 24, 26, 28, 30, 32, 34, 36, or 38 days. In some embodiments, the immature cardiomyocytes and endothelial cells are co-cultured for a period of less than 40, 38, 36, 34, 32, 30, 28, 26, 24, 22, 20, 18, 16, 14, 12, 10, 8, or 6 days. In some embodiments, the immature cardiomyocytes and endothelial cells are co-cultured for a period of 5 to 35 days, 5 to 30 days, 5 to 25 days, 5 to 20 days, 5 to 15 days, 5 to 10 days, 10 to 35 days, 10 to 30 days, 10 to 25 days, 10 to 20 days, 10 to 15 days, 15 to 35 days, 15 to 30 days, 15 to 25 days, 15 to 20 days, 20 to 35 days, 20 to 30 days, 20 to 25 days, 25 to 35 days, or 25 to 30 days. In some embodiments, the immature cardiomyocytes and endothelial cells are cultured at a ratio between 5:1 to 1:5 cardiomyocytes to endothelial cells, e.g., at the time of plating. In some embodiments, the immature cardiomyocytes and endothelial cells are cultured at a ratio of 10:1, 9:1, 8:1, 8:1, 7:1, 6:1, 5:1, 4:1, 3:1, 2:1, 1:1, 1:2, 1:3, 1:4, 1:5, 1:6, 1:7, 1:8, 1:9, or 1:10 cardiomyocyte to endothelial cell, e.g., at the time of plating.

Aspects of the disclosure involve generating cardiomyocytes which resemble endogenous mature cardiomyocytes in form and function, but nevertheless are distinct from native cardiomyocytes. In some embodiments, the cardiomyocytes generated from the co-culture of immature cardiomyocytes with endothelial cells exhibit an increased percentage of live cells. In some embodiments, the cardiomyocytes generated from the co-culture of immature cardiomyocytes with endothelial cells exhibit 0.1 to 100 fold, 1 to 100 fold, 5 to 100 fold, 10 to 100 fold, 25 to 100 fold, 0.1 to 75 fold, 1 to 75 fold, 5 to 75 fold, 10 to 75 fold, 25 to 75 fold, 0.1 to 50 fold, 1 to 50 fold, 5 to 50 fold, 10 to 50 fold, 25 to 50 fold, 0.1 to 25 fold, 1 to 25 fold, 5 to 25 fold, 10 to 25 fold, 0.1 to 10 fold, 1 to 10 fold, 5 to 10 fold, 0.1 to 5 fold, 1 to 5 fold, or 0.1 to 1 fold increased percentage of live cells. In some embodiments, the cardiomyocytes generated from the co-culture of immature cardiomyocytes with endothelial cells exhibit at least a 0.1-fold, 0.2-fold, 0.3-fold, 0.4-fold, 0.5-fold, 0.6-fold, 0.7-fold, 0.8-fold, 0.9-fold, 1-fold, 1.5-fold, 2-fold, 2.5-fold, 3-fold, 3.5-fold, 4-fold, 4.5-fold, 5-fold, 10-fold, 25-fold, 50-fold, or in some aspects greater than a 50-fold increased percentage of live cells.

In some embodiments, the cardiomyocytes generated from the co-culture of immature cardiomyocytes with endothelial cells exhibit increased expression of at least one marker comprising TNNT2, TNNI3, Cx43, CD36, and Kir2.1 (i.e., as compared to immature cardiomyocyte). In some embodiments, the cardiomyocytes generated from the co-culture of immature cardiomyocytes with endothelial cells exhibit 0.1 to 100 fold, 1 to 100 fold, 5 to 100 fold, 10 to 100 fold, 25 to 100 fold, 0.1 to 75 fold, 1 to 75 fold, 5 to 75 fold, 10 to 75 fold, 25 to 75 fold, 0.1 to 50 fold, 1 to 50 fold, 5 to 50 fold, 10 to 50 fold, 25 to 50 fold, 0.1 to 25 fold, 1 to 25 fold, 5 to 25 fold, 10 to 25 fold, 0.1 to 10 fold, 1 to 10 fold, 5 to 10 fold, 0.1 to 5 fold, 1 to 5 fold, or 0.1 to 1 fold increased expression of at least one marker comprising TNNT2, TNNI3, Cx43, CD36, and Kir2.1. In some embodiments, the cardiomyocytes generated from the co-culture of immature cardiomyocytes with endothelial cells exhibit at least a 0.1-fold, 0.2-fold, 0.3-fold, 0.4-fold, 0.5-fold, 0.6-fold, 0.7-fold, 0.8-fold, 0.9-fold, 1-fold, 1.5-fold, 2-fold, 2.5-fold, 3-fold, 3.5-fold, 4-fold, 4.5-fold, 5-fold, 10-fold, 25-fold, 50-fold, or in some aspects greater than a 50-fold increased expression of at least one marker comprising TNNT2, TNNI3, Cx43, CD36, and Kir2.1.

In some embodiments, the cardiomyocytes generated from the co-culture of immature cardiomyocytes with endothelial cells exhibit accelerated maturation (e.g., electrical maturation). In some embodiments, the cardiomyocytes generated from the co-culture of immature cardiomyocytes with endothelial cells exhibit 0.1 to 100 fold, 1 to 100 fold, 5 to 100 fold, 10 to 100 fold, 25 to 100 fold, 0.1 to 75 fold, 1 to 75 fold, 5 to 75 fold, 10 to 75 fold, 25 to 75 fold, 0.1 to 50 fold, 1 to 50 fold, 5 to 50 fold, 10 to 50 fold, 25 to 50 fold, 0.1 to 25 fold, 1 to 25 fold, 5 to 25 fold, 10 to 25 fold, 0.1 to 10 fold, 1 to 10 fold, 5 to 10 fold, 0.1 to 5 fold, 1 to 5 fold, or 0.1 to 1 fold accelerated maturation. In some embodiments, the cardiomyocytes generated from the co-culture of immature cardiomyocytes with endothelial cells exhibit at least a 0.1-fold, 0.2-fold, 0.3-fold, 0.4-fold, 0.5-fold, 0.6-fold, 0.7-fold, 0.8-fold, 0.9-fold, 1-fold, 1.5-fold, 2-fold, 2.5-fold, 3-fold, 3.5-fold, 4-fold, 4.5-fold, 5-fold, 10-fold, 25-fold, 50-fold, or in some aspects greater than a 50-fold accelerated maturation.

In some embodiments, the cardiomyocytes generated from the co-culture of immature cardiomyocytes with endothelial cells exhibit decreased automaticity, a decreased rate of spontaneous beating (e.g., less than 3 beats per minutes), and/or a decreased risk of arrhythmias (i.e., as compared to an immature cardiomyocyte). In some embodiments, the cardiomyocytes generated from the co-culture of immature cardiomyocytes with endothelial cells exhibit 1 to 99%, 5 to 99%, 10 to 99%, 25 to 99%, 50 to 99%, 60 to 99%, 75 to 99%, 1 to 75%, 5 to 75%, 10 to 75%, 15 to 75%, 20 to 75%, 25 to 75%, 30 to 75%, 35 to 75%, 40 to 75%, 45 to 75%, 50 to 75%, 55 to 75%, 60 to 75%, 1 to 50%, 5 to 50%, 10 to 50%, 15 to 50%, 20 to 50%, 25 to 50%, 30 to 50%, 35 to 50%, 40 to 50%, 1 to 25%, 5 to 25%, 10 to 25%, 15 to 25%, 20 to 25%, 1 to 15%, 5 to 15%, 10 to 15%, 5 to 10%, or 1 to 5% decreased automaticity, decreased rate of spontaneous beating (e.g., less than 3 beats per minutes), and/or decreased risk of arrhythmias (i.e., as compared to an immature cardiomyocyte). In some embodiments, the cardiomyocytes generated from the co-culture of immature cardiomyocytes with endothelial cells exhibit at least a 0.1%, 0.5%, 1%, 5%, 10%, 15%, 20%, 25%, 30%, 35%, 40%, 45%, 50%, 55%, 60%, 65%, 70%, 75%, 80%, 85%, 90%, or in some aspects greater than a 90% decrease in automaticity, rate of spontaneous beating (e.g., less than 3 beats per minutes), and/or risk of arrhythmias (i.e., as compared to an immature cardiomyocyte).

In some aspects, the disclosure provides a method for generating mature cardiomyocytes (e.g., electrically mature, contractility mature, and/or metabolically mature) from immature cardiomyocytes, the method comprising contacting a population of cells comprising immature cardiomyocytes with at least one cardiomyocyte maturation factor comprising a regulator of FOXO-FOXM1 signaling (e.g., a FOXO activator and/or a FOXM1 inhibitor), to induce the maturation (e.g., in vitro maturation) of at least one immature cardiomyocyte in the population into a cardiomyocyte. In some embodiments, a population of cells comprising immature cardiomyocytes is contacted with at least one cardiomyocyte maturation factor (e.g., FOXO activator and/or FOXM1 inhibitor). A population of cells comprising immature cardiomyocytes may be contacted with 0.1 to 5 μM, 0.5 to 5 μM, 1 to 5 μM, 1.5 to 5 μM, 2 to 5 μM, 3 to 5 μM, 0.1 to 3 μM, 0.5 to 3 μM, 1 to 3 μM, 0.1 to 1 μM, or 0.5 to 1 μM of at least one cardiomyocyte maturation factor. In some aspects, FOXO is upregulated in combination with FOXM1 inhibition to enhance the maturation of cardiomyocytes derived from stem cells. In some aspects, FOXO is activated without inhibiting FOXM1 to enhance the maturation of cardiomyocytes derived from stem cells. In some aspects, FOXM1 is inhibited without activating FOXO to enhance the maturation of cardiomyocytes derived from stem cells. In some aspects, the population of cells comprises immature cardiomyocytes and endothelial cells.

The disclosure contemplates the use of any FOXO activator that encourages immature cardiomyocytes to differentiate and/or mature into cardiomyocytes (e.g., alone or in combination with another cardiomyocyte maturation factor (e.g., a FOXM1 inhibitor, an mTOR inhibitor, a p53 activator)). Examples of FOXO activators include small molecule, nucleic acid, amino acid, metabolite, polypeptide, antibody and antibody-like molecules, aptamers, macrocycles, and other molecules. In some aspects, FOXO is activated by starvation (e.g., low glucose and/or low insulin culture conditions). In some aspects, a FOXO activator comprises an mTOR inhibitor (e.g., Torin1, Torin2, rapamycin, everolimus, and/or temsirolimus). In some embodiments, a FOXO activator comprises metformin, resveratrol, Selinexor, celecoxib, doxorubicin, carbenoxolone and/or functional and/or structural analogs or variants thereof. In some embodiments, a FOXO activator is LOM612. In one embodiment, a FOXO activator comprises 0.5 to 7 μM, 0.75 to 6 μM, or 1 to 5 μM LOM612. Additional examples of FOXO activators are described in Calissi et al., Nat Rev Drug Discov., 20 (1): 21-38 (2021), which is incorporated herein by reference.

The disclosure contemplates the use of any FOXM1 inhibitor that encourages immature cardiomyocytes to differentiate and/or mature into cardiomyocytes (e.g., alone or in combination with another cardiomyocyte maturation factor (e.g., a FOXO activator, an mTOR inhibitor, a p53 activator)). Examples of FOXM1 inhibitors include small molecule, nucleic acid, amino acid, metabolite, polypeptide, antibody and antibody-like molecules, aptamers, macrocycles, and other molecules. In some aspects, a FOXM1 inhibitor comprises FDI-6, thiostrepton, honokiol, siomycin A, curcumin, bortezomib, MG115, MG132, and/or functional and/or structural analogs or variants thereof. In some embodiments, a FOXM1 inhibitor is RCM1. In one embodiment, a FOXM1 inhibitor comprises 0.1 to 7 μM, 0.15 to 6 μM, or 0.2 to 5 μM RCM1. Additional examples of FOXM1 inhibits are described in Gartel, “Expert Opin Investig Drugs, 19 (2): 235-242 (2010); Chesnokov et al., Cell Death & Disease, 12:704 (2021; and Bhat et al., PLOS ONE, 4 (8): e6593 (2009), each of which is incorporated herein by reference.

In some embodiments, the cardiomyocytes generated from the contacting of immature cardiomyocytes with at least one maturation factor comprising a FOXO activator and/or a FOXM1 inhibitor exhibit enhanced contractility. In some aspects, contractility is assessed by measuring beat amplitude using a multielectrode array. In some embodiments, the cardiomyocytes generated from the contacting of immature cardiomyocytes with at least one maturation factor comprising a FOXO activator and/or a FOXM1 inhibitor exhibit 0.1 to 25 fold, 0.25 to 25 fold, 0.5 to 25 fold, 0.75 to 25 fold, 1 to 25 fold, 1.25 to 25 fold, 1.5 to 25 fold, 1.75 to 25 fold, 2 to 25 fold, 5 to 25 fold, 10 to 25 fold, 15 to 25 fold, 0.1 to 10 fold, 0.25 to 10 fold, 0.5 to 10 fold, 0.75 to 10 fold, 1 to 10 fold, 1.25 to 10 fold, 1.5 to 10 fold, 1.75 to 10 fold, 2 to 10 fold, 5 to 10 fold, 0.1 to 5 fold, 0.25 to 5 fold, 0.5 to 5 fold, 0.75 to 5 fold, 1 to 5 fold, 1.25 to 5 fold, 1.5 to 5 fold, 1.75 to 5 fold, 2 to 5 fold, 0.1 to 3 fold, 0.25 to 3 fold, 0.5 to 3 fold, 0.75 to 3 fold, 1 to 3 fold, 1.25 to 3 fold, 1.5 to 3 fold, 1.75 to 3 fold, 2 to 3 fold, 0.1 to 2 fold, 0.25 to 2 fold, 0.5 to 2 fold, 0.75 to 2 fold, 1 to 2 fold, 1.1 to 2 fold, 1.2 to 2 fold, 1.3 to 2 fold, 1.4 to 2 fold, 1.5 to 2 fold, 1.6 to 2 fold, 1.7 to 2 fold, or 1.8 to 2 fold increase in contractility. In some embodiments, the cardiomyocytes generated from the contacting of immature cardiomyocytes with at least one maturation factor comprising a FOXO activator and/or a FOXM1 inhibitor exhibit at least a 0.1-fold, 0.2-fold, 0.3-fold, 0.4-fold, 0.5-fold, 0.6-fold, 0.7-fold, 0.8-fold, 0.9-fold, 1-fold, 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 3-fold, 3.5-fold, 4-fold, 4.5-fold, or in some aspects greater than a 5-fold, increase in contractility.

In some embodiments, the cardiomyocytes generated from the contacting of immature cardiomyocytes with at least one maturation factor comprising a FOXO activator and/or a FOXM1 inhibitor exhibit increased expression of one or more of TNNT2, TNNI3 and Kir2.1 (i.e., as compared to immature cardiomyocytes). In some embodiments, the cardiomyocytes generated from the contacting of immature cardiomyocytes with at least one maturation factor comprising a FOXO activator and/or a FOXM1 inhibitor exhibit 0.1 to 25 fold, 0.25 to 25 fold, 0.5 to 25 fold, 0.75 to 25 fold, 1 to 25 fold, 1.25 to 25 fold, 1.5 to 25 fold, 1.75 to 25 fold, 2 to 25 fold, 5 to 25 fold, 10 to 25 fold, 15 to 25 fold, 0.1 to 10 fold, 0.25 to 10 fold, 0.5 to 10 fold, 0.75 to 10 fold, 1 to 10 fold, 1.25 to 10 fold, 1.5 to 10 fold, 1.75 to 10 fold, 2 to 10 fold, 5 to 10 fold, 0.1 to 5 fold, 0.25 to 5 fold, 0.5 to 5 fold, 0.75 to 5 fold, 1 to 5 fold, 1.25 to 5 fold, 1.5 to 5 fold, 1.75 to 5 fold, 2 to 5 fold, 0.1 to 3 fold, 0.25 to 3 fold, 0.5 to 3 fold, 0.75 to 3 fold, 1 to 3 fold, 1.25 to 3 fold, 1.5 to 3 fold, 1.75 to 3 fold, 2 to 3 fold, 0.1 to 2 fold, 0.25 to 2 fold, 0.5 to 2 fold, 0.75 to 2 fold, 1 to 2 fold, 1.1 to 2 fold, 1.2 to 2 fold, 1.3 to 2 fold, 1.4 to 2 fold, 1.5 to 2 fold, 1.6 to 2 fold, 1.7 to 2 fold, or 1.8 to 2 fold increase in expression of one or more of TNNT2, TNNI3 and Kir2.1. In some embodiments, the cardiomyocytes generated from the contacting of immature cardiomyocytes with at least one maturation factor comprising a FOXO activator and/or a FOXM1 inhibitor exhibit at least a 0.1-fold, 0.2-fold, 0.3-fold, 0.4-fold, 0.5-fold, 0.6-fold, 0.7-fold, 0.8-fold, 0.9-fold, 1-fold, 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 3-fold, 3.5-fold, 4-fold, 4.5-fold, or in some aspects greater than a 5-fold, increase in expression of one or more of TNNT2, TNNI3 and Kir2.1.

In some embodiments, the cardiomyocytes generated from the contacting of immature cardiomyocytes with at least one maturation factor comprising a FOXO activator and/or a FOXM1 inhibitor exhibit increased mean beat amplitude and/or upstroke velocity (i.e., as compared to immature cardiomyocytes). In some embodiments, the cardiomyocytes generated from the contacting of immature cardiomyocytes with at least one maturation factor comprising a FOXO activator and/or a FOXM1 inhibitor exhibit 0.1 to 25 fold, 0.25 to 25 fold, 0.5 to 25 fold, 0.75 to 25 fold, 1 to 25 fold, 1.25 to 25 fold, 1.5 to 25 fold, 1.75 to 25 fold, 2 to 25 fold, 5 to 25 fold, 10 to 25 fold, 15 to 25 fold, 0.1 to 10 fold, 0.25 to 10 fold, 0.5 to 10 fold, 0.75 to 10 fold, 1 to 10 fold, 1.25 to 10 fold, 1.5 to 10 fold, 1.75 to 10 fold, 2 to 10 fold, 5 to 10 fold, 0.1 to 5 fold, 0.25 to 5 fold, 0.5 to 5 fold, 0.75 to 5 fold, 1 to 5 fold, 1.25 to 5 fold, 1.5 to 5 fold, 1.75 to 5 fold, 2 to 5 fold, 0.1 to 3 fold, 0.25 to 3 fold, 0.5 to 3 fold, 0.75 to 3 fold, 1 to 3 fold, 1.25 to 3 fold, 1.5 to 3 fold, 1.75 to 3 fold, 2 to 3 fold, 0.1 to 2 fold, 0.25 to 2 fold, 0.5 to 2 fold, 0.75 to 2 fold, 1 to 2 fold, 1.1 to 2 fold, 1.2 to 2 fold, 1.3 to 2 fold, 1.4 to 2 fold, 1.5 to 2 fold, 1.6 to 2 fold, 1.7 to 2 fold, or 1.8 to 2 fold increase in mean beat amplitude and/or upstroke velocity. In some embodiments, the cardiomyocytes generated from the contacting of immature cardiomyocytes with at least one maturation factor comprising a FOXO activator and/or a FOXM1 inhibitor exhibit at least a 0.1-fold, 0.2-fold, 0.3-fold, 0.4-fold, 0.5-fold, 0.6-fold, 0.7-fold, 0.8-fold, 0.9-fold, 1-fold, 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 3-fold, 3.5-fold, 4-fold, 4.5-fold, or in some aspects greater than a 5-fold, increase in mean beat amplitude and/or upstroke velocity.

In some embodiments, the cardiomyocytes generated from the contacting of immature cardiomyocytes with at least one maturation factor comprising a FOXO activator and/or a FOXM1 inhibitor exhibit increased maximum oxygen consumption rate (OCR) and/or respiratory reserve capacity (i.e., as compared to immature cardiomyocytes). In some embodiments, the cardiomyocytes generated from the contacting of immature cardiomyocytes with at least one maturation factor comprising a FOXO activator and/or a FOXM1 inhibitor exhibit 0.1 to 25 fold, 0.25 to 25 fold, 0.5 to 25 fold, 0.75 to 25 fold, 1 to 25 fold, 1.25 to 25 fold, 1.5 to 25 fold, 1.75 to 25 fold, 2 to 25 fold, 5 to 25 fold, 10 to 25 fold, 15 to 25 fold, 0.1 to 10 fold, 0.25 to 10 fold, 0.5 to 10 fold, 0.75 to 10 fold, 1 to 10 fold, 1.25 to 10 fold, 1.5 to 10 fold, 1.75 to 10 fold, 2 to 10 fold, 5 to 10 fold, 0.1 to 5 fold, 0.25 to 5 fold, 0.5 to 5 fold, 0.75 to 5 fold, 1 to 5 fold, 1.25 to 5 fold, 1.5 to 5 fold, 1.75 to 5 fold, 2 to 5 fold, 0.1 to 3 fold, 0.25 to 3 fold, 0.5 to 3 fold, 0.75 to 3 fold, 1 to 3 fold, 1.25 to 3 fold, 1.5 to 3 fold, 1.75 to 3 fold, 2 to 3 fold, 0.1 to 2 fold, 0.25 to 2 fold, 0.5 to 2 fold, 0.75 to 2 fold, 1 to 2 fold, 1.1 to 2 fold, 1.2 to 2 fold, 1.3 to 2 fold, 1.4 to 2 fold, 1.5 to 2 fold, 1.6 to 2 fold, 1.7 to 2 fold, or 1.8 to 2 fold increase in maximum oxygen consumption rate and/or respiratory reserve capacity. In some embodiments, the cardiomyocytes generated from the contacting of immature cardiomyocytes with at least one maturation factor comprising a FOXO activator and/or a FOXM1 inhibitor exhibit at least a 0.1-fold, 0.2-fold, 0.3-fold, 0.4-fold, 0.5-fold, 0.6-fold, 0.7-fold, 0.8-fold, 0.9-fold, 1-fold, 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 3-fold, 3.5-fold, 4-fold, 4.5-fold, or in some aspects greater than a 5-fold, increase in maximum oxygen consumption rate and/or respiratory reserve capacity.

In some aspects, the methods for generating mature cardiomyocytes from immature cardiomyocytes, further comprise contacting the population of cells comprising immature cardiomyocytes (and optionally endothelial cells) with at least one additional cardiomyocyte maturation factor comprising a p53 activator and/or an inhibitor of mTOR, to induce the maturation (e.g., in vitro maturation) of at least one immature cardiomyocyte in the population into a cardiomyocyte. In some embodiments, a population of cells comprising immature cardiomyocytes is contacted with at least one additional cardiomyocyte maturation factor (e.g., p53 activator and/or mTOR inhibitor). In some aspects, p53 expression is upregulated in combination with mTOR inhibition to enhance the maturation of cardiomyocytes derived from stem cells. In some aspects, p53 expression is upregulated without inhibiting mTOR to enhance the maturation of cardiomyocytes derived from stem cells.

The disclosure contemplates the use of any p53 activator that encourages immature cardiomyocytes to differentiate and/or mature into cardiomyocytes (e.g., alone or in combination with another cardiomyocyte maturation factor (e.g., a FOXO activator, a FOXM1 inhibitor, an mTOR inhibitor)). In some embodiments, the p53 activator is an upregulator of p53 expression. Upregulation of p53 may include an increase in total p53 and phosphorylated p53 protein. Examples of p53 activators or upregulators include small molecule, nucleic acid, amino acid, metabolite, polypeptide, antibody and antibody-like molecules, aptamers, macrocycles, and other molecules. In some embodiments, a p53 activator is selected from the group consisting of MDM2, nutlin-3a, senolytic, quercetin, Torin1, and/or functional and/or structural analogs or variants thereof. In some aspects, an upregulator of p53 is an MDM2 inhibitor. In certain aspects, an upregulator of p53 is nutlin-3a. In one aspect, an upregulator of p53 comprises 5 to 15 μM, 7 to 12 μM or about 10 μM nutlin-3a. In some aspects, an upregulator of p53 is a senolytic. In certain aspects, an upregulator of p53 is quercetin. In some aspects, an upregulator of p53 is Torin1. In some aspects, an upregulator of p53 is an agent that is not Torin1. In some aspects, an upregulator of p53 is an agent that is not an mTOR inhibitor. In some aspects, an upregulator of p53 is a combination of nutlin-3a and Torin1. In some aspects, an upregulator of p53 is a combination of nutlin-3a and quercetin. In some aspects, an upregulator of p53 is a combination of nutlin-3a, quercetin, and Torin1.

In some aspects, the methods for generating mature cardiomyocytes from immature cardiomyocytes, further comprise contacting the population of cells comprising immature cardiomyocytes (and optionally endothelial cells) with at least one additional cardiomyocyte maturation factor comprising an mTOR inhibitor, to induce the maturation (e.g., in vitro maturation) of at least one immature cardiomyocyte in the population into a cardiomyocyte. In some embodiments, the population of cells comprising immature cardiomyocytes is contacted with at least one additional cardiomyocyte maturation factor (e.g., mTOR inhibitor, PI3K inhibitor, or Akt inhibitor). In some aspects, the PI3K/Akt/mTOR pathway is manipulated (e.g., inhibited) to enhance the maturation of cardiomyocytes derived from stem cells.

The disclosure contemplates the use of any mTOR inhibitor that encourages immature cardiomyocytes to differentiate and/or mature into cardiomyocytes (e.g., alone or in combination with another cardiomyocyte maturation factor (e.g., a FOXO activator, a FOXM1 inhibitor, a p53 upregulator)). In some embodiments, mTOR comprises mTORC1 and/or mTORC2. In some embodiments, the mTOR inhibitor is an inhibitor of mTORC1 and/or mTORC2. In some embodiments, the mTOR inhibitor inhibits phosphorylation of 4E-BP1. Inhibiting phosphorylation of 4E-BP1 may affect regulation of the oxidative phosphorylation pathway. Inhibiting phosphorylation of 4E-BP1 may degrade p21 and thereby upregulate p53. Non-limiting examples of modulators of the oxidative phosphorylation pathway include 4EGI-1, JR-AB2-011 (an mTORC2 inhibitor), AICAR (an AMPK activator), metformin (an AMPK activator and mTORC1/2 inhibitor), HLM006474 (an E2F inhibitor), and/or functional and/or structural analogs or variants thereof. In some embodiments, the mTOR inhibitor inhibits phosphorylation of 4E-BP1 and Ribosomal protein S6. In some embodiments, the mTOR inhibitor comprises Torin1, Torin2, rapamycin, everolimus, and/or temsirolimus. In some embodiments, an mTOR inhibitor comprises Torin1. In one embodiment, an mTOR inhibitor comprises 1 to 1500 nM, 5 to 1250 nM, 10 to 1000 nM Torin1. In some embodiments, an mTOR inhibitor comprises Torin2.

In some embodiments, contacting may be performed by maintaining the at least one immature cardiomyocyte or a precursor thereof in culture medium comprising the one or more cardiomyocyte maturation factors. In some embodiments, the contacting is performed by maintaining the at least one immature cardiomyocyte or a precursor thereof in two-dimensional (2D) culture medium comprising the one or more cardiomyocyte maturation factors. In other embodiments, the contacting is performed by maintaining the at least one immature cardiomyocyte or a precursor thereof in three-dimensional (3D) culture medium comprising the one or more cardiomyocyte maturation factors. In some aspects, the one or more cardiomyocyte maturation factors are applied to the culture medium (e.g., the 2D or 3D culture medium) with a pulse treatment. In some embodiments, pulse treatment occurs for 1 to 24 hours, 1 to 18 hours, 1 to 12 hours, 5 to 18 hours, 5 to 12 hours, or 10 to 12 hours. In some embodiments, pulse treatment occurs for 1 hour or longer. In some embodiments, pulse treatment occurs for 24 hours or less. In some embodiments, pulse treatment occurs for a period of 1 to 5 or 2 to 3 days. In one embodiment, a pulse treatment occurs at a pre-determined time and for a pre-determined length of time for 2-3 days, thereby mimicking a circadian cycle. In some aspects, the one or more cardiomyocyte maturation factors are applied to the culture medium (e.g., the 2D or 3D culture medium) with a continuous treatment.

In some embodiments at least one immature cardiomyocyte or a precursor thereof can be genetically engineered. In some embodiments, at least one immature cardiomyocyte or a precursor thereof can be genetically engineered to express one or more cardiomyocyte (e.g., mature cardiomyocyte) markers as disclosed herein, for example express at least one polypeptide selected from TNNI3, TNNT2, Kir2.1, Cx43, and CD36, or an amino acid sequence substantially homologous thereof, or functional fragments or functional variants thereof.

Where the immature cardiomyocytes or precursors thereof are maintained under in vitro conditions, conventional tissue culture conditions and methods can be used, and are known to those of skill in the art. Isolation and culture methods for various cells are well within the abilities of one skilled in the art.

In the methods of the disclosure at least one cardiomyocyte or a precursor thereof can, in general, be cultured under standard conditions of temperature, pH, and other environmental conditions, e.g., as adherent cells in tissue culture plates or in 3D culture in Erlenmeyer flasks at 37° C. in an atmosphere containing 5-10% CO₂. The cells and/or the culture medium are appropriately modified to achieve conversion to cardiomyocytes as described herein.

In certain examples, the cardiomyocyte maturation factors can be used to induce the differentiation of at least one immature cardiomyocyte or precursor thereof by exposing or contacting at least one immature cardiomyocyte or precursor thereof with an effective amount of a cardiomyocyte maturation factor described herein to differentiate the at least one immature cardiomyocyte or precursor thereof into at least one cardiomyocyte (e.g., a mature cardiomyocyte). In some aspects, the exposing or contacting of the immature cardiomyocyte with a cardiomyocyte maturation factor occurs continuously, or in other aspects, occurs via a pulse treatment.

Accordingly, included herein are cells and compositions made by the methods described herein. The exact amount and type of cardiomyocyte maturation factor can vary depending on the number of immature cardiomyocytes or precursors thereof, the desired differentiation stage and the number of prior differentiation stages that have been performed. In certain examples, a cardiomyocyte maturation factor is present in an effective amount. As used herein, “effective amount” refers to the amount of the compound that should be present for the differentiation of at least 10% or at least 20% or at least 30% of the cells in a population of immature cardiomyocytes or precursors thereof into cardiomyocytes.

In additional examples, cardiomyocyte maturation factors can be present in the culture medium of the at least one immature cardiomyocyte or precursor thereof, or alternatively, the cardiomyocyte maturation factors may be added to the at least one immature cardiomyocytes or precursor thereof during some stage of growth.

In some embodiments, immature cardiomyocytes are contacted with a cardiomyocyte maturation factor (e.g., a FOXO activator, a FOXM1 inhibitor, an mTOR inhibitor and/or p53 upregulator) after the immature cardiomyocytes begin beating. In some aspects, immature cardiomyocytes are beating for a period of 1 to 5 days, 1 to 4 days, 1 to 3 days, 1 to 2 days, 1 day, 2 days, 3 days, 4 days, or 5 days before being contacted with a cardiomyocyte maturation factor. In some aspects, immature cardiomyocytes are beating for a period of 1 to 40 days, 2 to 35 days, 3 to 30 days, 4 to 25 days, 5 to 20 days, 7 to 35 days, 14 to 30 days, or 21 to 28 days before being contacted with a cardiomyocyte maturation factor. In some aspects immature cardiomyocytes are not contacted with a cardiomyocyte maturation factor (e.g., a FOXO activator, a FOXM1 inhibitor, an mTOR inhibitor and/or p53 upregulator) if the immature cardiomyocytes have not begun beating.

Where the at least one immature cardiomyocyte or a precursor thereof is maintained under in vitro conditions, conventional tissue culture conditions and methods can be used, and are known to those of skill in the art. Isolation and culture methods for various cells are well within the abilities of one skilled in the art.

In some aspects, pluripotent stem cells are cultured in RPMI+B27 and are contacted with a GSK3 inhibitor/WNT activator (e.g., CHIR 99021) at Day 0 of a differentiation protocol. At Day 2 of the protocol the WNT activator (e.g., CHIR 99021) is removed. At Day 3 of the protocol a WNT inhibitor (e.g., IWP4) is added and at D5 the WNT inhibitor (e.g., IWP4) is removed. At Day 7 insulin is added to the culture and ever 2-3 days the media is changed. At Day 11 a maturation factor is added and is maintained in the culture until Day 18. At the completion of the differentiation protocol, mature cardiomyocytes are obtained from the culture media.

In some aspects, pluripotent stem cells are cultured in RPMI+B27 and are contacted with a GSK3 inhibitor/WNT activator (e.g., CHIR 99021) from days 0 to 2 of the differentiation protocol. From days 2 to 4 of the protocol a WNT inhibitor (e.g., IWP4) is added. At Day 7 insulin is added to the culture and ever 2-3 days the media is changed.

Upon beating, cardiomyocytes were treated with a maturation factor beginning at approximately 2 days after onset of beating for a period of 5-7 days. Upon completion of treatment, media was switched back to RPMI/B27/insulin and maintained with media change every 2-3 days. At the completion of the differentiation protocol, mature cardiomyocytes are obtained from the culture media.

In some embodiments, the differentiation protocol for obtaining cardiomyocytes from immature cardiomyocytes or precursors thereof occurs in a two-dimensional culture system. In some embodiments, the differentiation protocol for obtaining cardiomyocytes from immature cardiomyocytes or precursors thereof occurs in a three-dimensional culture system (e.g., using a 3D bioreactor system).

Aspects of the disclosure involve generating cardiomyocytes which resemble endogenous mature cardiomyocytes in form and function, but nevertheless are distinct from native cardiomyocytes. In some embodiments, the morphology of the cardiomyocytes resembles the morphology of endogenous cardiomyocytes. In some embodiments, the cardiomyocytes are electrically mature. In some embodiments, the cardiomyocytes are contractility mature. In some embodiments, the cardiomyocytes are metabolically mature. In some embodiments, the cardiomyocytes are quiescent. In some embodiments, the cardiomyocytes exhibit increased expression of quiescence markers. In some embodiments, the cardiomyocytes exhibit decreased expression of proliferative markers. In some embodiments, the cardiomyocytes exhibit increased expression of inhibitor E2F factors. In some embodiments, the cardiomyocytes exhibit decreased expression of stimulatory E2F factors.

In some embodiments, the cardiomyocytes are mature. In some embodiments, the cardiomyocytes exhibit increased expression of sarcomeric proteins (e.g., TNNT2 and/or TNNI3). In some embodiments, the cardiomyocytes exhibit decreased beating rate as compared to fetal or immature cardiomyocytes. In some embodiments, the cardiomyocytes exhibit decreased automaticity. In some embodiments, the cardiomyocytes exhibit increased mean beat amplitude, mean spike amplitude, and/or upstroke velocity as compared to immature cardiomyocytes. In some embodiments, the cardiomyocytes exhibit increased oxygen consumption and/or respiratory reserve as compared to immature cardiomyocytes. In some embodiments, the cardiomyocytes exhibit increased expression of one or more markers selected from the group consisting of TNNT2, TNNI3, Kir2.1, Cx43, and CD36 as compared to immature cardiomyocytes.

Generating cardiomyocytes by conversion or maturation of at least one immature cardiomyocyte or a precursor thereof using the methods of the disclosure has a number of advantages. First, the methods of the disclosure allow one to generate autologous cardiomyocytes, which are cell specific to and genetically matched with an individual. In general, autologous cells are less likely than non-autologous cells to be subject to immunological rejection. The cells are derived from at least one immature cardiomyocyte or a precursor thereof, e.g., a cardiac progenitor obtained by reprogramming a somatic cell (e.g., a fibroblast) from the individual to an induced pluripotent state, and then culturing the pluripotent cells to differentiate at least some of the pluripotent cells to at least one immature cardiomyocyte or precursor, followed by the induced maturation in vitro of the at least one immature cardiomyocyte into a cardiomyocyte (e.g., a mature cardiomyocyte).

In some embodiments, a subject from which at least one immature cardiomyocyte or precursor thereof are obtained is a mammalian subject, such as a human subject. In some embodiments, the subject is suffering from a cardiac disorder. In some embodiments, the subject is suffering from chronic heart failure. In some embodiments, the subject is suffering from ventricular arrhythmias. In such embodiments, the at least one immature cardiomyocyte or precursor thereof can be differentiated into a cardiomyocyte ex vivo by the methods as described herein and then administered to the subject from which the cells were harvested in a method to treat the subject for the cardiac disorder (e.g., heart failure).

In some embodiments, at least one immature cardiomyocyte or a precursor thereof is located within a subject (in vivo) and is converted to become a cardiomyocyte by the methods as disclosed herein in vivo. In some embodiments, conversion of at least one immature cardiomyocyte or a precursor thereof to a cardiomyocyte in vivo can be achieved by administering to a subject a composition comprising at least one, at least two, at least three, at least four, or more cardiomyocyte maturation factors as described herein. In some embodiments, conversion of at least one immature cardiomyocyte or a precursor thereof to a cardiomyocyte in vivo can be achieved by administering to a subject a composition comprising at least one, at least two, at least three, or at least four cardiomyocyte maturation factors as described herein.

### Cardiomyocytes

In some embodiments, the disclosure provides mature cardiomyocytes. The cardiomyocytes disclosed herein share many distinguishing features of native cardiomyocytes, but are different in certain aspects (e.g., gene expression profiles). In some embodiments, the cardiomyocyte is non-native or non-naturally occurring. As used herein, “non-native” or “non-naturally occurring” means that the cardiomyocytes are markedly different in certain aspects from cardiomyocytes which exist in nature, i.e., native cardiomyocytes. It should be appreciated, however, that these marked differences typically pertain to structural features which may result in the cardiomyocytes exhibiting certain functional differences, e.g., although the gene expression patterns of cardiomyocytes differs from native cardiomyocytes, the cardiomyocytes behave in a similar manner to native cardiomyocytes but certain functions may be altered (e.g., improved) compared to native cardiomyocytes.

The cardiomyocytes of the disclosure share many characteristic features of native cardiomyocytes which are important for normal cardiomyocyte function. Characteristics of mature cardiomyocytes are described in Yang et al. Circ. Res. 2014; 114 (3): 511-23.

In some embodiments, the cardiomyocytes are quiescent. In some embodiments, cardiomyocytes retain metabolic and transcriptional activity in the quiescent state. In some embodiments, the quiescent state facilitates cardiomyocyte maturation. In some embodiments, cardiomyocytes express, or express at an increased level (i.e., compared to a control) certain quiescent markers.

In some embodiments, the cardiomyocytes are electrically mature cardiomyocytes. In some embodiments, the cardiomyocytes exhibit decreased automaticity. Native mature adult human cardiomyocytes beat at 20-30 beats per minute naturally. In some aspects, the cardiomyocytes described herein exhibit a slower intrinsic beating rate. In some embodiments, the cardiomyocytes beat at 1 to 35 beats per minute, 1 to 30 beats per minute, 1 to 25 beats per minutes, 1 to 20 beats per minute, 1 to 15 beats per minute, 2 to 30 beats per minute, 2 to 25 beats per minute, 2 to 20 beats per minute, 2 to 15 beats per minute, 3 to 30 beats per minute, 3 to 25 beats per minute, 3 to 20 beats per minute, or 3 to 15 beats per minute. In certain embodiments, the cardiomyocytes exhibit a spontaneous beating rate of less than 3 beats per minute. The beating rate of the cardiomyocytes may be dependent or effected by one or more conditions including temperature, pH, age of cells, and the like. In one aspect, a monolayer culture of cardiomyocytes may exhibit a faster beater rate than single cells. Slower intrinsic beating rate may suggest decreased automaticity, and cardiomyocytes with decreased automaticity (i.e., decreased drive to beat spontaneously) may decrease the risk of arrhythmias in cell therapy. In some embodiments, the cardiomyocytes exhibit increased upstroke velocity (spike slope) or mean spike amplitude.

In some embodiments, the cardiomyocytes are contractility mature cardiomyocytes. In some embodiments, the cardiomyocytes exhibit increased RNA and/or protein expression of contractile proteins (e.g., sarcomeric contractile proteins) (i.e., as compared to immature cardiomyocytes). In some aspects, the cardiomyocytes exhibit increased RNA and/or protein expression of at least one of cardiac troponin T (TNNT2) and cardiac troponin I (TNNI3). Increased expression of one or more sarcomeric proteins may enhance contractility of the cardiomyocytes. In some embodiments, the cardiomyocytes exhibit increased mean beat amplitude. In some aspects, a cardiomyocyte has increased contractility as compared to an immature cardiomyocyte.

In some embodiments, the cardiomyocytes are metabolically mature cardiomyocytes. In some aspects, a cardiomyocyte has increased metabolic activity as compared to an immature cardiomyocyte. In some embodiments, a cardiomyocyte has increased oxygen consumption and/or extracellular acidification rate as compared to immature cardiomyocytes. In some embodiments, a cardiomyocyte has increased respiratory reserve as compared to immature cardiomyocytes. Metabolic maturity may be quantified using a Seahorse mito stress metabolic assay (Agilent). The assay may be used to measure oxygen consumption rate and extracellular acidification rate in response to one or more compounds (e.g., small molecule compounds) that affect mitochondrial function.

In some embodiments, the cardiomyocytes exhibit a morphology that resembles the morphology of an endogenous mature cardiomyocyte. In some embodiments, the cardiomyocytes form rod-shaped cells. In some embodiments, the cardiomyocytes exhibit an organized sarcomere structure. In some aspects, the average sarcomere length is 1.0 to 4.0 μm, 1.5 to 3.5 μm, or 2.0 to 3.0 μm.

In some aspects the cardiomyocytes exhibit a mature ion channel expression profile. In some embodiments, the cardiomyocytes exhibit increased ion channel expression (i.e., compared to immature cardiomyocytes). In some embodiments, the cardiomyocytes exhibit increased expression of Kir2.1 (i.e., compared to immature cardiomyocytes). In some embodiments, the cardiomyocytes exhibit increased expression of connexin 43 (Cx43) (i.e., compared to immature cardiomyocytes). In some embodiments, the cardiomyocytes exhibit increased expression of CD36 (i.e., compared to immature cardiomyocytes). In some embodiments, the cardiomyocytes exhibit increased expression of at least one marker selected from the group consisting of TNNT2, TNNI3, Kir2.1, Cx43, and CD36.

The cardiomyocytes are differentiated in vitro from any starting cell as the invention is not intended to be limited by the starting cell from which the cardiomyocytes are derived. Exemplary starting cells include, without limitation, immature cardiomyocytes or any precursor thereof such as a cardiac progenitor cell, a pluripotent stem cell, an embryonic stem cell, and induced pluripotent stem cell. In some embodiments, the cardiomyocytes are differentiated in vitro from a reprogrammed cell, a partially reprogrammed cell (i.e., a somatic cell, e.g., a fibroblast which has been partially reprogrammed such that it exists in an intermediate state between an induced pluripotency cell and the somatic cell from which it has been derived), or a transdifferentiated cell. In some embodiments, the cardiomyocytes disclosed herein can be differentiated in vitro from an immature cardiomyocyte or a precursor thereof. In some embodiments, the cardiomyocyte is differentiated in vitro from a precursor selected from the group consisting of an immature cardiomyocyte, a cardiac progenitor cell, and a pluripotent stem cell. In some embodiments, the pluripotent stem cell is selected from the group consisting of an embryonic stem cell and induced pluripotent stem cell. In some embodiments, the cardiomyocyte or the pluripotent stem cell from which the cardiomyocyte is derived is human. In some embodiments, the cardiomyocyte is human.

In some embodiments, the cardiomyocyte is not genetically modified. In some embodiments, the cardiomyocyte obtains the features it shares in common with native cardiomyocytes in the absence of a genetic modification of cells. In some embodiments, the cardiomyocyte is genetically modified.

In some aspects, the disclosure provides a cell line comprising a cardiomyocyte described herein. In some embodiments, the cardiomyocytes can be frozen, thawed, and passaged. The cardiomyocytes may be passaged at least 5 times without significant morphological changes.

Aspects of the disclosure relate to isolated populations of cardiomyocytes produced according to methods described herein. In some embodiments, a population of cardiomyocytes is produced by contacting at least one immature cardiomyocyte with at least one cardiomyocyte maturation factor described herein. In some embodiments, a population of cardiomyocytes is produced by co-culturing at least one immature cardiomyocyte with at least one endothelial cell. In some embodiments, a population of cardiomyocytes is produced by co-culturing at least one immature cardiomyocyte with at least one endothelial cell, and further contacting the co-culture with at least one cardiomyocyte maturation factor described herein.

Aspects of the disclosure involve microcapsules comprising isolated populations of cells described herein (e.g., cardiomyocytes). Microcapsules are well known in the art. Suitable examples of microcapsules are described in the literature (e.g., Orive et al., “Application of cell encapsulation for controlled delivery of biological therapeutics”, Advanced Drug Delivery Reviews (2013), dx.doi.org/10.1016/j.addr.2013.07.009; Hernandez et al., “Microcapsules and microcarriers for in situ cell delivery”, Advanced Drug Delivery Reviews 2010; 62:711-730; Murua et al., “Cell microencapsulation technology: Towards clinical application”, Journal of Controlled Release 2008; 132:76-83; and Zanin et al., “The development of encapsulated cell technologies as therapies for neurological and sensory diseases”, Journal of Controlled Release 2012; 160:3-13). Microcapsules can be formulated in a variety of ways. Exemplary microcapsules comprise an alginate core surrounded by a polycation layer covered by an outer alginate membrane. The polycation membrane forms a semipermeable membrane, which imparts stability and biocompatibility. Examples of polycations include, without limitation, poly-L-lysine, poly-L-ornithine, chitosan, lactose modified chitosan, and photopolymerized biomaterials. In some embodiments, the alginate core is modified, for example, to produce a scaffold comprising an alginate core having covalently conjugated oligopeptides with an RGD sequence (arginine, glycine, aspartic acid). In some embodiments, the alginate core is modified, for example, to produce a covalently reinforced microcapsule having a chemoenzymatically engineered alginate of enhanced stability. In some embodiments, the alginate core is modified, for example, to produce membrane-mimetic films assembled by in-situ polymerization of acrylate functionalized phospholipids. In some embodiments, microcapsules are composed of enzymatically modified alginates using epimerases. In some embodiments, microcapsules comprise covalent links between adjacent layers of the microcapsule membrane. In some embodiment, the microcapsule comprises a subsieve-size capsule comprising alginate coupled with phenol moieties. In some embodiments, the microcapsule comprises a scaffold comprising alginate-agarose. In some embodiments, the cardiomyocyte is modified with PEG before being encapsulated within alginate. In some embodiments, the isolated populations of cells, e.g., cardiomyocytes are encapsulated in photoreactive liposomes and alginate. It should be appreciated that the alginate employed in the microcapsules can be replaced with other suitable biomaterials, including, without limitation, PEG, chitosan, PES hollow fibers, collagen, hyaluronic acid, dextran with RGD, EHD and PEGDA, PMBV and PVA, PGSAS, agarose, agarose with gelatin, PLGA, and multilayer embodiments of these.

In some embodiments, compositions comprising populations of cardiomyocytes produced according to the methods described herein can also be used as the functional component in a mechanical device. For example, a device may contain a population of cardiomyocytes (e.g., produced from populations of immature cardiomyocytes or precursors thereof) behind a semipermeable membrane that prevents passage of the cell population, retaining them in the device. Other examples of devices include those contemplated for either implantation into a cardiac patient, or for extracorporeal therapy.

Aspects of the disclosure involve assays comprising isolated populations of cardiomyocytes described herein (e.g., mature cardiomyocytes). In some embodiments, the assays can be used for identifying one or more candidate agents which promote or inhibit a mature cardiomyocyte fate. In some embodiments, the assays can be used for identifying one or more candidate agents which promote the differentiation of at least one immature cardiomyocyte or a precursor thereof into cardiomyocytes.

The disclosure contemplates methods in which cardiomyocytes are generated according to the methods described herein from iPS cells derived from cells extracted or isolated from individuals suffering from a disease (e.g., heart failure, or a cardiac-related disorder), and those cardiomyocytes are compared to normal cardiomyocytes from healthy individuals not having the disease to identify differences between the cardiomyocytes and normal cardiomyocytes which could be useful as markers for disease (e.g., epigenetic and/or genetic). In some embodiments, cardiomyocytes are obtained from an individual suffering from heart failure and compared to normal cardiomyocytes, and then the cardiomyocytes are reprogrammed to iPS cells and the iPS cells are analyzed for genetic and/or epigenetic markers which are present in the cardiomyocytes obtained from the individual suffering from heart failure but not present in the normal cardiomyocytes, to identify markers (e.g., pre-heart failure). In some embodiments, the iPS cells and/or cardiomyocytes derived from patients are used to screen for agents (e.g., agents which are able to modulate genes contributing to a heart failure phenotype).

### Confirmation of the Presence and the Identification of Cardiomyocytes

One can use any means common to one of ordinary skill in the art to confirm the presence of a mature cardiomyocyte as compared to the presence of an immature cardiomyocyte. One can use any means common to one of ordinary skill in the art to confirm the presence of a cardiomyocyte, e.g. a mature cardiomyocyte produced by the differentiation of at least one immature cardiomyocyte or precursor thereof by exposure to at least one cardiomyocyte maturation factor as described herein. One can use any means common to one of ordinary skill in the art to confirm the presence of a cardiomyocyte, e.g. a mature cardiomyocyte produced by the differentiation of at least one immature cardiomyocyte or precursor thereof by co-culture with at least one endothelial cell as described herein.

In some embodiments, the presence of cardiomyocyte markers, e.g. chemically induced cardiomyocytes, can be assessed by detecting the presence or absence of one or more markers indicative of an endogenous cardiomyocyte. In some embodiments, the method can include detecting the positive expression (e.g., the presence) of a marker for cardiomyocytes. In some embodiments the method can include detecting the positive expression of one or more sarcomeric proteins (e.g., cardiac troponin T (TNNT2) or cardiac troponin I (TNNI3)). In some embodiments the method can include detecting the positive expression of one or more markers of Kir2.1, Cx43, or CD36. In some embodiments, the marker can be detected using a reagent, e.g., a reagent for the detection of TNNT2, TNNI3, Kir2.1, Cx43, or CD36. Cardiomyocytes can also be characterized by the down-regulation of specific markers.

A reagent for a marker can be, for example, an antibody against the marker or primers for a RT-PCR or PCR reaction, e.g., a semi-quantitative or quantitative RT-PCR or PCR reaction. Such markers can be used to evaluate whether a cardiomyocyte has been produced. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

The progression of at least one immature cardiomyocyte to a mature state can be monitored by determining the expression of markers characteristic of mature cardiomyocytes. In some processes, the expression of certain markers is determined by detecting the presence or absence of the marker. Alternatively, the expression of certain markers can be determined by measuring the level at which the marker is present in the cells of the cell culture or cell population. In certain processes, the expression of markers characteristic of mature cardiomyocytes as well as the lack of significant expression of markers characteristic of immature cardiomyocytes from which it was derived is determined.

The progression of at least one immature cardiomyocyte or precursor thereof to a cardiomyocyte can be monitored by determining the expression of markers characteristic of mature cardiomyocytes. In some processes, the expression of certain markers is determined by detecting the presence or absence of the marker. Alternatively, the expression of certain markers can be determined by measuring the level at which the marker is present in the cells of the cell culture or cell population. In certain processes, the expression of markers characteristic of mature cardiomyocytes as well as the lack of significant expression of markers characteristic of immature cardiomyocytes or precursors thereof from which it was derived is determined.

As described in connection with monitoring the production of a cardiomyocyte (e.g., a mature cardiomyocyte) from an immature cardiomyocyte, qualitative or semi-quantitative techniques, such as blot transfer methods and immunocytochemistry, can be used to measure marker expression, using methods commonly known to persons of ordinary skill in the art. Alternatively, marker expression can be accurately quantitated through the use of techniques such as quantitative-PCR by methods ordinarily known in the art. Additionally, techniques for measuring extracellular marker content, such as ELISA, may be utilized.

It is understood that the present invention is not limited to those markers listed as cardiomyocyte markers herein, and the present invention also encompasses markers such as cell surface markers, antigens, and other gene products including ESTs, RNA (including microRNAs and antisense RNA), DNA (including genes and cDNAs), and portions thereof.

### Enrichment, Isolation and Purification of a Cardiomyocyte

Another aspect of the present invention relates to the isolation of a population of cardiomyocytes (e.g., mature cardiomyocytes) from a heterogeneous population of cells, such as a mixed population of cells comprising mature cardiomyocytes and immature cardiomyocytes or precursors thereof from which the mature cardiomyocyte was derived. In some aspects, a population of cardiomyocytes are isolated from a heterogenous population of cells, such as a mixed population of cells comprising immature cardiomyocytes and mature cardiomyocytes. A population of cardiomyocytes produced by any of the above-described processes can be enriched, isolated and/or purified by using any cell surface marker present on the cardiomyocyte which is not present on the immature cardiomyocyte or precursor thereof from which it was derived. Such cell surface markers are also referred to as an affinity tag which is specific for a cardiomyocyte (e.g., a mature cardiomyocyte). Examples of affinity tags specific for cardiomyocytes are antibodies, ligands or other binding agents that are specific to a marker molecule, such as a polypeptide, that is present on the cell surface of a cardiomyocyte but which is not substantially present on other cell types (e.g. immature cardiomyocytes). In some processes, an antibody which binds to a cell surface antigen on a cardiomyocyte is used as an affinity tag for the enrichment, isolation or purification of chemically induced cardiomyocytes produced by the methods described herein. Such antibodies are known and commercially available.

The skilled artisan will readily appreciate the processes for using antibodies for the enrichment, isolation and/or purification of cardiomyocytes. For example, in some embodiments, the reagent, such as an antibody, is incubated with a cell population comprising cardiomyocytes, wherein the cell population has been treated to reduce intercellular and substrate adhesion. The cell population is then washed, centrifuged and resuspended. In some embodiments, if the antibody is not already labeled with a label, the cell suspension is then incubated with a secondary antibody, such as an FITC-conjugated antibody that is capable of binding to the primary antibody. The cardiomyocytes are then washed, centrifuged and resuspended in buffer. The cardiomyocyte suspension is then analyzed and sorted using a fluorescence activated cell sorter (FACS). Antibody-bound, fluorescent reprogrammed cells are collected separately from non-bound, non-fluorescent cells, thereby resulting in the isolation of cardiomyocytes from other cells present in the cell suspension, e.g., immature cardiomyocytes or precursors thereof.

In another embodiment of the processes described herein, the isolated cell composition comprising cardiomyocytes can be further purified by using an alternate affinity-based method or by additional rounds of sorting using the same or different markers that are specific for cardiomyocytes. For example, in some embodiments, FACS sorting is used to first isolate a cardiomyocyte which expresses TNNT2. In some aspects, TNNI3 is used as a marker for FACS sorting, either alone or in combination with TNNT2. In some aspects, Kir2.1, CD36, and/or Cx43 are used as a marker(s) for FACS sorting, either alone or in combination with TNNT2 and/or TNNI3. A second FACS sorting, e.g. sorting the positive cells again using FACS to isolate cells that are positive for a different marker than the first sort enriches the cell population for reprogrammed cells.

In an alternative embodiment, FACS sorting is used to separate cells by negatively sorting for a marker that is present on most immature cardiomyocytes but is not present on cardiomyocytes (e.g., mature cardiomyocytes).

In some embodiments of the processes described herein, cardiomyocytes are fluorescently labeled without the use of an antibody then isolated from non-labeled cells by using a fluorescence activated cell sorter (FACS). In such embodiments, a nucleic acid encoding GFP, YFP or another nucleic acid encoding an expressible fluorescent marker gene, such as the gene encoding luciferase, is used to label reprogrammed cells using the methods described above.

In addition to the procedures just described, chemically induced cardiomyocytes may also be isolated by other techniques for cell isolation. Additionally, cardiomyocytes may also be enriched or isolated by methods of serial subculture in growth conditions which promote the selective survival or selective expansion of the cardiomyocytes. Such methods are known by persons of ordinary skill in the art.

Using the methods described herein, enriched, isolated and/or purified populations of cardiomyocytes can be produced in vitro from immature cardiomyocytes or precursors thereof (which were differentiated from pluripotent stem cells by the methods described herein). In some embodiments, preferred enrichment, isolation and/or purification methods relate to the in vitro production of human cardiomyocytes from human immature cardiomyocytes or precursors thereof, which were differentiated from human pluripotent stem cells, or from human induced pluripotent stem (iPS) cells. In such an embodiment, where cardiomyocytes are differentiated from immature cardiomyocytes, which were previously derived from iPS cells, the cardiomyocytes can be autologous to the subject from whom the cells were obtained to generate the iPS cells.

Using the methods described herein, isolated cell populations of cardiomyocytes are enriched in cardiomyocyte (e.g., mature cardiomyocyte) content by at least about 1-to about 1000-fold as compared to a population of cells before the chemical induction of the immature cardiomyocyte or precursor population. In some embodiments the population of cardiomyocytes is induced, enhances, enriched, or increased by at least 5%, 10%, 20%, 30%, 40%, 50%, 50%, 70%, 80%, 90%, 1-fold, 1.1-fold, 1.5-fold, 2-fold, 3-fold, 4-fold, 5-fold, 10-fold, 50-fold, 100-fold or more as compared to a population of cells before the chemical induction of immature cardiomyocyte or precursor population.

### Compositions Comprising Cardiomyocytes

Some embodiments of the present invention relate to cell compositions, such as cell cultures or cell populations, comprising cardiomyocytes, wherein the cardiomyocytes have been derived from at least one immature cardiomyocyte. In some embodiments, the cell compositions comprise immature cardiomyocytes.

In accordance with certain embodiments, the chemically induced cardiomyocytes are mammalian cells, and in a preferred embodiment, such cardiomyocytes are human cardiomyocytes. In some embodiments, the immature cardiomyocytes have been derived from pluripotent stem cells (e.g., human pluripotent stem cells).

In some embodiments, the cell compositions comprise mature cardiomyocytes, wherein the mature cardiomyocytes have been derived from at least one immature cardiomyocyte using methods described herein. For example, the cell compositions comprise mature cardiomyocytes obtained from the culturing of immature cardiomyocytes in 2D or 3D culture, wherein the immature cardiomyocytes were contacted with one or more cardiomyocyte maturation factors.

Other embodiments of the present invention relate to compositions, such as an isolated cell population or cell culture, comprising cardiomyocytes produced by the methods as disclosed herein. In such embodiments, the cardiomyocytes comprise less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 12%, less than about 10%, less than about 8%, less than about 6%, less than about 5%, less than about 4%, less than about 3%, less than about 2% or less than about 1% of the total cells in the cardiomyocyte population. In some embodiments, the composition comprises a population of cardiomyocytes which make up more than about 90% of the total cells in the cell population, for example about at least 95%, or at least 96%, or at least 97%, or at least 98% or at least about 99%, or about at least 100% of the total cells in the cell population are cardiomyocytes.

Certain other embodiments of the present invention relate to compositions, such as an isolated cell population or cell cultures, comprising a combination of cardiomyocytes (e.g., mature cardiomyocytes) and immature cardiomyocytes or precursors thereof from which the cardiomyocytes were derived. In some embodiments, the immature cardiomyocytes from which the cardiomyocytes are derived comprise less than about 25%, less than about 20%, less than about 15%, less than about 10%, less than about 5%, less than about 4%, less than about 3%, less than about 2% or less than about 1% of the total cells in the isolated cell population or culture.

Additional embodiments of the present invention relate to compositions, such as isolated cell populations or cell cultures, produced by the processes described herein and which comprise chemically induced cardiomyocytes as the majority cell type. In some embodiments, the methods and processes described herein produce an isolated cell culture and/or cell populations comprising at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at least about 85%, at least about 84%, at least about 83%, at least about 82%, at least about 81%, at least about 80%, at least about 79%, at least about 78%, at least about 77%, at least about 76%, at least about 75%, at least about 74%, at least about 73%, at least about 72%, at least about 71%, at least about 70%, at least about 69%, at least about 68%, at least about 67%, at least about 66%, at least about 65%, at least about 64%, at least about 63%, at least about 62%, at least about 61%, at least about 60%, at least about 59%, at least about 58%, at least about 57%, at least about 56%, at least about 55%, at least about 54%, at least about 53%, at least about 52%, at least about 51% or at least about 50% cardiomyocytes.

In another embodiment, isolated cell populations or compositions of cells (or cell cultures) comprise human cardiomyocytes. In other embodiments, the methods and processes as described herein can produce isolated cell populations comprising at least about 50%, at least about 45%, at least about 40%, at least about 35%, at least about 30%, at least about 25%, at least about 24%, at least about 23%, at least about 22%, at least about 21%, at least about 20%, at least about 19%, at least about 18%, at least about 17%, at least about 16%, at least about 15%, at least about 14%, at least about 13%, at least about 12%, at least about 11%, at least about 10%, at least about 9%, at least about 8%, at least about 7%, at least about 6%, at least about 5%, at least about 4%, at least about 3%, at least about 2% or at least about 1% cardiomyocytes. In preferred embodiments, isolated cell populations can comprise human cardiomyocytes. In some embodiments, the percentage of cardiomyocytes in the cell cultures or populations is calculated without regard to the feeder cells remaining in the culture.

Still other embodiments of the present invention relate to compositions, such as isolated cell populations or cell cultures, comprising mixtures of cardiomyocytes and immature cardiomyocytes or precursors thereof from which they were differentiated or matured from. For example, cell cultures or cell populations comprising at least about 5 cardiomyocytes for about every 95 immature cardiomyocytes or precursors thereof can be produced. In other embodiments, cell cultures or cell populations comprising at least about 95 cardiomyocytes for about every 5 immature cardiomyocytes or precursors thereof can be produced. Additionally, cell cultures or cell populations comprising other ratios of cardiomyocytes to immature cardiomyocytes or precursors thereof are contemplated. For example, compositions comprising at least about 1 cardiomyocyte for about every 1,000,000, or at least 100,000 cells, or at least 10,000 cells, or at least 1000 cells or 500, or at least 250 or at least 100 or at least 10 immature cardiomyocytes or precursors thereof can be produced.

Further embodiments of the present invention relate to compositions, such as cell cultures or cell populations, comprising human cells, including human cardiomyocytes, which displays at least one characteristic of an endogenous cardiomyocyte.

In preferred embodiments of the present invention, cell cultures and/or cell populations of cardiomyocytes comprise human cardiomyocytes that are non-recombinant cells. In such embodiments, the cell cultures and/or cell populations are devoid of or substantially free of recombinant human cardiomyocytes.

### Cardiomyocyte Maturation Factors

Aspects of the disclosure involve contacting immature cardiomyocytes or precursors thereof with cardiomyocyte maturation factors, for example, to induce the maturation of the immature cardiomyocytes or differentiation of the precursors thereof into cardiomyocytes (e.g., mature cardiomyocytes). The term “cardiomyocyte maturation factor” refers to an agent that promotes or contributes to the conversion of at least one immature cardiomyocyte or a precursor thereof to a cardiomyocyte. In some embodiments, the cardiomyocyte maturation factor induces the differentiation of pluripotent cells (e.g., iPSCs or hESCs) into immature cardiomyocytes, e.g., in accordance with a method described herein. In some embodiments, the cardiomyocyte maturation factor induces the maturation of immature cardiomyocytes into cardiomyocytes, e.g., in accordance with a method described herein. In some embodiments, a cardiomyocyte maturation factor induces a senescent cardiomyocyte to transition to a quiescent cardiomyocyte.

Generally, at least one cardiomyocyte maturation factor described herein can be used alone, or in combination with other cardiomyocyte maturation factors, to generate cardiomyocytes according to the methods as disclosed herein. In some embodiments, at least two, at least three, at least four, at least five, at least six, at least seven, at least eight, at least nine, or at least ten cardiomyocyte maturation factors described herein are used in the methods of generating cardiomyocytes (e.g., mature cardiomyocytes).

In some embodiments, a cardiomyocyte maturation factor comprises a small molecule, nucleic acid, amino acid, metabolite, polypeptide, antibody and antibody-like molecules, aptamers, macrocycles, or other molecules.

In some embodiments, a cardiomyocyte maturation factor comprises a modulator (e.g., activator) of FOXO. In some embodiments, a cardiomyocyte maturation factor comprises a modulator (e.g., inhibitor) of FOXM1. In some embodiments, a cardiomyocyte maturation factor comprises a regulator of FOXO-FOXM1 signaling. In some embodiments, a cardiomyocyte maturation factor is selected from the group consisting of RCM1, FDI-6, thiostrepton, honokiol, siomycin A, curcumin, bortezomib, MG115, MG132, LOM612, Torin2, metformin, resveratrol, Selinexor, celecoxib, doxorubicin, carbenoxolone, and combinations thereof. In some embodiments, a cardiomyocyte maturation factor comprises an activator of FOXO and is selected from the group consisting of LOM612, Torin2, metformin, resveratrol, Selinexor, celecoxib, doxorubicin, carbenoxolone, and combinations thereof. In some embodiments, a cardiomyocyte maturation factor comprises an inhibitor of FOXM1 and is selected from the group consisting of RCM1, FDI-6, thiostrepton, honokiol, siomycin A, curcumin, bortezomib, MG115, MG132, and combinations thereof. In some embodiments, FOXO may be activated by low glucose and low insulin culturing conditions. Additional activators of FOXO are described in Calissi et al., Nat Rev Drug Discov., 20 (1): 21-38 (2021), which is incorporated herein by reference. Additional inhibitors of FOXM1 are described in Gartel, “Expert Opin Investig Drugs, 19 (2): 235-242 (2010); Chesnokov et al., Cell Death & Disease, 12:704 (2021; and Bhat et al., PLOS ONE, 4 (8): e6593 (2009), each of which is incorporated herein by reference. In certain aspects, a cardiomyocyte maturation factor is selected from the group consisting of RCM1, LOM612, and combinations thereof. In one embodiment, a cardiomyocyte maturation factor comprises RCM1. In one embodiment, a cardiomyocyte maturation factor comprises LOM612.

In some embodiments, a cardiomyocyte maturation factor comprises a modulator (e.g., inhibitor) of the phosphoinositide 3-kinase (PI3K)/Akt/mTOR pathway. In some embodiments, a cardiomyocyte maturation factor comprises an inhibitor of the mTOR pathway. In some embodiments, a cardiomyocyte maturation factor comprises an inhibitor of PI3K and/or Akt. In some embodiments, a cardiomyocyte maturation factor is selected from the group consisting of Torin1, Torin2, rapamycin, everolimus, and temsirolimus. In some embodiments, a cardiomyocyte maturation factor is Torin1. In some embodiments, a cardiomyocyte maturation factor is Torin2. In some embodiments, a cardiomyocyte maturation factor is rapamycin. In some embodiments, a cardiomyocyte maturation factor is everolimus. In some embodiments, a cardiomyocyte maturation factor is temsirolimus.

In some embodiments, a cardiomyocyte maturation factor comprises a modulator of senescent cells. For example, a modulator of senescent cells may be a senolytic. In some embodiments, a cardiomyocyte maturation factor reduces, and in certain aspects eliminates, senescent cells. In some embodiments, a cardiomyocyte maturation factor is selected from the group consisting of fisetin, luteolin, curcumin, geldanamycin, tanespimycin, alvespimyycin, piperlongumine, FOXO4-related peptide, nutlin-3a, ouabain, proscillaridin A, digoxin, quercetin, dasatinib, navitoclax, and combinations thereof. In certain embodiments, a cardiomyocyte maturation factor is quercetin. In some aspects, quercetin increases expression of p53 and/or Kir2.1.

In some embodiments, a cardiomyocyte maturation factor comprises a modulator (e.g., upregulator) of the cell cycle regulator p53. In some embodiments, a cardiomyocyte maturation factor comprises an upregulator or activator of p53. In some embodiments, a cardiomyocyte maturation factor comprises a small molecule, nucleic acid, amino acid, metabolite, polypeptide, antibody and antibody-like molecules, aptamers, macrocycles, or other molecules. In some embodiments, a cardiomyocyte maturation factor is an MDM2 inhibitor. In some embodiments, a cardiomyocyte maturation factor is selected from the group consisting of RG7112, idasanutlin, AMG-232, APG-115, BI-907828, CGM097, siremadlin, milademetan, nutlin-3a, and combinations thereof. In one embodiment, a cardiomyocyte maturation factor is an MDM2 inhibitor (e.g., nutlin-3a). In one embodiment, a cardiomyocyte maturation actor is a senolytic (e.g., quercetin).

In some embodiments, a cardiomyocyte maturation factor is selected from the group consisting of Torin1, nutlin-3a, and quercetin. In some embodiments, a cardiomyocyte maturation factor is Torin1. In some embodiments, a cardiomyocyte maturation factor in nutlin-3a. In some embodiments, a cardiomyocyte maturation factor is quercetin. In some embodiments, p53 is upregulated (e.g., synergistically) by administering one or more of nutlin-3a, quercetin, and Torin1. In some embodiments, a cardiomyocyte maturation factor is not Torin1. In some embodiments, a cardiomyocyte maturation factor is not an mTOR inhibitor.

### Compositions and Kits

Described herein are compositions which comprise a cardiomyocyte described herein (e.g., a mature cardiomyocyte). In some embodiments, the composition also includes a cardiomyocyte maturation factor described herein and/or cell culture media. Described herein are also compositions comprising the compounds described herein (e.g., cell culture media comprising one or more of the compounds described herein or cell culture comprising endothelial cells).

Also described herein are kits for practicing methods disclosed herein and for making cardiomyocytes (e.g., mature cardiomyocytes) disclosed herein. Also described herein are kits for treating chronic heart failure and reducing the incidence of ventricular arrhythmias. In one aspect, a kit includes at least one immature cardiomyocyte or precursor thereof and at least one maturation factor as described herein, and optionally, the kit can further comprise instructions for converting at least one immature cardiomyocyte or precursor thereof to a population of mature cardiomyocytes using a method described herein (e.g., using 2D or 3D culture). In some embodiments, the kit comprises at least two maturation factors. In some embodiments, the kit comprises at least three maturation factors. In some embodiments, the kit comprises any combination of maturation factors.

In some embodiment, the compound in the kit can be provided in a watertight or gas tight container which in some embodiments is substantially free of other components of the kit. The compound can be supplied in more than one container, e.g., it can be supplied in a container having sufficient reagent for a predetermined number of reactions e.g., 1, 2, 3 or greater number of separate reactions to induce immature cardiomyocytes, or precursors thereof, into mature cardiomyocytes. A maturation factor can be provided in any form, e.g., liquid, dried or lyophilized form. It is preferred that a compound(s) (e.g., maturation factor) described herein be substantially pure and/or sterile. When a compound(s) described herein is provided in a liquid solution, the liquid solution preferably is an aqueous solution, with a sterile aqueous solution being preferred. When a compound(s) described herein is provided as a dried form, reconstitution generally is by the addition of a suitable solvent. The solvent, e.g., sterile water or buffer, can optionally be provided in the kit.

In some embodiments, the kit further optionally comprises informational material. The informational material can be descriptive, instructional, marketing or other material that relates to the methods described herein and/or the use of a compound(s) described herein for the methods described herein.

The informational material of the kits is not limited in its instruction or informative material. In one embodiment, the informational material can include information about production of the compound, molecular weight of the compound, concentration, date of expiration, batch or production site information, and so forth. In one embodiment, the informational material relates to methods for administering the compound. Additionally, the informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In one embodiment, the informational material can include instructions to administer a compound(s) (e.g., a maturation factor) as described herein in a suitable manner to perform the methods described herein, e.g., in a suitable dose, dosage form, or mode of administration (e.g., a dose, dosage form, or mode of administration described herein) (e.g., to a cell in vitro or a cell in vivo). In another embodiment, the informational material can include instructions to administer a compound(s) described herein to a suitable subject, e.g., a human, e.g., a human having or at risk for a disorder described herein or to a cell in vitro.

In addition to a compound(s) described herein, the composition of the kit can include other ingredients, such as a solvent or buffer, a stabilizer, a preservative, a flavoring agent (e.g., a bitter antagonist or a sweetener), a fragrance or other cosmetic ingredient, and/or an additional agent for treating a condition or disorder described herein. Alternatively, the other ingredients can be included in the kit, but in different compositions or containers than a compound described herein. In such embodiments, the kit can include instructions for admixing a compound(s) described herein and the other ingredients, or for using a compound(s) described herein together with the other ingredients, e.g., instructions on combining the two agents prior to administration.

The kit can include one or more containers for the composition containing at least one maturation factor as described herein. In some embodiments, the kit contains separate containers (e.g., two separate containers for the two agents), dividers or compartments for the composition(s) and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

The kit can also include a component for the detection of a marker for cardiomyocytes, e.g., for a marker described herein, e.g., a reagent for the detection of mature cardiomyocytes. Or in some embodiments, the kit can also comprise reagents for the detection of negative markers of cardiomyocytes for the purposes of negative selection of mature cardiomyocytes or for identification of cells which do not express these negative markers (e.g., cardiomyocytes). The reagents can be, for example, an antibody against the marker or primers for a RT-PCR or PCR reaction, e.g., a semi-quantitative or quantitative RT-PCR or PCR reaction. Such markers can be used to evaluate whether an iPS cell has been produced. If the detection reagent is an antibody, it can be supplied in dry preparation, e.g., lyophilized, or in a solution. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

The kit can include cardiomyocytes, e.g., mature cardiomyocytes derived from the same type of immature cardiomyocyte or precursor thereof, for example for the use as a positive cell type control.

### Methods of Administering a Cell

In one embodiment, the cells described herein, e.g., a population of mature cardiomyocytes are transplantable, e.g., a population of cardiomyocytes can be administered to a subject. In some embodiments, the cells described herein, e.g., a population of mature cardiomyocytes are transplantable, e.g., a population of cardiomyocytes can be administered to a subject. In some embodiments, the subject who is administered a population of cardiomyocytes is the same subject from whom a pluripotent stem cell used to differentiate into a cardiomyocyte was obtained (e.g. for autologous cell therapy). In some embodiments, the subject is a different subject. In some embodiments, a subject is suffering from chronic heart failure, or is a normal subject. For example, the cells for transplantation (e.g., a composition comprising a population of cardiomyocytes) can be a form suitable for transplantation.

The method can further include administering the cells to a subject in need thereof, e.g., a mammalian subject, e.g., a human subject. The source of the cells can be a mammal, preferably a human. The source or recipient of the cells can also be a non-human subject, e.g., an animal model. The term “mammal” includes organisms, which include mice, rats, cows, sheep, pigs, rabbits, goats, horses, monkeys, dogs, cats, and preferably humans. Likewise, transplantable cells can be obtained from any of these organisms, including a non-human transgenic organism. In one embodiment, the transplantable cells are genetically engineered, e.g., the cells include an exogenous gene or have been genetically engineered to inactivate or alter an endogenous gene.

A composition comprising a population of cardiomyocytes can be administered to a subject using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22 (6): 563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

### Pharmaceutical Compositions

For administration to a subject, a cell population produced by the methods as disclosed herein, e.g. a population of cardiomyocytes (produced by contacting at least one immature cardiomyocyte with at least one maturation factor (e.g., any one, two, three, or more maturation factors as described herein) or by contacting at least one immature cardiomyocyte with at least one endothelial cell as described herein) can be administered to a subject, for example in pharmaceutically acceptable compositions. These pharmaceutically acceptable compositions comprise a therapeutically-effective amount of a population of mature cardiomyocytes as described above, formulated together with one or more pharmaceutically acceptable carriers (additives) and/or diluents. In some aspects, the pharmaceutically acceptable compositions comprise a therapeutically-effective amount of a population of mature cardiomyocytes as described above, formulated together with one or more pharmaceutically acceptable carriers (additives) and/or diluents.

As described in detail below, the pharmaceutical compositions of the present invention can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. Sec, for example, Urquhart, et al., Ann. Rev. Pharmacol. Toxicol. 24:199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL and LDL; (24) C₂-C₁₂ alcohols, such as ethanol; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The phrase “therapeutically-effective amount” as used herein in respect to a population of cells means that amount of relevant cells in a population of cells, e.g., mature cardiomyocytes, or composition comprising mature cardiomyocytes of the present invention which is effective for producing some desired therapeutic effect in at least a sub-population of cells in an animal at a reasonable benefit/risk ratio applicable to any medical treatment. For example, an amount of a population of mature cardiomyocytes administered to a subject that is sufficient to produce a statistically significant, measurable change in at least one symptom of chronic heart failure, such as systolic heart function or incidence of ventricular arrhythmias, etc. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

By “treatment”, “prevention” or “amelioration” of a disease or disorder is meant delaying or preventing the onset of such a disease or disorder, reversing, alleviating, ameliorating, inhibiting, slowing down or stopping the progression or severity of a condition associated with such a disease or disorder. In one embodiment, the symptoms of a disease or disorder are alleviated by at least 5%, at least 10%, at least 20%, at least 30%, at least 40%, or at least 50%.

As used herein, the term “administer” refers to the placement of a composition into a subject by a method or route which results in at least partial localization of the composition at a desired site such that the desired effect is produced. Routes of administration suitable for the methods of the invention include both local and systemic administration. Generally, local administration results in more of the administered cardiomyocytes being delivered to a specific location as compared to the entire body of the subject, whereas systemic administration results in delivery of the cardiomyocytes to essentially the entire body of the subject.

In the context of administering a compound treated cell, the term “administering” also include transplantation of such a cell in a subject. As used herein, the term “transplantation” refers to the process of implanting or transferring at least one cell to a subject. The term “transplantation” includes, e.g., autotransplantation (removal and transfer of cell(s) from one location on a patient to the same or another location on the same patient), allotransplantation (transplantation between members of the same species), and xenotransplantation (transplantation between members of different species).

Mature cardiomyocytes or compositions comprising the same can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, ingestion, or topical application. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection. In other preferred embodiments, the compositions are administered via a cell patch. In some embodiments, the compositions are administered via a three-dimensional structure (e.g., a matrix or scaffold). In some embodiments, the compositions are administered via a micro-tissue.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. The terms, “patient” and “subject” are used interchangeably herein. A subject can be male or female.

Preferably, the subject is a mammal. The mammal can be a human, non-human primate, mouse, rat, dog, cat, horse, or cow, but are not limited to these examples. Mammals other than humans can be advantageously used as subjects that represent animal models of disorders associated with decreased systolic heart function or ventricular arrhythmias. In addition, the methods and compositions described herein can be used to treat domesticated animals and/or pets.

A subject can be one who has been previously diagnosed with or identified as suffering from or having a disorder characterized with decreased systolic heart function or ventricular arrhythmias. A subject may be someone who has been previously diagnosed with or identified as having heart failure (e.g., chronic heart failure). In some aspects, a subject may be someone who has been previously diagnosed with or identified as having a cardiac-related disease or disorder. In some aspects, a subject may be someone who has been previously diagnosed with congenital heart disease (e.g., systolic heart disease or heart disease as a result of tissue engineering).

In some embodiments of the aspects described herein, the method further comprises diagnosing and/or selecting a subject for decreased systolic heart function or ventricular arrhythmias before treating the subject. In some aspects, the method further comprises diagnosing and/or selecting a subject for a cardiac-related disease or disorder before treating the subject. In some aspects, the method further comprises diagnosing and/or selecting a subject for congenital heart disease before treating the subject.

A cardiomyocyte composition described herein can be administered in combination with a mechanical support device (e.g., ventricular assist devices (VADs) or extracorporeal membrane oxygenation (ECMO) systems used to support ventricular recovery), or in combination with cardiac catheterization procedures to revascularize the heart (e.g., stent placement or balloon angioplasty of coronary arteries, or surgical bypass grafting). A cardiomyocyte composition described herein can be co-administrated to a subject in combination with a pharmaceutically active agent. Exemplary pharmaceutically active compound include, but are not limited to, those found in Harrison's Principles of Internal Medicine, 13^{th} Edition, Eds. T. R. Harrison et al. McGraw-Hill N.Y., NY; Physicians' Desk Reference, 50^{th} Edition, 1997, Oradell New Jersey, Medical Economics Co.; Pharmacological Basis of Therapeutics, 8^{th} Edition, Goodman and Gilman, 1990; United States Pharmacopcia, The National Formulary, USP XII NF XVII, 1990; current edition of Goodman and Oilman's The Pharmacological Basis of Therapeutics; and current edition of The Merck Index, the complete content of all of which are herein incorporated in its entirety.

The composition comprising cardiomyocytes and/or a pharmaceutically active agent can be administrated to the subject in the same pharmaceutical composition or in different pharmaceutical compositions (at the same time or at different times). When administrated at different times, the composition comprising cardiomyocytes and/or the pharmaceutically active agent can be administered within 5 minutes, 10 minutes, 20 minutes, 60 minutes, 2 hours, 3 hours, 4, hours, 8 hours, 12 hours, 24 hours of administration of the other. When the composition comprising cardiomyocytes and/or the pharmaceutically active agent are administered in different pharmaceutical compositions, routes of administration can be different. In some embodiments, a subject is administered a composition comprising cardiomyocytes. In other embodiments, a subject is administered a composition comprising a pharmaceutically active agent. In another embodiment, a subject is administered a composition comprising a population of cardiomyocytes mixed with a pharmaceutically active agent. In another embodiment, a subject is administered a composition comprising a population of cardiomyocytes and a composition comprising a pharmaceutically active agent, where administration is substantially at the same time, or subsequent to each other.

Toxicity and therapeutic efficacy of administration of compositions comprising a population of cardiomyocytes can be determined by standard pharmaceutical procedures in cell cultures or experimental animals, e.g., for determining the LD50 (the dose lethal to 50% of the population) and the ED50 (the dose therapeutically effective in 50% of the population). Compositions comprising a population of cardiomyocytes that exhibit large therapeutic indices are preferred.

The amount of a composition comprising a population of cardiomyocytes can be tested using several well-established animal models.

In some embodiments, data obtained from the cell culture assays and in animal studies can be used in formulating a range of dosages for use in humans. The dosage of such compounds lies preferably within a range of circulating concentrations that include the ED50 with little or no toxicity. The dosage may vary within this range depending upon the dosage form employed and the route of administration utilized.

The therapeutically effective dose of a composition comprising a population of cardiomyocytes can also be estimated initially from cell culture assays. Alternatively, the effects of any particular dosage can be monitored by a suitable bioassay.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alterations to a treatment regimen. The dosing schedule can vary from once a week to daily depending on a number of clinical factors. The desired dose can be administered at one time or divided into subdoses, e.g., 2-4 subdoses and administered over a period of time, e.g., at appropriate intervals through the day or other appropriate schedules. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

In another aspect of the invention, the methods provide use of an isolated population of cardiomyocytes as disclosed herein. In one embodiment of the invention, an isolated population of cardiomyocytes as disclosed herein may be used for the production of a pharmaceutical composition, for the use in transplantation into subjects in need of treatment, e.g. a subject that has, or is at risk of developing a ventricular arrhythmia or decreased systolic heart function (e.g., chronic heart failure). In one embodiment, an isolated population of cardiomyocytes may be genetically modified. In another aspect, the subject may have or be at risk of ventricular arrhythmias or decreased systolic heart function. In some embodiments, an isolated population of cardiomyocytes as disclosed herein may be autologous and/or allogeneic. In some embodiments, the subject is a mammal, and in other embodiments the mammal is a human.

One embodiment of the invention relates to a method of treating chronic heart failure in a subject comprising administering an effective amount of a composition comprising a population of cardiomyocytes as disclosed herein to a subject with chronic heart failure. Other embodiments relate to a method of treating a ventricular arrhythmia in a subject comprising administering an effective amount of a composition comprising a population of cardiomyocytes as disclosed herein to a subject with a ventricular arrhythmia. In a further embodiment, the invention provides a method for treating decreased systolic heart function, comprising administering a composition comprising a population of cardiomyocytes as disclosed herein to a subject with decreased systolic heart function. In another embodiment, the invention provides a method for treating congenital heart disease comprising administering an effective amount of a composition comprising a population of cardiomyocytes as disclosed herein to a subject with congenital heart disease.

In some embodiments, a population of cardiomyocytes as disclosed herein may be administered in any physiologically acceptable excipient, where the cardiomyocytes may find an appropriate site for replication, proliferation, and/or engraftment. In some embodiments, a population of cardiomyocytes as disclosed herein can be introduced by injection, catheter, or the like. In some embodiments, a population of cardiomyocytes as disclosed herein can be frozen at liquid nitrogen temperatures and stored for long periods of time, and is capable of use on thawing. If frozen, a population of cardiomyocytes will usually be stored in a 10% DMSO, 50% FCS, 40% RPMI 1640 medium or other cryoprotective solution. Once thawed, the cells may be expanded by use of growth factors and/or feeder cells associated with culturing cardiomyocytes as disclosed herein.

In some embodiments, a population of cardiomyocytes as disclosed herein can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition comprising a population of cardiomyocytes as disclosed herein will be adapted in accordance with the route and device used for administration. In some embodiments, a composition comprising a population of cardiomyocytes can also comprise or be accompanied with one or more other ingredients that facilitate the engraftment or functional mobilization of the cardiomyocytes. Suitable ingredients include matrix proteins that support or promote adhesion of the cardiomyocytes, or complementary cell types. In another embodiment, the composition may comprise resorbable or biodegradable matrix scaffolds.

Gene therapy can be used to either modify a cell to replace a gene product, to facilitate regeneration of tissue, to treat disease, or to improve survival of the cells following implantation into a subject (i.e., prevent rejection).

In one aspect of the present invention, a population of cardiomyocytes as disclosed herein is suitable for administering systemically or to a target anatomical site. A population of cardiomyocytes can be grafted into or nearby a subject's heart, for example, or may be administered systemically, such as, but not limited to, intra-arterial or intravenous administration. In alternative embodiments, a population of cardiomyocytes of the present invention can be administered in various ways as would be appropriate to implant in the cardiac system, including but not limited to parenteral, including intravenous and intraarterial administration, intrathecal administration, intraventricular administration, intraparenchymal, intracranial, intracisternal, intrastriatal, and intranigral administration. Optionally, a population of cardiomyocytes is administered in conjunction with an immunosuppressive agent.

In some embodiments, a population of cardiomyocytes can be administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement, including but not limited to improved survival rate or more rapid recovery, or improvement or elimination of symptoms and other indicators as are selected as appropriate measures by those skilled in the art. A population of cardiomyocytes can be administered to a subject at the following locations: clinic, clinical office, emergency department, hospital ward, intensive care unit, operating room, catheterization suites, and radiologic suites.

In other embodiments, a population of cardiomyocytes is stored for later implantation/infusion. A population of cardiomyocytes may be divided into more than one aliquot or unit such that part of a population of cardiomyocytes is retained for later application while part is applied immediately to the subject. Moderate to long-term storage of all or part of the cells in a cell bank is also within the scope of this invention, as disclosed in U.S. Patent Publication No. 2003/0054331 and Patent Publication No. WO 03/024215, and are incorporated by reference in their entireties. At the end of processing, the concentrated cells may be loaded into a delivery device, such as a syringe, for placement into the recipient by any means known to one of ordinary skill in the art.

In some embodiments a population of cardiomyocytes can be applied alone or in combination with other cells, tissue, tissue fragments, growth factors such as VEGF and other known angiogenic or arteriogenic growth factors, biologically active or inert compounds, resorbable plastic scaffolds, or other additives intended to enhance the delivery, efficacy, tolerability, or function of the population. In some embodiments, a population of cardiomyocytes may also be modified by insertion of DNA or by placement in cell culture in such a way as to change, enhance, or supplement the function of the cells for derivation of a structural or therapeutic purpose. For example, gene transfer techniques for stem cells are known by persons of ordinary skill in the art, as disclosed in (Morizono et al., 2003; Mosca et al., 2000), and may include viral transfection techniques, and more specifically, adeno-associated virus gene transfer techniques, as disclosed in (Walther and Stein, 2000) and (Athanasopoulos et al., 2000). Non-viral based techniques may also be performed as disclosed in (Murarnatsu et al., 1998).

In another aspect, in some embodiments, a population of cardiomyocytes could be combined with a gene encoding pro-angiogenic growth factor(s). Genes encoding anti-apoptotic factors or agents could also be applied. Addition of the gene (or combination of genes) could be by any technology known in the art including but not limited to adenoviral transduction, “gene guns,” liposome-mediated transduction, and retrovirus or lentivirus-mediated transduction, plasmid adeno-associated virus. Cells could be implanted along with a carrier material bearing gene delivery vehicle capable of releasing and/or presenting genes to the cells over time such that transduction can continue or be initiated. Particularly when the cells and/or tissue containing the cells are administered to a patient other than the patient from whom the cells and/or tissue were obtained, one or more immunosuppressive agents may be administered to the patient receiving the cells and/or tissue to reduce, and preferably prevent, rejection of the transplant. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell costimulation pathways, such as agents that interfere with the coupling of T-cells and B-cells via the CTLA4 and B7 pathways, as disclosed in U.S. Patent Publication No 2002/0182211, which is incorporated herein by reference. In one embodiment, an immunosuppressive agent is cyclosporine A. Other examples include myophenylate mofetil, rapamicin, and anti-thymocyte globulin. In one embodiment, the immunosuppressive drug is administered with at least one other therapeutic agent. The immunosuppressive drug is administered in a formulation which is compatible with the route of administration and is administered to a subject at a dosage sufficient to achieve the desired therapeutic effect. In another embodiment, the immunosuppressive drug is administered transiently for a sufficient time to induce tolerance to the cardiomyocytes of the invention.

Pharmaceutical compositions comprising effective amounts of a population of cardiomyocytes are also contemplated by the present invention. These compositions comprise an effective number of cardiomyocytes, optionally, in combination with a pharmaceutically acceptable carrier, additive or excipient. In certain aspects of the present invention, a population of cardiomyocytes is administered to the subject in need of a transplant in sterile saline. In other aspects of the present invention, a population of cardiomyocytes is administered in Hanks Balanced Salt Solution (HBSS) or Isolyte S. pH 7.4. Other approaches may also be used, including the use of serum free cellular media. In one embodiment, a population of cardiomyocytes is administered in plasma or fetal bovine serum, and DMSO. Systemic administration of a population of cardiomyocytes to the subject may be preferred in certain indications, whereas direct administration at the site of or in proximity to the diseased and/or damaged tissue may be preferred in other indications.

In some embodiments, a population of cardiomyocytes can optionally be packaged in a suitable container with written instructions for a desired purpose, such as the reconstitution or thawing (if frozen) of a population of cardiomyocytes prior to administration to a subject.

Methods of Identifying Cardiomyocyte Maturation Factors that Increase the Production of Mature Cardiomyocytes

Described herein is a method of identifying a cardiomyocyte maturation factor or agent that increases the production of cardiomyocytes (e.g., mature cardiomyocytes). In certain examples, a high content and/or high throughput screening method is provided. The method includes exposing at least one immature cardiomyocyte or a precursor thereof to at least one compound (e.g., a library compound or a compound described herein) and determining if the compound increases the production of cardiomyocytes, e.g., mature cardiomyocytes from the at least one immature cardiomyocyte or the precursor thereof. A cell can be identified as a cardiomyocyte (e.g., a mature cardiomyocyte) using one or more of the markers described herein. In some examples, the at least one immature cardiomyocyte or the precursor thereof may be differentiated prior to exposure to the library. In other examples, two or more compounds may be used, either individually or together, in the screening assay. In additional examples, the at least one immature cardiomyocyte or the precursor thereof may be placed in a multi-well plate, and a library of compounds may be screened by placing the various members of the library in different wells of the multi-well plate. Such screening of libraries can rapidly identify compounds that are capable of generating cardiomyocytes, e.g., mature cardiomyocytes, from the at least one immature cardiomyocyte or precursor thereof.

In some embodiments, the method further comprises isolating a population of the cardiomyocytes, e.g., mature cardiomyocytes (e.g., wherein at least 5%, 10%, 15%, 20%, 25%, 30%, 35%, 50%, 75% or greater are of the subject cell type).

In some embodiments, the method further comprises implanting the cardiomyocytes produced by the methods as disclosed herein into a subject (e.g., a subject having chronic heart failure). In some embodiments, the cardiomyocyte is derived from a stem cell obtained from a subject. In some embodiments, the cardiomyocyte is derived from a stem cell from a donor different than the subject, e.g., a relative of the subject.

In one aspect, the invention features a cardiomyocyte, e.g., a mature cardiomyocyte, made by a method described herein. In another aspect, the invention features a composition comprising a cardiomyocyte made by a method described herein.

In another aspect, the invention features a kit comprising: immature cardiomyocytes or precursors thereof; at least one cardiomyocyte maturation factor described herein; and instructions for using the immature cardiomyocytes or precursors thereof and the at least one cardiomyocyte maturation factor to produce a cardiomyocyte (e.g., a mature cardiomyocyte). In some embodiments, the kit further comprises: a component for the detection of a marker for a mature cardiomyocyte, e.g., for a marker described herein, e.g., a reagent for the detection of a marker of cardiomyocyte maturity, e.g., an antibody against the marker; and a mature cardiomyocyte, e.g., for use as a control.

In one aspect, the invention features a method of facilitating differentiation of immature cardiomyocytes or precursors thereof to cardiomyocytes comprising providing at least one immature cardiomyocyte or precursor thereof, and providing at least one cardiomyocyte maturation factor (e.g., 1, 2, 3, 4, 5, 6, 7, 8, 9, 10 or more cardiomyocyte maturation factors described herein) to mature or differentiate the at least one immature cardiomyocyte or precursor thereof to a cardiomyocyte (e.g., a mature cardiomyocyte), upon exposure of the stem cell to the at least one maturation factor. In some embodiments, the at least one immature cardiomyocyte or precursor thereof is from a mammal. In some embodiments, the at least one immature cardiomyocyte or precursor thereof is from mouse or human. In some embodiments, the at least one immature cardiomyocyte or precursor thereof derived from culturing an embryonic stem cell (e.g., a mammalian embryonic stem cell such as a mouse or human embryonic stem cell). In some embodiments, the at least one immature cardiomyocyte or precursor thereof derived from culturing an induced pluripotent stem cell (e.g., a mammalian iPs cell such as a mouse or human iPs cell).

In some embodiments, a plurality of immature cardiomyocytes or precursors thereof are differentiated or matured into a plurality of mature cardiomyocytes, for example, by contacting the plurality of immature cardiomyocytes or precursors thereof with at least one, at least two, at least three, or more of the cardiomyocyte maturation factors as described herein.

In some embodiments, the plurality of immature cardiomyocytes or precursors thereof are exposed to the cardiomyocyte maturation factors, for about 1, 2, 4, 6, 8, 10, 12, 14, 16, or more days. In some embodiments, the plurality of immature cardiomyocyte or precursors thereof are exposed to the cardiomyocyte maturation factors at a concentration of about 25 nM, 50 nM, 100 nM, 150 nM, 200 nM, 250 nM, 400 nM, 500 nM, 600 nM, 700 nM, 800 nM, 1 μM, 2 μM, 3 μM, 4 μM, 5 μM or 10 μM. In some embodiments, the plurality of immature cardiomyocytes or precursors thereof are exposed to the cardiomyocyte maturation factors at a concentration of about 250 nM, 400 nM, 500 nM, 600 nM, 700 nM, or 800 nM. In some embodiments, greater than about 20%, 30%, 40%, 50%, 60%, 70%, 80%, or 90% of the immature cardiomyocytes or precursors thereof are differentiated or matured into the mature cardiomyocytes.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the disclosure. Further, all patents, patent applications, and publications identified are expressly incorporated herein by reference for the purpose of describing and disclosing, for example, the methodologies described in such publications that might be used in connection with the disclosure. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents

### EXAMPLES

### Example 1: Human Stem Cell-Derived Endothelial Cells Suppress Automaticity of Stem Cell-Derived Cardiomyocytes

### Background

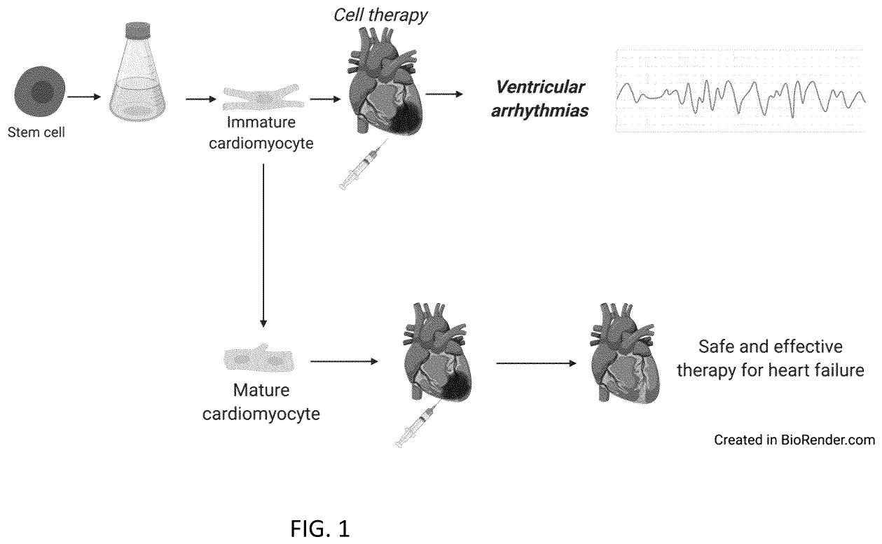

Inadequate maturation of cardiomyocytes has been identified as a barrier to clinical translation (FIG. 1). The delivery of immature stem cell-derived cardiomyocytes to large animal models may lead to potentially life-threatening ventricular arrhythmias. However, it has further been shown that after delivery of immature cardiomyocytes to animals, the cardiomyocytes undergo further maturation suggesting that other cells may interact with cardiomyocytes to promote their maturation. This led to the question of whether endothelial cells can facilitate cardiomyocyte maturation and survival.

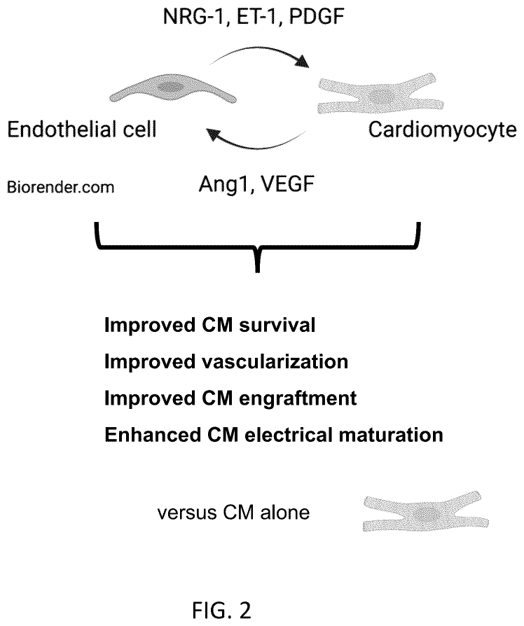

Endothelial cells are abundant in the heart making up about 60% of the non-cardiomyocyte cells in the heart. Endothelial cells (ECs) may promote maturation of cardiomyocytes through paracrine effects. It was hypothesized that the co-culture of iPSC-derived ECs with iPSC-derived CMs improves electrophysiological maturation of cardiomyocytes and reduces automaticity (FIG. 2).

### Methods and Materials

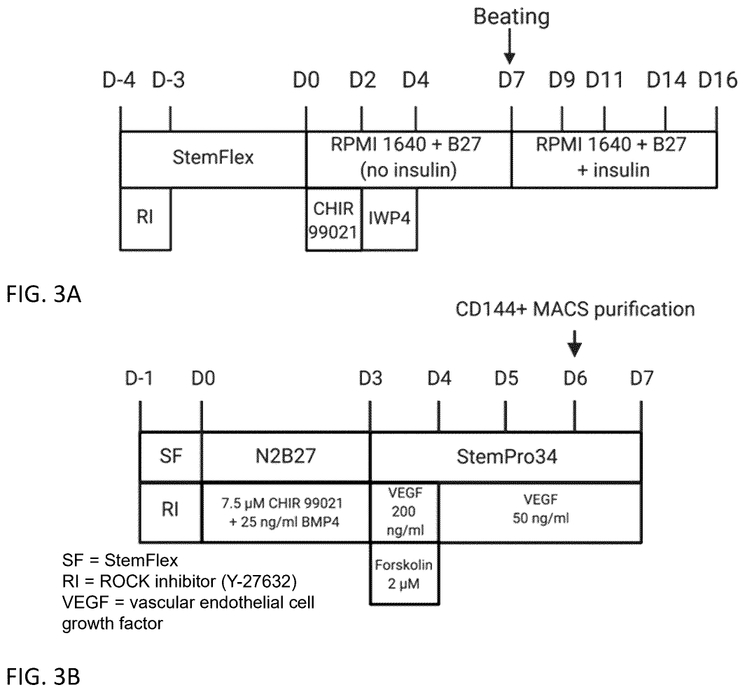

The cell lines used included UCSD142i-86-1 (WiCell) and Gibco episomal derived iPSCs (Thermo). Induced pluripotent stem cell-derived cardiomyocytes (iPSC-CMs) were obtained via a differentiation protocol adapted from Lian et al. Nat Protoc 2013; 8 (1): 162-75 (FIG. 3A). Induced pluripotent stem-cell derived endothelial cells (iPSC-ECs) were obtained via a differentiation protocol adapted from Patsch et al. Nat Cell Biol 2015; 17 (8): 994-1003 (FIG. 3B). iPSC-CMs were cultured with and without iPSC-ECs.

A mesh nanoelectronics device, such as that described in Li et al., Nano Lett 2019; 19:5781-5789, was used to evaluate electrophysiological properties of the cardiomyocytes over time. In addition, single cell RNAseq was performed to evaluate gene expression differences in cardiomyocytes (CMs) cultured with endothelial cells (ECs) versus CMs cultured alone.

### Results

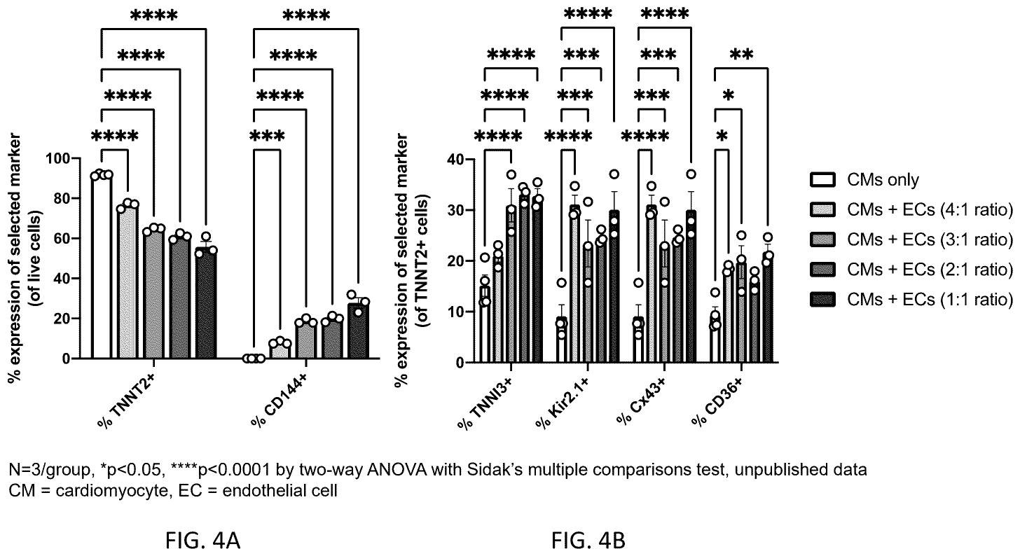

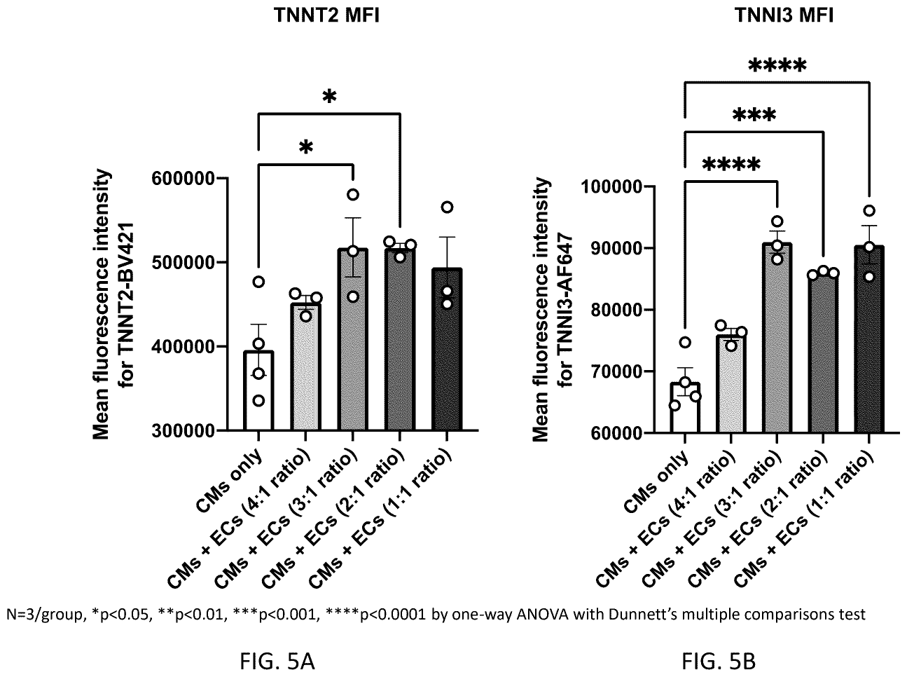

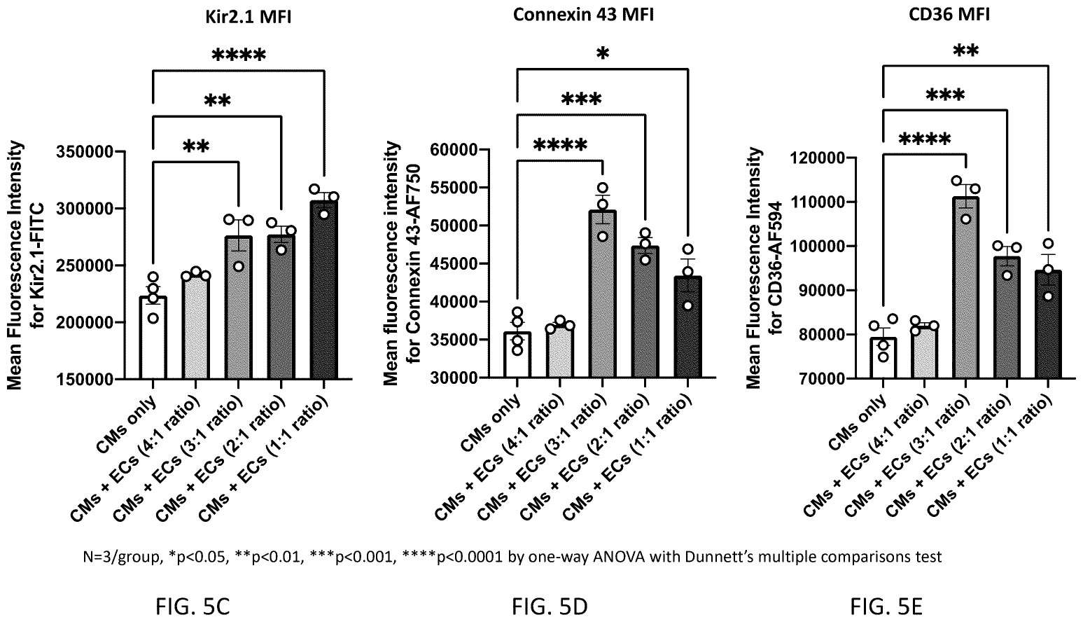

The co-culture of CMs with ECs resulted in increased expression of selected cardiomyocyte markers. For example, increased expression of TNNT2, TNNI3, Kir2.1, Cx43, and CD36 was shown (FIGS. 4A-4B). In some cases, the endothelial cell co-culture leads to increased expression of selected cardiac markers, such as Kir2.1 and Cx43, in a dose dependent manner by flow cytometry analysis (FIGS. 5A-5E).

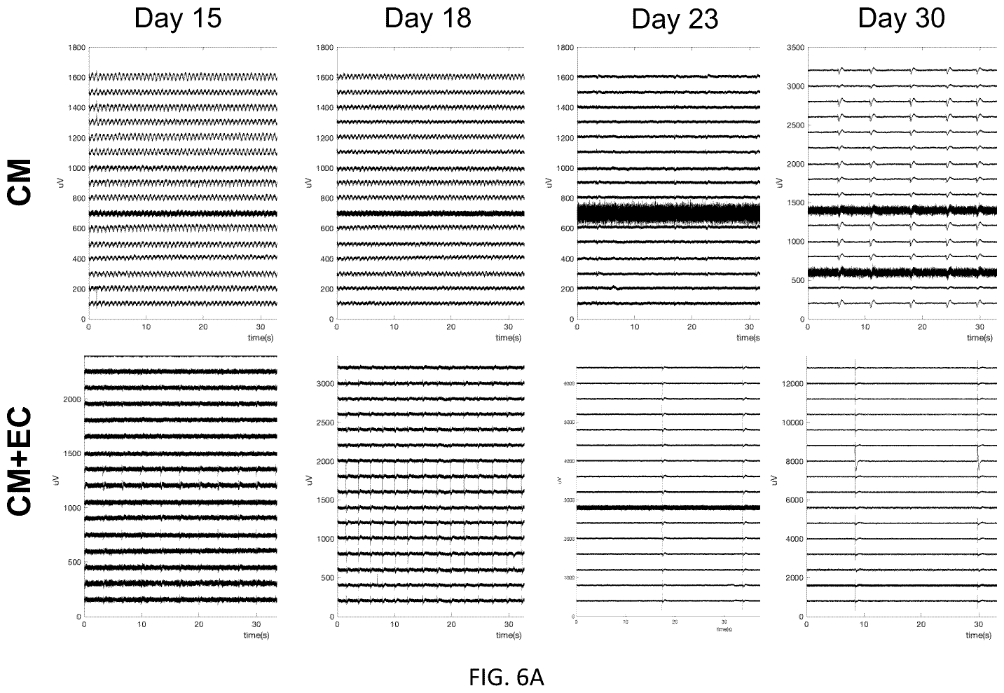

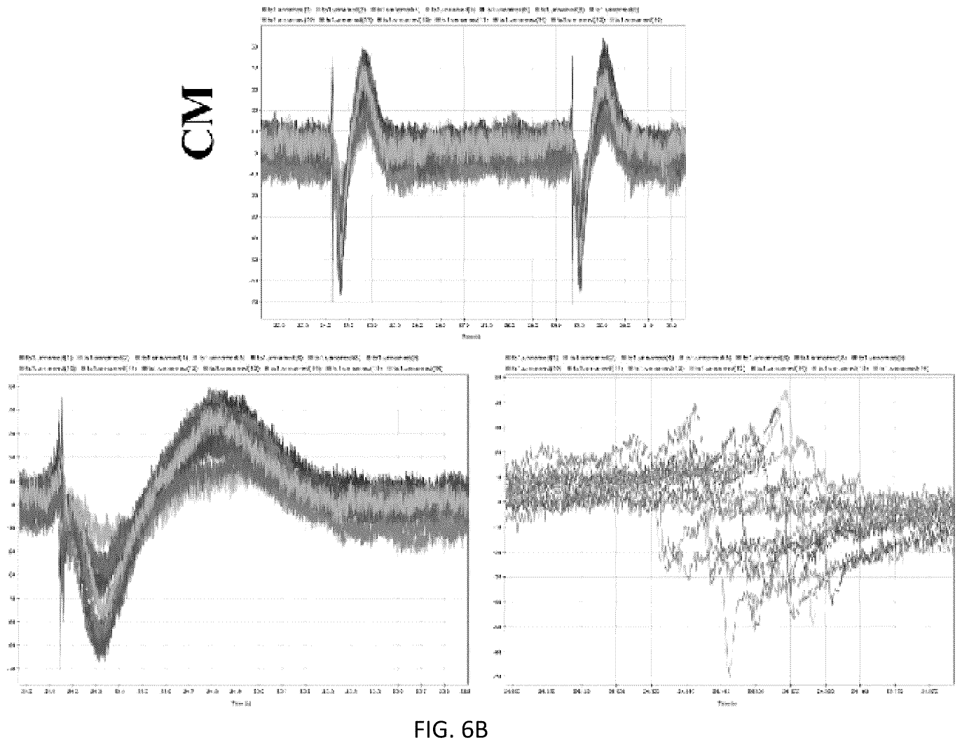

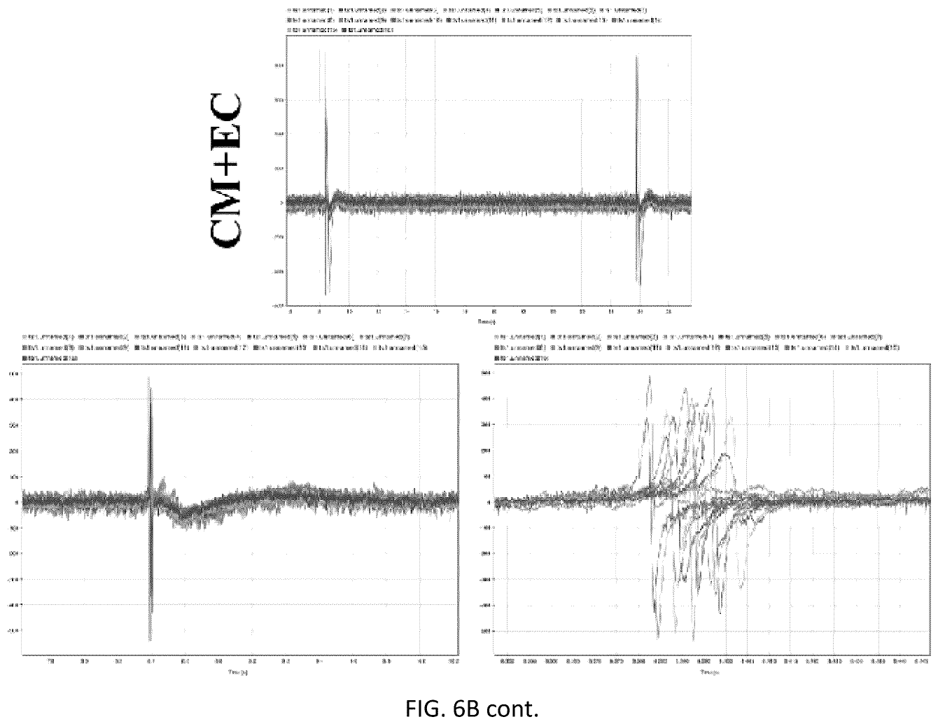

It was further found that iPSC-ECs may reduce the automaticity of iPSC-CMs. The CMs cultured on mesh nanoelectronics devices with ECs showed accelerated maturation of the electrical profile and a reduced beating rate (FIGS. 6A-6B).

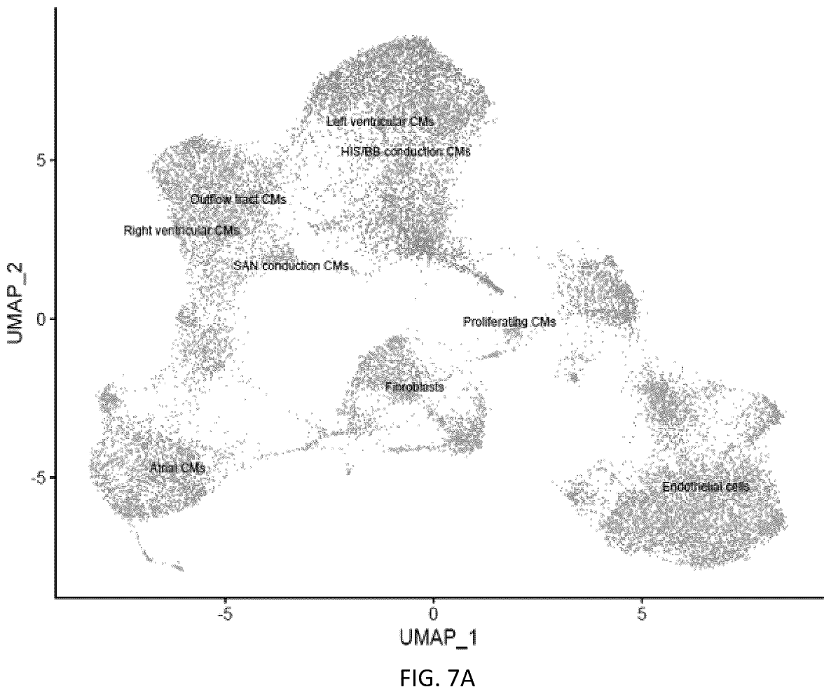

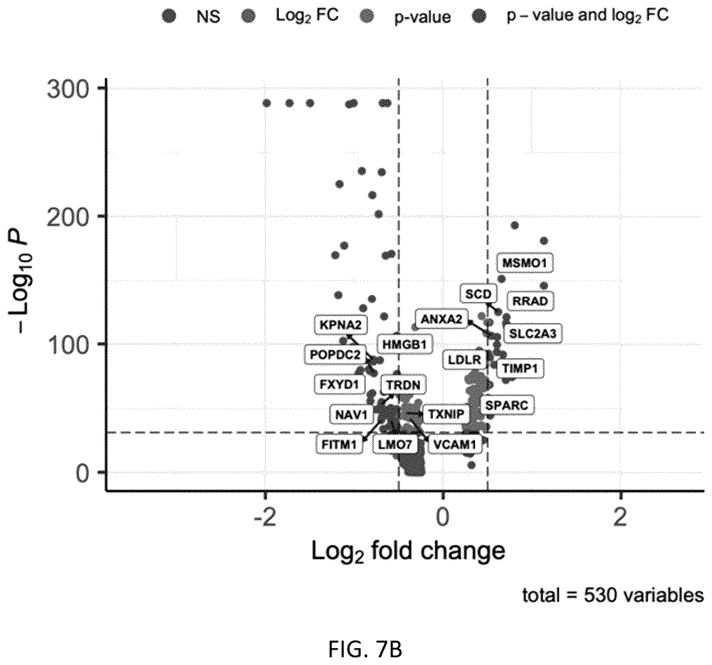

It was then questioned whether electrically immature cardiomyocytes could be isolated using surface markers. Single cell RNAseq was performed to reveal differences in gene expression in CMs co-cultured with ECs (CM+EC) versus CMs alone (FIG. 7A). The differential expression of potential cardiomyocyte surface markers was then examined for CM+EC vs. CM only (FIG. 7B). It is possible that one or more of these surface markers may be used to facilitate and drive maturation of the cardiomyocytes by overexpressing the protein.

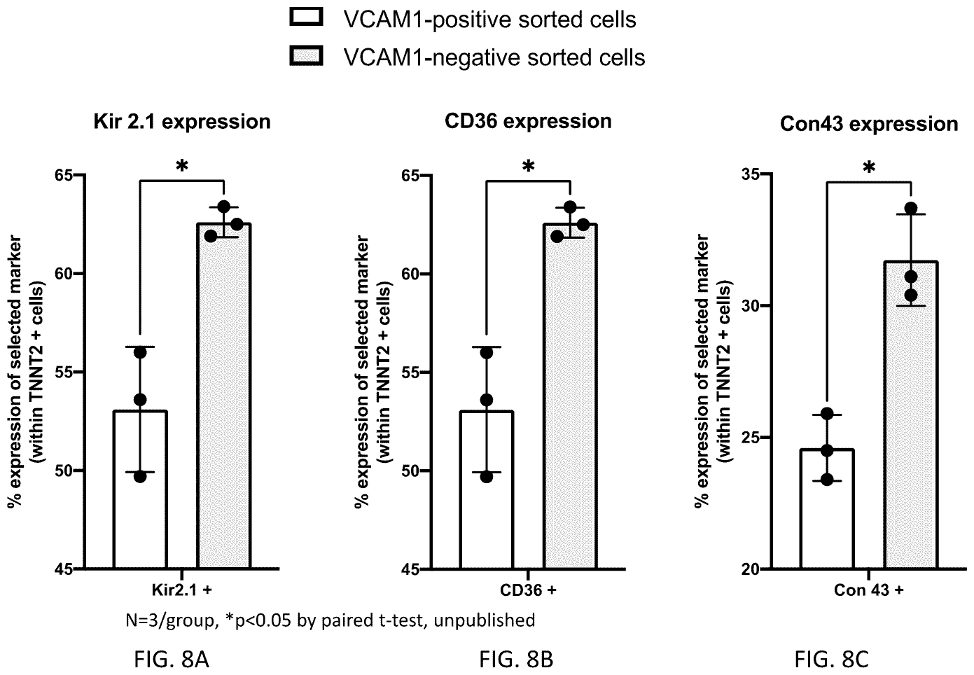

Finally, it was assessed whether VCAM1 can distinguish between cardiomyocytes of different maturity. Cardiomyocytes that were VCAM1-negative at the time of sorting had increased expression of Kir2.1, CD36, and connexin 43 (Cx43). Cardiomyocytes were sorted with the VCAM-1 marker and replated as positive VCAM-1 or negative VCAM1. After 5 days, they were analyzed with FACS (FIG. 8A-8C).

### Summary

The co-culture of cardiomyocytes (CMs) and endothelial cells (ECs) increases the expression of selected markers of cardiomyocyte maturation. The co-culture of CMs and ECs reduces the beating rate on a cyborg organoid and promotes a more mature electrical profile. Finally, work is ongoing to identify potential mechanisms to explain how ECs might enhance electrical maturation of CMs.

### Example 2: Human Endothelial Cells Mature Human Stem Cell-Derived Cardiomyocytes

### Background

Stem cell approaches to treat chronic heart failure will require production of ventricular cardiomyocytes to improve systolic heart function and reduce the incidence of ventricular arrhythmias. However, cardiomyocytes derived from embryonic or induced pluripotent stem cells (ESCs or iPSCs, respectively) using current differentiation protocols remain functionally immature. These immature cardiomyocytes display automaticity or pacemaker-like activity which results in potentially life-threatening ventricular arrhythmias when delivered to adult animal models and also have a less organized sarcomere structure preventing adequate contractile force (1, 2). Successful translation of stem cell-derived therapies for treatment of cardiovascular disease will require developing improved methods for maturation of stem cell-derived cardiomyocytes.

The heart contains other cell types that may potentially support the function and phenotype of cardiomyocytes. For example, macrophages can affect cardiomyocyte phenotype as reviewed previously (3). In addition, endothelial cells have been shown to enhance the maturation of cardiac progenitor cells (4). It has previously been shown that neonatal mouse cardiomyocytes have enhanced survival and maturation when co-cultured with adult mouse endothelial cells (5). There are currently no known publications using human iPS-derived endothelial cells specifically for the application of suppressing automaticity of cardiomyocytes derived from human stem cells in order to overcome the barrier of arrhythmia risk needed for clinical translation.

Another major barrier to clinical translation is limited survival and engraftment of injected cardiomyocytes, with only ˜10-30% of delivered cells remaining at the injection site within a few days after injection, and decreasing even further at later time points (6). Functional improvement following delivery of stem cell-derived cardiomyocytes will require adequate retention, survival, and vascularization of delivered cardiomyocytes. Several strategies have been proposed to improve engraftment of stem cell-derived cardiomyocytes (6). Heat shocking or ischemic pre-conditioning of cells prior to delivery induces expression of proteins that help cell survival, such as heat shock proteins or hypoxia inducible factor-1 (6). Use of a pro-survival cocktail which includes insulin-like growth factor-1 (IGF-1), cyclosporine (to inhibit opening of the mitochondrial membrane permeability transition pore), pinacidil (to open mitochondrial ATP-sensitive potassium channels to preserve the mitochondrial inner membrane potential), a Bcl-X_{L}-derived peptide (anti-apoptotic) and a caspase inhibitor improves engraftment and survival of cardiomyocytes at 4 weeks in a rat model of myocardial infarction (7). It will also be important to have adequate vascularization in the region of the delivered cardiomyocytes, as much higher engraftment rates and graft sizes are seen when cardiomyocytes are delivered to uninjured myocardium compared to infarcted myocardium (7, 8). Strategies to pre-vascularize infarcted tissue prior to cardiomyocyte delivery or techniques that support rapid assembly of a vascular network with cardiomyocyte delivery will likely improve engraftment and better support functional recovery following ischemic injury.

### Summary

Preliminary data is provided that shows that co-culture of human iPSC-ECs improves electrical maturation of human iPSC-CMs compared to iPSC-CMs alone. In addition, preliminary data showed survival of human iPSC-ECs following intramyocardial injection into athymic rat hearts with subsequent formation into capillary-like structures by 7 days.

These results suggest that vascular network formation may be possible from human iPSC-ECs that may promote survival and engraftment of iPSC-CMs in addition to promoting electrical maturation.

### Methods

The UCSD142i-86-1 cell line was used to generate preliminary data. UCSD142i-86-1 cells were differentiated into cardiomyocytes (9) or endothelial cells (10) according to previously published protocols adapted to three-dimensional culture in our laboratory. Endothelial cells were sorted by CD144 expression using magnetic-activated cell sorting. Initial experiments involved seeding iPSC-ECs and/or iPSC-CMs onto 2D culture plates coated with a peptide hydrogel. Immunostaining was performed to evaluate for vascular structure formation and cardiomyocyte survival and maturation. Western analysis was performed to evaluate cardiac troponin T and I expression. A live/dead assay kit was used to quantify cell survival. For electrical recordings, five million cells (5 million iPSC-CMs (on day 11 of differentiation) in cardiomyocyte-only group versus 1.3 million iPSC-ECs (day 7 of differentiation)+3.7 million iPSC-CMs (day 11 of differentiation) in co-culture group) were seeded onto nanomesh electrode devices developed by the Liu lab (11). Electrical recordings from cells in culture were taken at 15, 18, 23, and 30 days after initiation of cardiomyocyte differentiation.

In a separate experiment iPSC-ECs were delivered via echo-guided injection in the left ventricular free wall of athymic rats. Cyclosporine was used for immunosuppression beginning 1 day prior to cell injection and continuing for 7 days after injection. Hearts were harvested and prepared for immunostaining.

### Preliminary Data

### Endothelial Cell-Cardiomyocyte Interactions Enhance Cardiomyocyte Survival and Cardiac Troponin Expression In Vitro

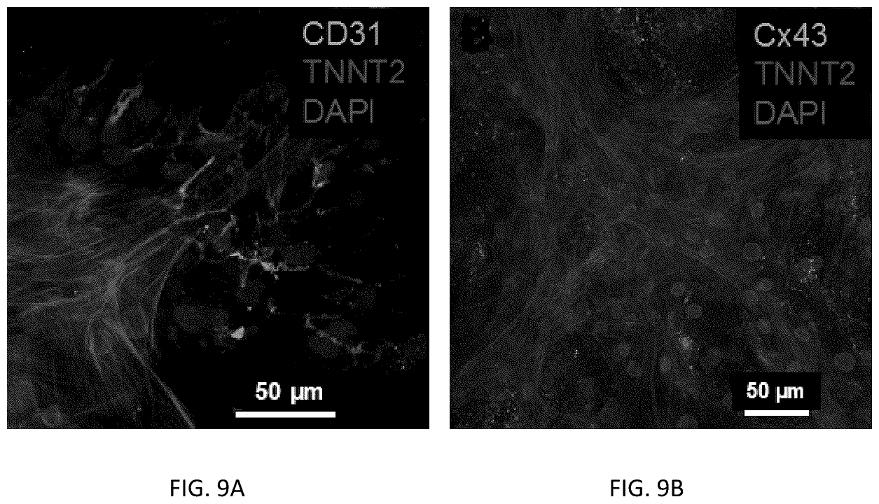

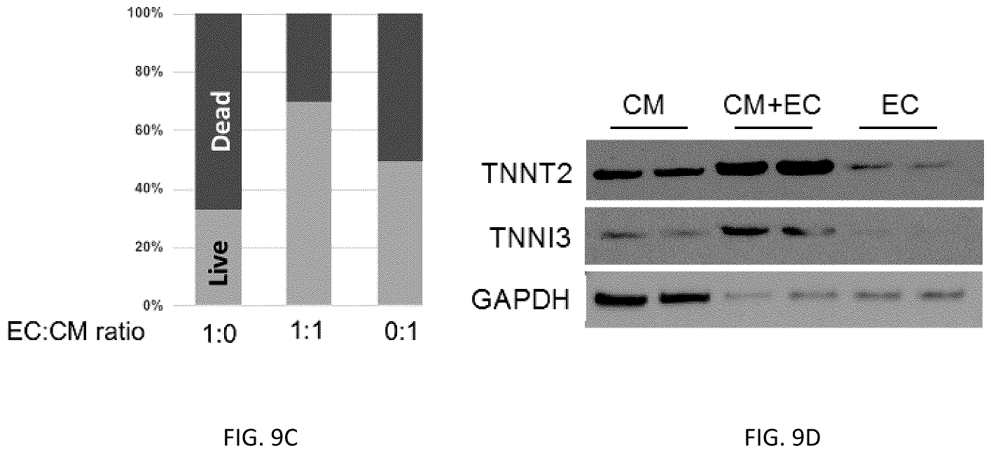

It was previously demonstrated that neonatal mouse cardiomyocytes have improved spatial organization, survival and electrical integration when co-cultured with adult mouse endothelial cells (5). The new preliminary data examined how the interactions between iPSC-derived cardiomyocytes (iPSC-CMs) and human iPSC-ECs support the survival, maturation, and electrical integration of human iPSC-CMs in vitro and in vivo. In preliminary data, iPSC-ECs provided structural organization for iPSC-CMs seeded in co-culture in SAPs (FIG. 9A), with Cx43 observed at the interface between the two cell types (FIG. 9B). Combining iPSC-ECs and iPSC-CMs increased the percentage of live cells compared to either alone (FIG. 9C). In addition, co-culture of iPSC-ECs with iPSC-CMs may increase expression of cardiac troponin T and cardiac troponin I compared to iPSC-CMs alone (FIG. 9D), suggesting that the presence of iPSC-ECs may facilitate aspects of iPSC-CM maturation.

A stretchable mesh nanoelectronics device has been developed that can perform electrical recordings at different time points to evaluate the evolution of the electrical profile of cardiomyocytes over time. This device contains 64 electrodes capable of detecting the electrical signal from different locations after seeding of iPSC-CMs on the device. Electrical recordings can be measured from the device multiple times, allowing one to track how cells mature over time. iPSC-CMs alone exhibited a gradual transition over time with rapid, wide electrical profiles for each beat on day 15 of differentiation that transitions to a slower but still wide electrical profile by day 30 (FIG. 6A). In contrast, when iPSC-ECs were co-cultured by mixing with iPSC-CMs, the iPSC-CMs had accelerated maturation. On day 15 of differentiation, co-cultured iPSC-CMs already exhibited evidence of narrow electrical spikes suggestive of rapid depolarization consistent with that expected from a mature, adult cardiomyocyte. This narrow electrical pattern persisted through day 30 with all channels showing coordinated electrical propagation with a rapid depolarization phase (FIG. 6B). In addition, the rate of spontaneous beating was much slower in iPSC-CMs co-cultured with iPSC-ECs at each time point compared to iPSC-CMs alone, with a spontaneous beating rate <3 beats per minute in co-cultured iPSC-CMs on day 30 of differentiation. This slow spontaneous beating rate is characteristic of mature ventricular cardiomyocytes. It would be expected that this reduction in automaticity, or propensity for spontaneous beating, exhibited by iPSC-CMs co-cultured with iPSC-ECs, would significantly reduce the risk of arrhythmias after delivery in vivo.

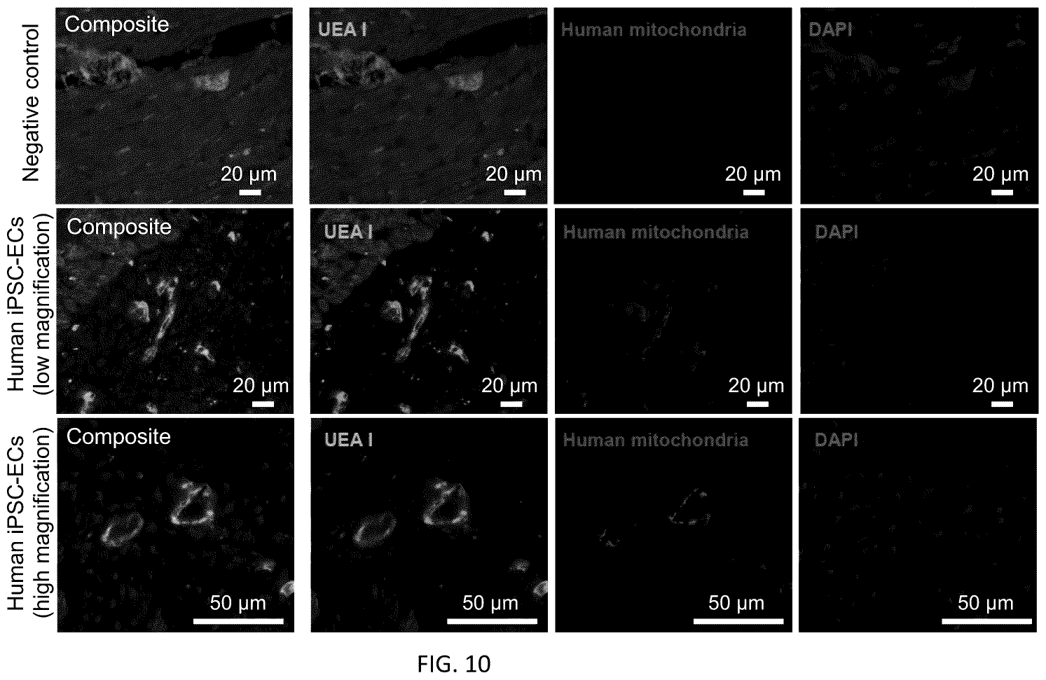

human iPSC-ECs were injected into athymic rat myocardium and then tissues were harvested at 7 days after injection (FIG. 10). Cyclosporine was used for immunosuppression. Vessel like structures were formed that stain positive for EC marker, CD31, and human-specific EC marker, Ulex Europaus agglutinin (UEA I), suggesting that iPSC-ECs formed microvessels within the rat myocardium within 1 week after delivery.

### Summary

In summary, human endothelial cells may be required for electrical maturation of human stem cell-derived cardiomyocytes. The results suggest that iPSC-ECs improve iPSC-CM survival and electrical maturation in vivo and can form vascular structures in vivo that may also facilitate iPSC-CM survival after delivery. If successful, the ability of iPSC-CMs to suppress automaticity of iPSC-CMs and improve survival of iPSC-CMs can help overcome two major hurdles (arrhythmia risk, engraftment) to clinical translation of cardiomyocyte cell therapies.

### REFERENCES

1. Chong J J, Yang X. Don C W, Minami E, Liu Y W, Weyers J J, Mahoney W M, Van Biber B, Cook S M, Palpant N J, Gantz J A, Fugate J A, Muskheli V, Gough G M, Vogel K W, Astley C A, Hotchkiss C E, Baldessari A, Pabon L, Reinecke H, Gill E A, Nelson V, Kiem H P, Laflamme M A, Murry C E. Human embryonic-stem-cell-derived cardiomyocytes regenerate non-human primate hearts. Nature. 2014; 510 (7504): 273-7. doi: 10.1038/nature13233. PubMed PMID: 24776797; PMCID: PMC4154594.
2. Yang X, Pabon L, Murry C E. Engineering adolescence: maturation of human pluripotent stem cell-derived cardiomyocytes. Circ Res. 2014; 114 (3): 511-23. doi: 10.1161/CIRCRESAHA.114.300558. PubMed PMID: 24481842; PMCID: PMC3955370.
3. Sansonetti M, Waleczek F J G, Jung M, Thum T, Perbellini F. Resident cardiac macrophages: crucial modulators of cardiac (patho) physiology. Basic Res Cardiol. 2020; 115 (6): 77. Epub 2020 Dec. 8. doi: 10.1007/s00395-020-00836-6. PubMed PMID: 33284387; PMCID: PMC7720787.
4. Dunn K K, Reichardt I M, Simmons A D, Jin G, Floy M E, Hoon K M, Palecek S P. Coculture of Endothelial Cells with Human Pluripotent Stem Cell-Derived Cardiac Progenitors Reveals a Differentiation Stage-Specific Enhancement of Cardiomyocyte Maturation. Biotechnol J. 2019; 14 (8): c1800725. Epub 2019 Mar. 31. doi: 10.1002/biot.201800725. PubMed PMID: 30927511; PMCID: PMC6849481.
5. Narmoneva D A, Vukmirovic R, Davis M E, Kamm R D, Lee R T. Endothelial cells promote cardiac myocyte survival and spatial reorganization: implications for cardiac regeneration. Circulation. 2004; 110 (8): 962-8. Epub 2004 Aug. 11. doi: 10.1161/01.CIR.0000140667.37070.07. PubMed PMID: 15302801; PMCID: PMC2754572.
6. Don C W, Murry C E. Improving survival and efficacy of pluripotent stem cell-derived cardiac grafts. J Cell Mol Med. 2013; 17 (11): 1355-62. Epub 2013 Oct. 15. doi: 10.1111/jcmm.12147. PubMed PMID: 24118766; PMCID: PMC4049630.
7. Laflamme M A, Chen K Y, Naumova A V, Muskheli V, Fugate J A, Dupras S K, Reinecke H, Xu C, Hassanipour M, Police S, O'Sullivan C, Collins L, Chen Y, Minami E, Gill E A, Ueno S, Yuan C, Gold J, Murry C E. Cardiomyocytes derived from human embryonic stem cells in pro-survival factors enhance function of infarcted rat hearts. Nat Biotechnol. 2007; 25 (9): 1015-24. Epub 2007 Aug. 28. doi: 10.1038/nbt1327. PubMed PMID: 17721512.
8. Zhang M, Methot D. Poppa V, Fujio Y, Walsh K. Murry C E. Cardiomyocyte grafting for cardiac repair: graft cell death and anti-death strategies. J Mol Cell Cardiol. 2001; 33 (5): 907-21. Epub 2001 May 10. doi: 10.1006/jmcc.2001.1367. PubMed PMID: 11343414.
9. Lian X, Zhang J, Azarin S M, Zhu K, Hazeltine L B, Bao X, Hsiao C. Kamp T J, Palecek S P. Directed cardiomyocyte differentiation from human pluripotent stem cells by modulating Wnt/beta-catenin signaling under fully defined conditions. Nat Protoc. 2013; 8 (1): 162-75. Epub 2012 Dec. 22. doi: 10.1038/nprot.2012.150. PubMed PMID: 23257984; PMCID: PMC3612968.
10. Patsch C, Challet-Meylan L, Thoma E C, Urich E, Heckel T, O'Sullivan J F, Grainger S J, Kapp F G, Sun L, Christensen K, Xia Y, Florido M H, He W, Pan W, Prummer M, Warren C R, Jakob-Roetne R, Certa U, Jagasia R. Freskgard P O, Adatto I, Kling D, Huang P, Zon L I, Chaikof E L, Gerszten R E, Graf M, lacone R, Cowan C A. Generation of vascular endothelial and smooth muscle cells from human pluripotent stem cells. Nat Cell Biol. 2015; 17 (8): 994-1003. Epub 2015 Jul. 28. doi: 10.1038/ncb3205. PubMed PMID: 26214132; PMCID: PMC4566857.
11. Li Q, Nan K, Le Floch P. Lin Z, Sheng H, Blum T S, Liu J. Cyborg Organoids: Implantation of Nanoelectronics via Organogenesis for Tissue-Wide Electrophysiology. Nano Lett. 2019; 19 (8): 5781-9. Epub 2019 Jul. 28. doi: 10.1021/acs.nanolett.9b02512. PubMed PMID: 31347851.

### Example 3: Development of Electrically-Mature Pluripotent Stem Cell-Derived Cardiomyocytes in Three-Dimensional Culture

### Project Summary

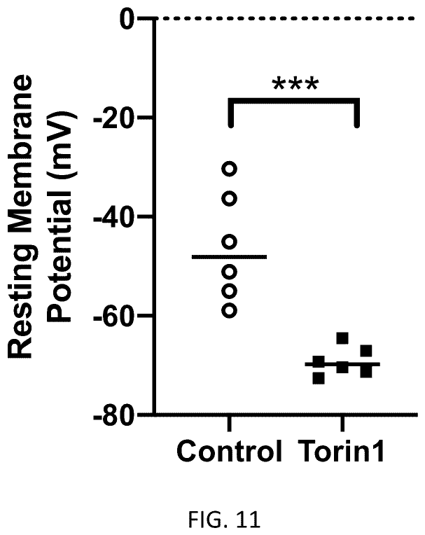

Pluripotent stem cell-derived cardiomyocytes (PSC-CMs) offer a regenerative approach to treat patients with systolic heart failure. However, delivery of immature PSC-CMs may increase the risk of ventricular arrhythmias, which is a barrier to clinical translation (1, 2). It was recently identified that inhibition of the mechanistic target of rapamycin (mTOR) pathway improves maturation of cardiomyocytes derived from induced pluripotent stem cells (iPSCs) in two-dimensional (2D) culture (3). The preliminary data suggest that mTOR inhibition can decrease the resting membrane potential of iPSC-derived cardiomyocytes in 2D in just 2 weeks, including differentiation time (FIG. 11). This could greatly simplify the manufacturing process compared to other protocols that take 2 months to produce mature PSC-CMs with similar resting membrane potentials (4).

To develop replacement therapy using allogeneic human PSC-CMs as a viable therapeutic option, methods to culture large numbers of PSC-CMs with good manufacturing practices for off-the-shelf use must be developed. Maintenance of 2D cultures is labor-intensive with significant batch-to-batch variability (5); in contrast, maintenance and differentiation of PSCs in three-dimensional (3D) bioreactor systems is more amenable to scale up, reduce labor time, and small volume sampling allows for improved quality control (6). However, this shift from 2D to 3D culture alters the phenotype of the cells due to differential regulation of various signaling pathways in different culture geometries including mTOR (7). The field has yet to devise a uniform protocol that efficiently produces mature PSC-CMs in 3D. The preliminary data suggest that mTOR inhibition has minimal effect on maturation in 3D culture, but other small molecules may have a more pronounced effect on PSC-CM maturation in 3D culture. It was proposed that electrically-mature human pluripotent stem cell-derived cardiomyocytes (PSC-CMs) would be generated utilizing the following project goals:
1. To test whether p53 activation with nutlin-3a enhances electrical maturation of iPSC-derived cardiomyocytes in 3D culture;
2. To test whether senescence suppression with senolytics such as quercetin can enhance electrical maturation of iPSC-CMs in 3D culture;
3. To test whether inhibition of FOXM1 signaling and/or activation of FOXO signaling can enhance electrical maturation of iPSC-CMs in 3D culture in vitro; and
4. To test whether manipulation of nutritional substrates in combination with small molecules can produce a rapid 3D protocol for mature human cardiomyocyte generation.

### Preliminary Data

### Nutlin-3a but not Torin1 Increases Expression of Kir2.1 and p53 in 3D Culture

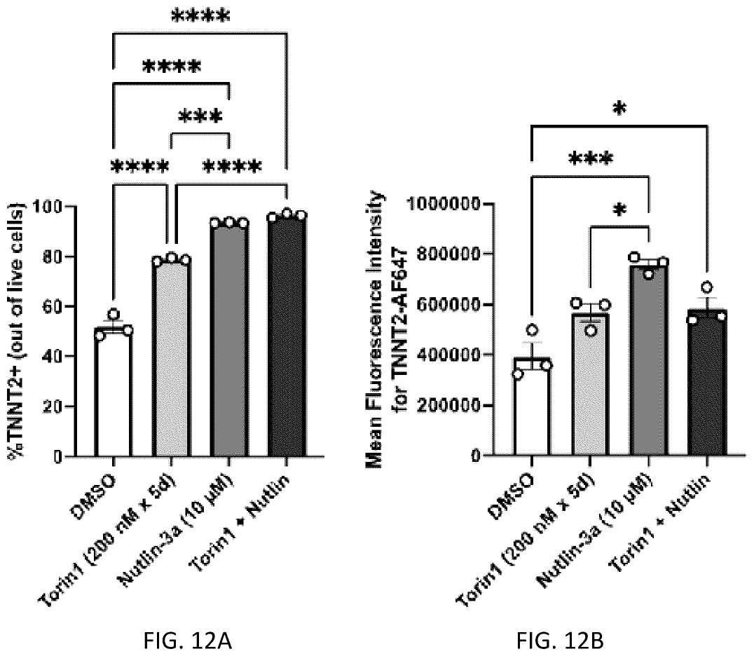

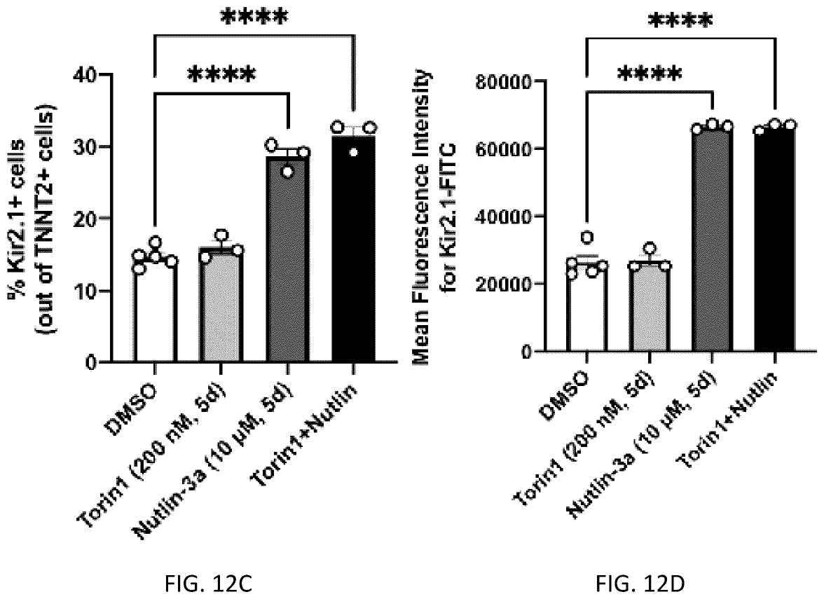

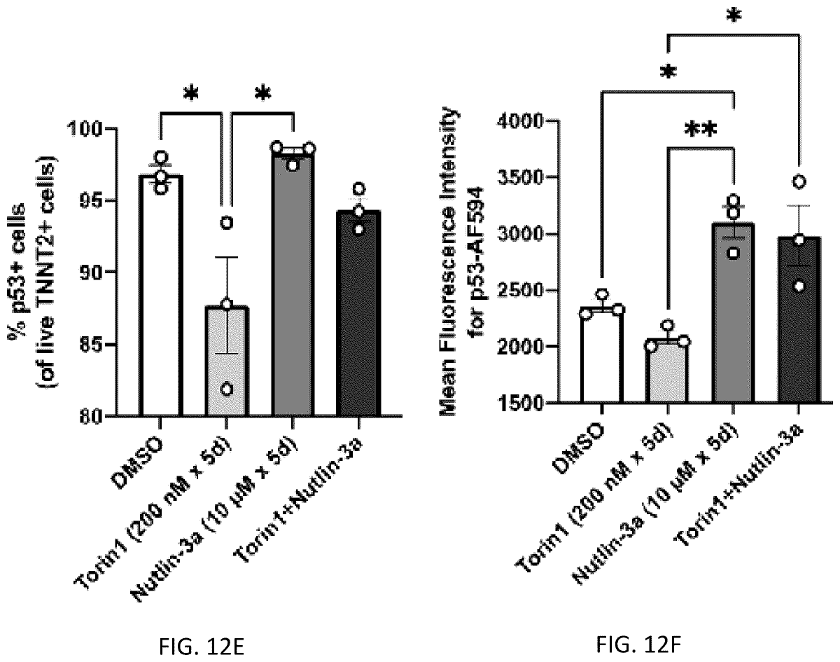

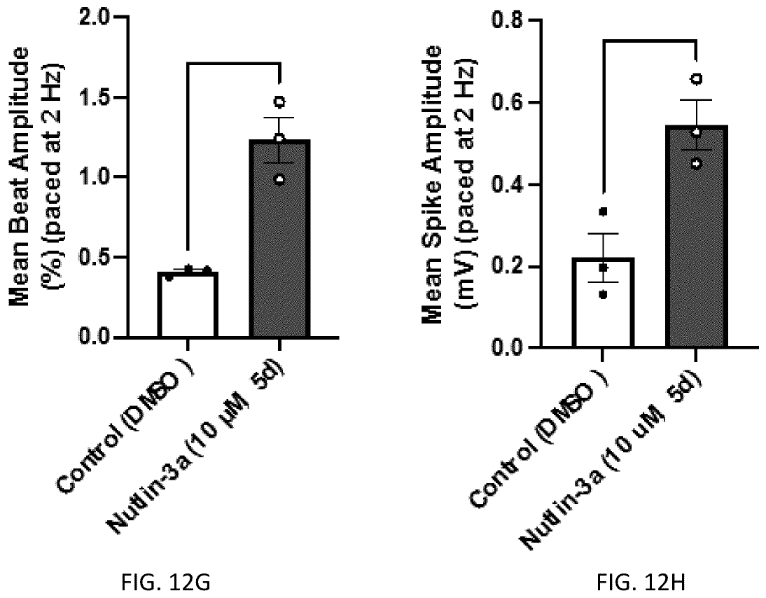

Kir2.1 is the ion channel largely responsible for maintaining the resting membrane potential via the inward rectifier current (I_{K1}); at lower levels, abnormal membrane depolarization can increase the risk of ventricular arrhythmias (8, 9). Nutlin-3a but not Torin1 treatment of iPSC-CMs increased expression of TNNT2 and Kir2.1 (FIG. 12A-12D). Also, Nutlin-3a but not Torin1 increased expression of p53, which may support induction of a quiescent state (FIG. 12E-12F). The preliminary data suggest that mTOR inhibition does not have a beneficial effect in 3D culture compared to 2D culture, possibly due to contact inhibition reducing mTOR activity (7). Using a multielectrode array (Axion Maestro Edge), Nutlin-3a increased the mean beat amplitude and mean spike amplitude, suggesting more mature contractile and electrophysiological phenotypes, respectively (FIG. 12G-12H).

Senescence Suppression with the Senolytic, Quercetin, Increases Expression of Kir2.1

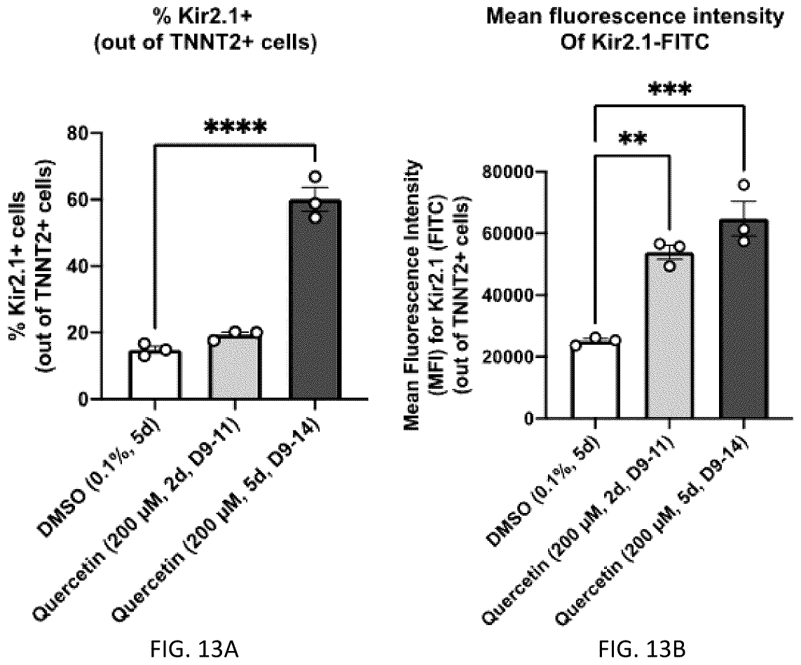

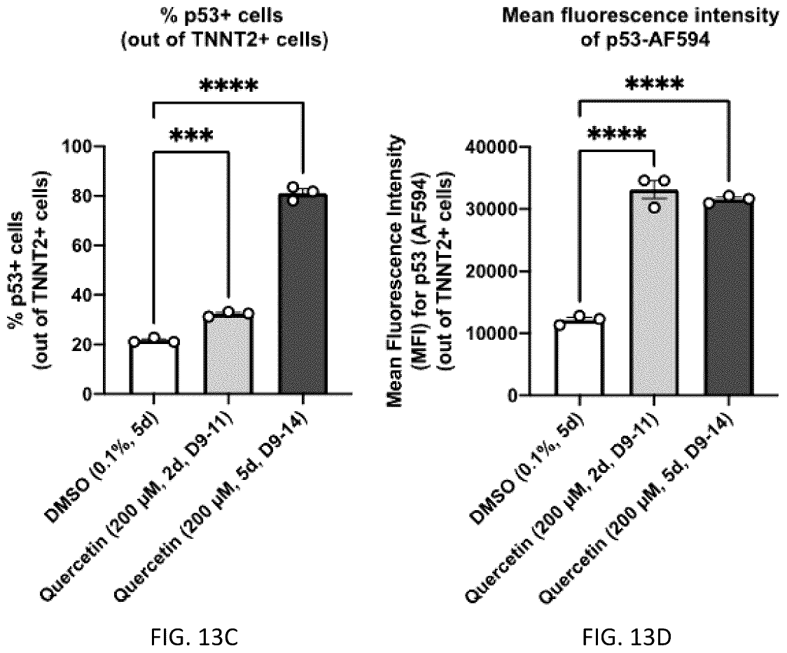

Quercetin treatment of iPSC-CMs increased expression of both Kir2.1 and p53 (FIG. 13), providing evidence that quercetin may facilitate electrical maturation and quiescence of iPSC-CMs.

Inhibition of FOXM1 with RCM1 Increases Expression of Kir2.1

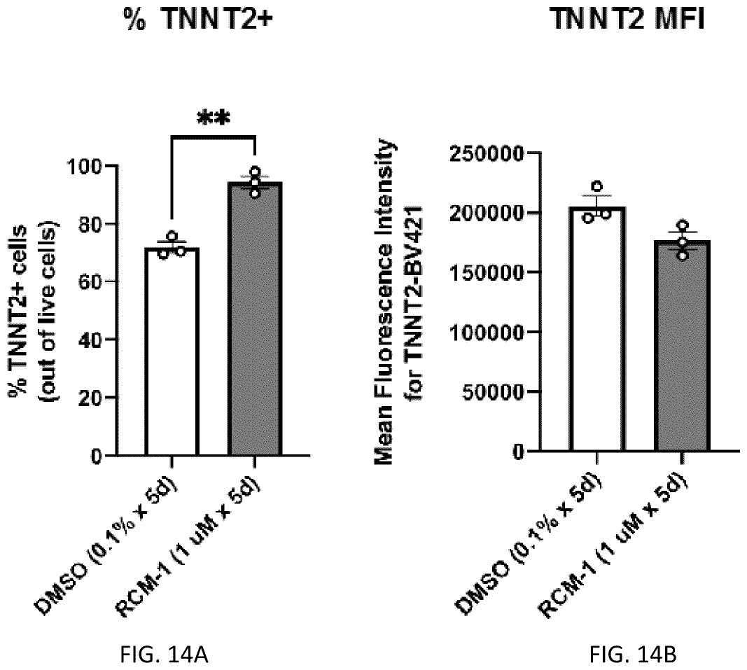

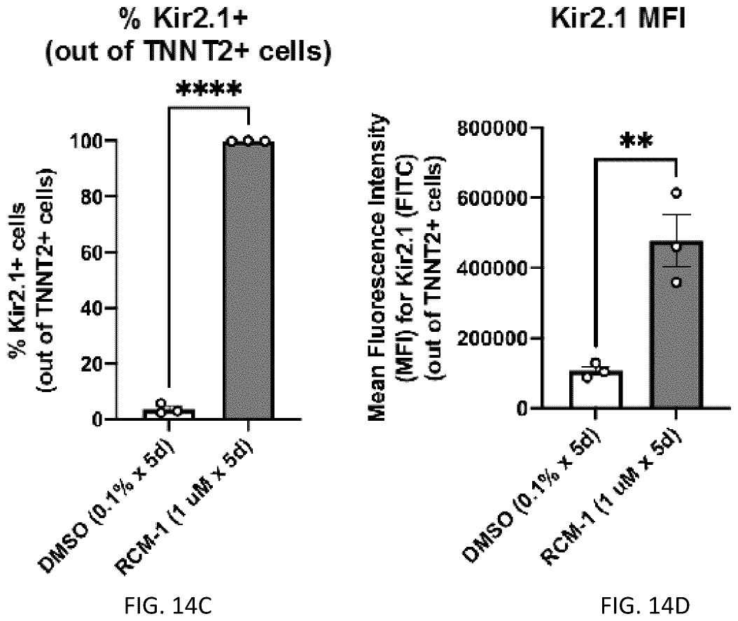

Inhibition of the forkhead box M1 (FOXM1) transcription factor with the small molecule, RCM1, increased purity of iPSC-CMs and increased expression of Kir2.1 in iPSC-CMs (FIG. 14), providing evidence that FOXM1 inhibition may facilitate electrical maturation of iPSC-CMs.

### Research Plan

It was proposed to develop a current Good Manufacturing Practice (cGMP)-complaint protocol capable of generating electrically-mature human PSC-CMs in 3D suspension culture using the Eppendorf DASbox Mini Bioreactor system currently used by BlueRock. Different small molecules will be tested that activate p53 (e.g. Nutlin-3a), senescence will be suppressed (using senolytics such as quercetin, dasatinib, or navitoclax), FOXM1 will be inhibited (e.g. RCM1) and/or FOXO will be activated (e.g. LOM612). For each of these pathways, a screen will be performed with different small molecule compounds at different concentrations, durations, and time points using expression of extracellular Kir2.1 quantified by flow cytometry as an initial screening endpoint. A follow-up will be performed via screening of calcium handling properties and action potential characteristics using a multi-electrode array system available. To obtain resting membrane potential and I_{K1} (inward rectifier potassium current), patch clamp work will be outsourced to a collaborator. A benchmark of RMP≤−70 mV will be used, which would demonstrate adequate improvement over current protocols to proceed with large animal studies to assess arrhythmia potential. Full phenotypic characterization will be performed on cells from the most promising treatment conditions, including evaluating RNA and protein expression of sarcomere, ion channel and metabolic genes, quantifying contractility, electrophysiological properties, and oxygen consumption rate.

### REFERENCES

1. Liu Y W, Chen B. Yang X, Fugate J A, Kalucki F A, Futakuchi-Tsuchida A, Couture L, Vogel K W, Astley C A, Baldessari A, Ogle J, Don C W, Steinberg Z L, Seslar S P, Tuck S A, Tsuchida H, Naumova A V, Dupras S K, Lyu M S, Lee J, Hailey D W, Reinecke H, Pabon L, Fryer B H, MacLellan W R. Thies R S, Murry C E. Human embryonic stem cell-derived cardiomyocytes restore function in infarcted hearts of non-human primates. Nat Biotechnol. 2018; 36 (7): 597-605. Epub 2018 Jul. 4. doi: 10.1038/nbt.4162. PubMed PMID: 29969440; PMCID: PMC6329375.
2. Romagnuolo R, Masoudpour H, Porta-Sanchez A, Qiang B, Barry J, Laskary A, Qi X, Masse S, Magtibay K, Kawajiri H, Wu J, Valdman Sadikov T, Rothberg J, Panchalingam K M, Titus E, Li R K, Zandstra P W, Wright G A, Nanthakumar K, Ghugre N R, Keller G, Laflamme M A. Human Embryonic Stem Cell-Derived Cardiomyocytes Regenerate the Infarcted Pig Heart but Induce Ventricular Tachyarrhythmias. Stem Cell Reports. 2019; 12 (5): 967-81. Epub 2019 May 6. doi: 10.1016/j.stemcr.2019.04.005. PubMed PMID: 31056479; PMCID: PMC6524945.
3. Garbern J C, Escalante G O, Lec R T. Pluripotent stem cell-derived cardiomyocytes for treatment of cardiomyopathie damage: Current concepts and future directions. Trends Cardiovasc Med. 2020. Epub 2020 Jan. 28. doi: 10.1016/j.tcm.2020.01.002. PubMed PMID: 31983535.
4. Feyen D A M, McKeithan W L, Bruyneel A A N, Spiering S, Hormann L, Ulmer B, Zhang H. Briganti F, Schweizer M, Hegyi B, Liao Z, Polonen R P, Ginsburg K S, Lam C K, Serrano R. Wahlquist C, Kreymerman A, Vu M, Amatya P L, Behrens C S, Ranjbarvaziri S. Maas R G C, Greenhaw M, Bernstein D, Wu J C, Bers D M, Eschenhagen T, Metallo C M, Mercola M. Metabolic Maturation Media Improve Physiological Function of Human iPSC-Derived Cardiomyocytes. Cell Rep. 2020; 32 (3): 107925. Epub 2020 Jul. 23. doi: 10.1016/j.celrep.2020.107925. PubMed PMID: 32697997.
5. Lian X, Zhang J, Azarin S M, Zhu K. Hazeltine L B, Bao X, Hsiao C, Kamp T J, Palecek S P. Directed cardiomyocyte differentiation from human pluripotent stem cells by modulating Wnt/beta-catenin signaling under fully defined conditions. Nat Protoc. 2013; 8 (1): 162-75. Epub 2012 Dec. 22. doi: 10.1038/nprot.2012.150. PubMed PMID: 23257984; PMCID: PMC3612968.
6. Kempf H, Kropp C. Olmer R. Martin U, Zweigerdt R. Cardiac differentiation of human pluripotent stem cells in scalable suspension culture. Nat Protoc. 2015; 10 (9): 1345-61. doi: 10.1038/nprot.2015.089. PubMed PMID: 26270394.
7. Riedl A, Schlederer M, Pudelko K, Stadler M, Walter S, Unterleuthner D, Unger C. Kramer N, Hengstschlager M, Kenner L. Pfeiffer D, Krupitza G, Dolznig H. Comparison of cancer cells in 2D vs 3D culture reveals differences in AKT-mTOR-S6K signaling and drug responses. J Cell Sci. 2017; 130 (1): 203-18. Epub 2016 Sep. 25. doi: 10.1242/jcs.188102. PubMed PMID: 27663511.
8. Jonsson M K, Vos M A, Mirams G R, Duker G, Sartipy P. de Boer T P, van Veen T A. Application of human stem cell-derived cardiomyocytes in safety pharmacology requires caution beyond hERG. J Mol Cell Cardiol. 2012; 52 (5): 998-1008. Epub 2012 Feb. 23. doi: 10.1016/j.yjmcc.2012.02.002. PubMed PMID: 22353256.
9. Goversen B, van der Heyden M A G, van Veen TAB, de Boer T P. The immature electrophysiological phenotype of iPSC-CMs still hampers in vitro drug screening: Special focus on IK1. Pharmacol Ther. 2018; 183:127-36. Epub 2017 Oct. 8. doi: 10.1016/j.pharmthera.2017.10.001. PubMed PMID: 28986101.

### Example 4: Dysregulation of FOXO-FOXM1 Signaling Inhibits Maturation of iPSC-Derived Cardiomyocytes in 3D Suspension Culture

### Background

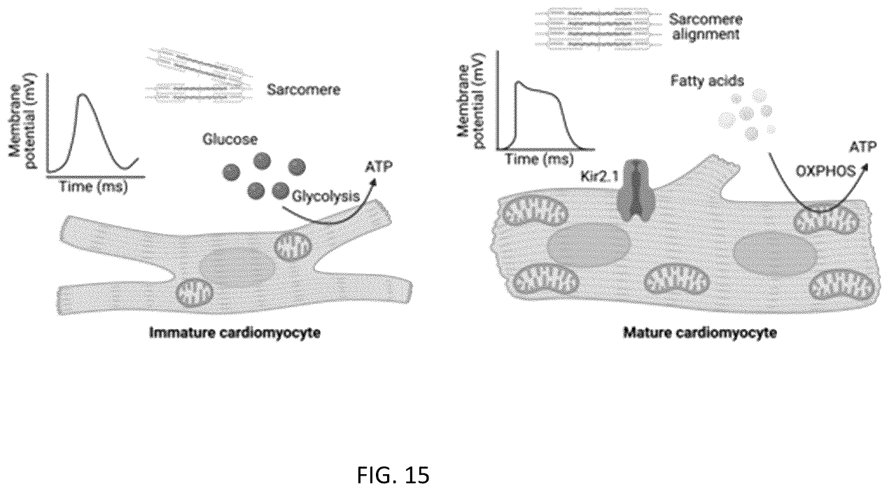

Inadequate maturation of stem cell-derived cardiomyocytes is a major barrier to clinical translation. Cardiomyocytes (CMs) derived from stem cells remain in an immature state more closely resembling fetal cardiomyocytes than mature, adult cardiomyocytes (FIG. 15). Immature stem cell-derived cardiomyocytes exhibit automaticity, or spontaneous beating, that increases the risk of ventricular arrhythmias after delivery to large animal models. Recent work in the field demonstrates that metabolic parameters regular cardiomyocyte maturation.

The forkhead box (FOX) family of transcription factors consists of about 50 proteins involved in fine tuning multiple biological processes. FOXO is involved in the regulation of cell cycle, reactive oxygen species, metabolism, and DNA repair and can inhibit FOXM1. FOXM promotes cell cycle activity. The proliferation or regulation of metabolic phenotypes and proliferation potential in neonatal cardiomyocytes is regulated via a balance between FOXO and FOXM activity. It is not yet understood whether FOXO-FOXM1 signaling participates in maturation of pluripotent stem cell (PSC)-derived cardiomyocytes. It was hypothesized that dysregulation of FOXO-FOXM1 signaling inhibits the maturation of iPSC-derived cardiomyocytes (iPSC-CMs).

### Methods and Materials

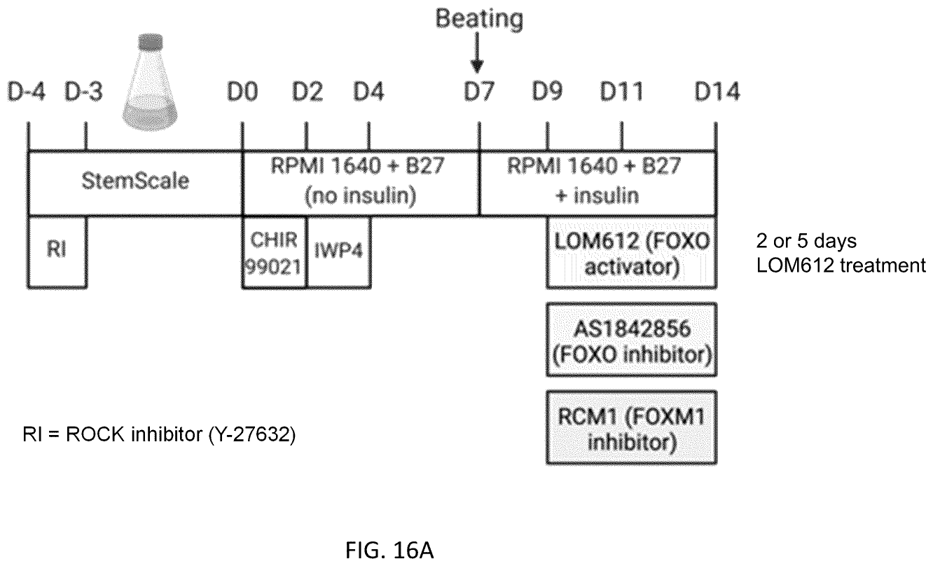

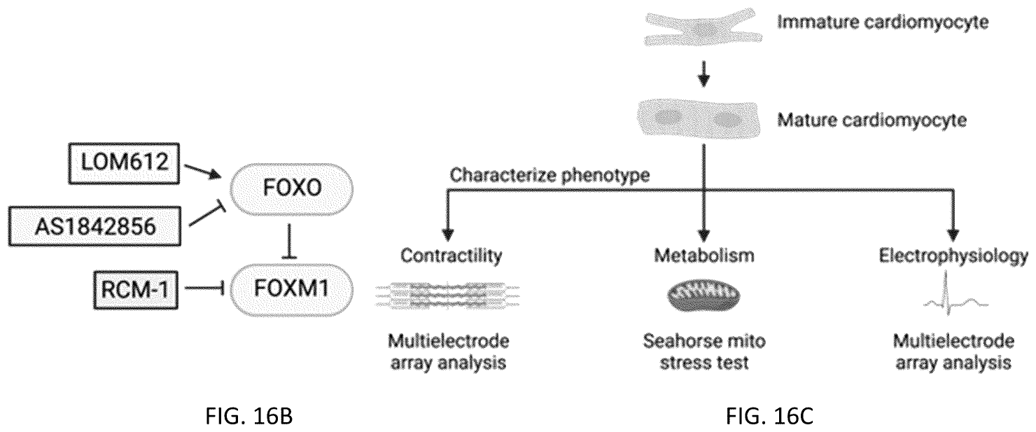

Cell lines used included UCSD142i-86-1 iPSC line (WiCell), Gibco human episomal iPSC line (Thermo), and DiPS 1016SevA iPSC line (Harvard. Cardiomyocytes are obtained utilizing a differential protocol adapted from Lian et al. Nat Protoc 2013: 8 (1): 162-75 (FIG. 16A). The differentiation protocol may be modified utilizing modulators of FOXO and FOXM1 (FIG. 16B). Cardiomyocytes may be characterized as being contractility mature, metabolically mature, or electrophysiologically mature (FIG. 16C).

### Results

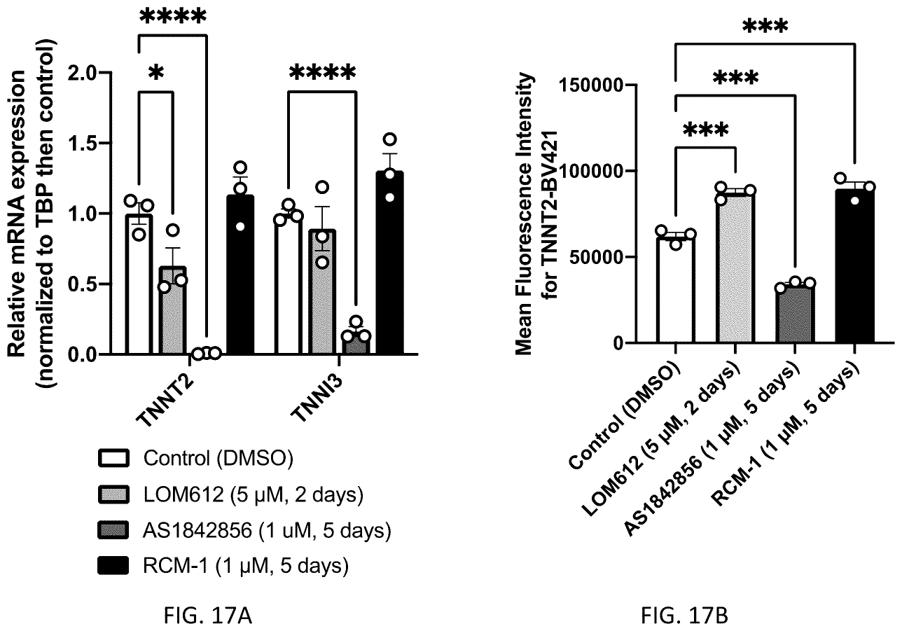

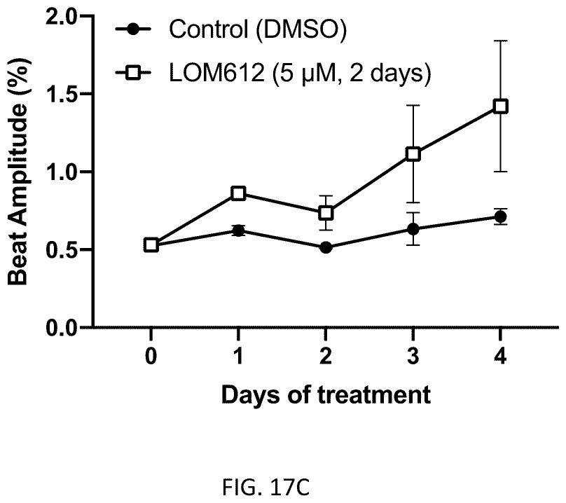

The contractile properties of cardiomyocytes improved with FOXO activation. FOXO activation with LOM612 or FOXM1 inhibition with RCM-1 increases protein expression of cardiac troponin T (TNNT2) and increases mean beat amplitude by multielectrode array analysis (FIG. 17). FOXO inhibition with AS1842856 results in non-beating spheroids with a significant decrease in cardiac troponin T and I expression. AS1842856 may be applied in an amount of about 0.2 to 1 μM.

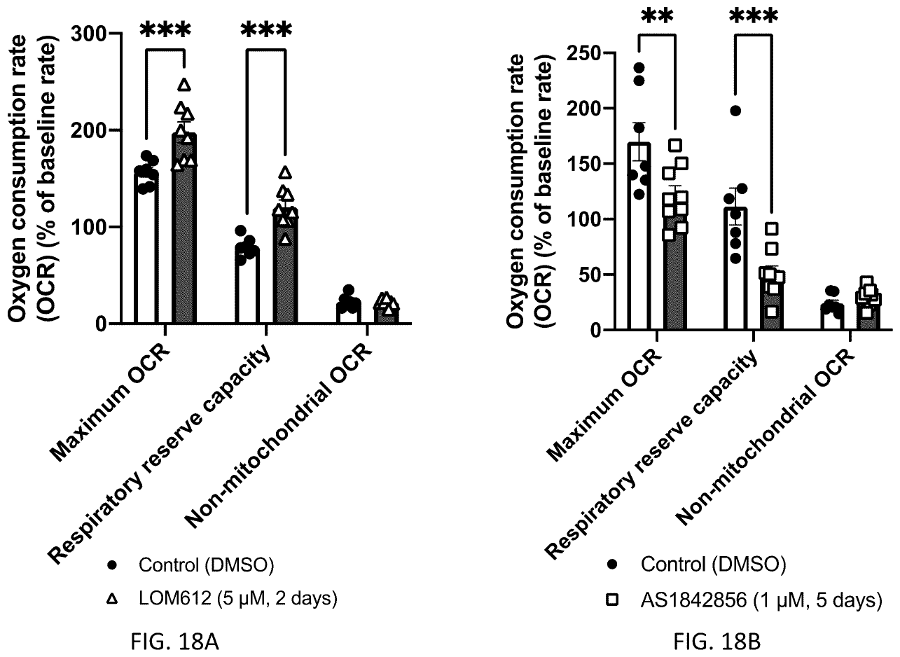

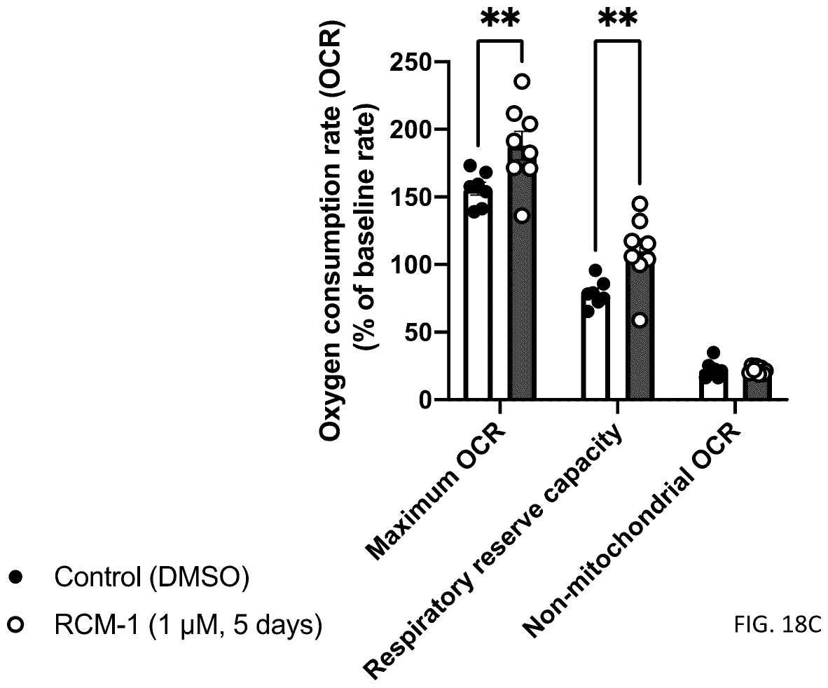

FOXO activation was shown to promote metabolic maturation of PSC-CMs. FOXO activation with LOM612 or FOXM1 inhibition with RCM-1 increases maximum oxygen consumption rate (OCR) and respiratory reserve capacity, while inhibition of FOXO with AS1842856 inhibits maximum OCR and respiratory reserve capacity (FIG. 18).

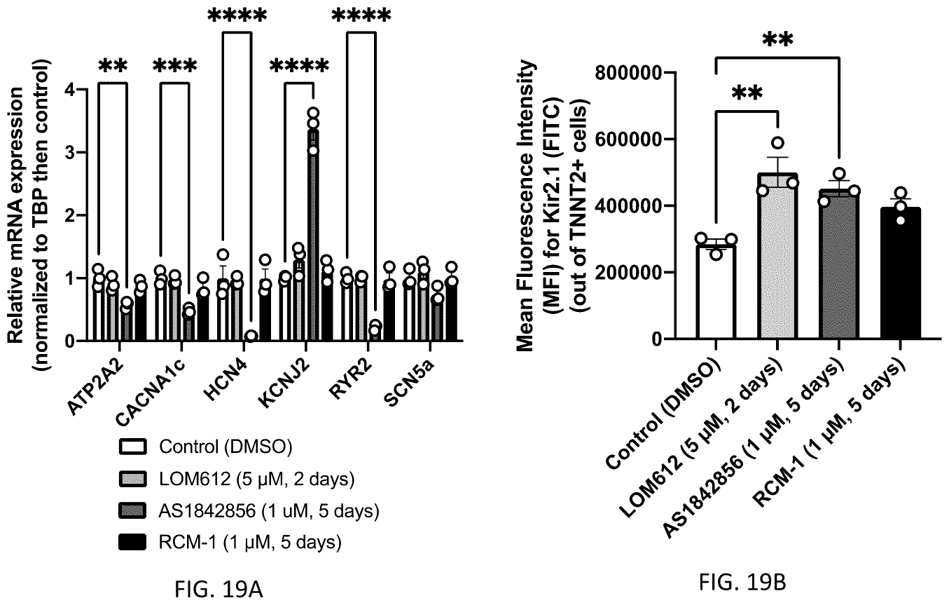

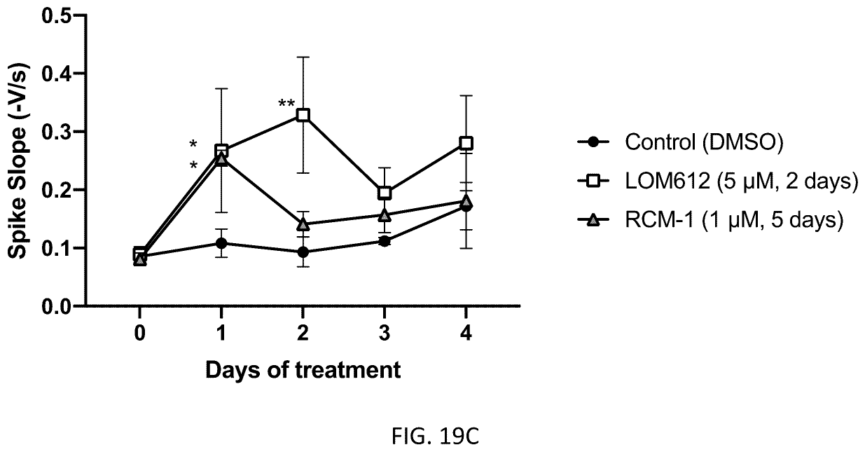

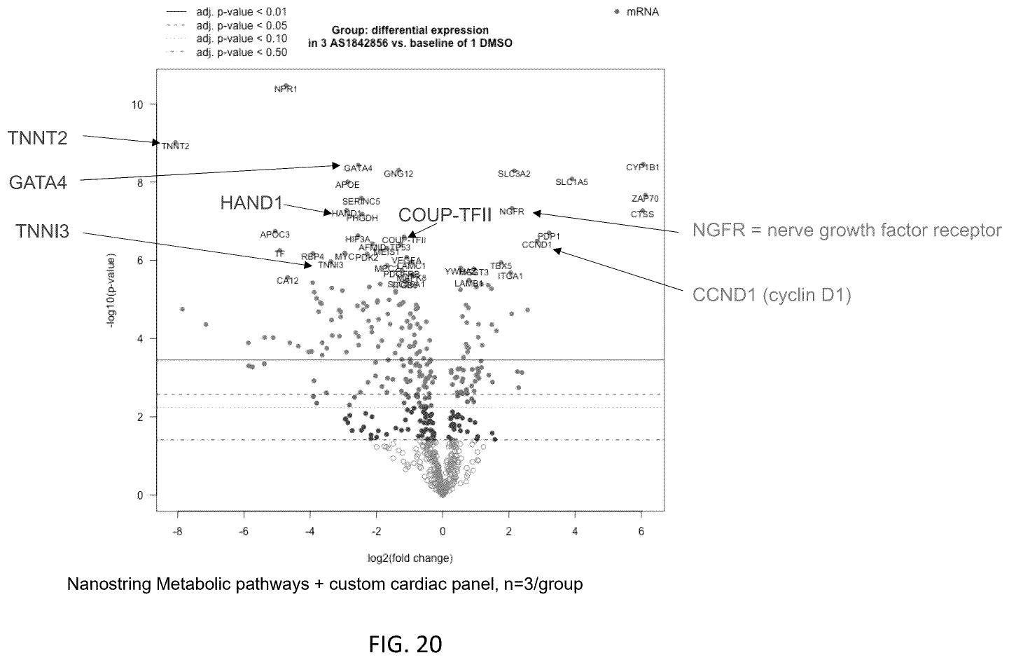

FOXO activation improved electrophysiological properties of PSC-CMs. FOXO activation with LOM612 enhances protein, but not RNA expression of Kir2.1 (FIGS. 19A-19B). FOXO activation increases the upstroke velocity (spike slope) (FIG. 19C). FOXO inhibition with AS1842856 was shown to reduce cardiac marker RNA levels (FIG. 20).

### Conclusions

FOXO activation with LOM612 enhances contractility and improves maturation of electrophysiological and metabolic parameters in iPSC-derived cardiomyocytes. FOXO inhibition with AS1842856 generates poorly contractile cells with a significant decrease in cardiac marker expression, suggesting a shift away from a cardiomyocyte phenotype.

1. A method of producing a human mature cardiomyocyte comprising co-culturing a human immature cardiomyocyte with a human endothelial cell.

2. The method of claim 1, wherein the immature cardiomyocyte is an iPSC-derived cardiomyocyte and/or wherein the endothelial cell is an iPSC-derived endothelial cell.

3. (canceled)

4. The method of claim 2, further comprising culturing the iPSC-derived cardiomyocyte and the iPSC-derived endothelial cell with at least one cardiomyocyte maturation factor selected from the group consisting of an mTOR signaling pathway inhibitor, a p53 upregulator, a FOXO activator, a FOXM1 inhibitor, and combinations thereof.

5. (canceled)

6. (canceled)

7. (canceled)

8. The method of claim 1, wherein the mature cardiomyocyte exhibits increased expression of a marker selected from the group consisting of cardiac troponin T (TNNT2), cardiac troponin I (TNNI3), Kir2.1, connexin 43 (Cx43), CD36, and combinations thereof as compared to an immature cardiomyocyte; and/or
wherein the mature cardiomyocyte exhibits increased respiratory reserve capacity as compared to an immature cardiomyocyte; and/or
wherein the mature cardiomyocyte exhibits decreased activity of an action selected from the group consisting of beating rate, automaticity, and combinations thereof as compared to an immature cardiomyocyte; and/or
wherein the mature cardiomyocyte is an electrically mature cardiomyocyte; and/or
wherein the mature cardiomyocyte exhibits a decreased risk of arrhythmia after delivery in vivo as compared to an immature cardiomyocyte; and/or
wherein the mature cardiomyocyte exhibits improved engraftment in vivo as compared to an immature cardiomyocyte.

9.-21. (canceled)

22. The method of claim 1, wherein the co-culturing occurs in three-dimensional culture and/or the co-culturing occurs in vitro or in vivo.

23. (canceled)

24. (canceled)

25. A non-naturally occurring cardiomyocyte produced by the methods of claim 1.

26.-32. (canceled)

33. A method of treatment comprising administering to a subject in need thereof a composition comprising at least one mature cardiomyocyte produced by the methods of claim 1.

34. The method of claim 33, wherein the subject has, or is at risk of developing, a ventricular arrhythmia, decreased systolic heart function, chronic heart failure, congenital heart disease, or other heart disease.

35. Use of a composition in the manufacture of a medicament for treatment of a heart condition, wherein the treatment comprises administration of the medicament to a subject in need thereof, wherein the composition comprises at least one mature cardiomyocyte produced by the methods of claim 1.

36. (canceled)

37. A three-dimensional structure comprising the mature cardiomyocytes produced by the methods of claim 1.

38. The three-dimensional structure of claim 37, wherein the three-dimensional structure is a matrix or scaffold and/or wherein the three-dimensional structure is administered to a subject.

39. (canceled)

40. A method of producing a mature cardiomyocyte comprising co-culturing an iPS-derived immature cardiomyocyte with an iPS-derived endothelial cell.

41. The method of claim 40, wherein the iPS-derived immature cardiomyocyte comprises a human cardiomyocyte and/or the iPS-derived endothelial cell comprises a human endothelial cell.

42. (canceled)

43. The method of claim 40, further comprising culturing the iPS-derived cardiomyocyte and the iPS-derived endothelial cell with at least one cardiomyocyte maturation factor selected from the group consisting of an mTOR signaling pathway inhibitor, a p53 upregulator, a FOXO activator, a FOXM1 inhibitor, and combinations thereof.

44. (canceled)

45. (canceled)

46. (canceled)

47. The method of claim 40, wherein the mature cardiomyocyte exhibits increased expression of cardiac troponin T (TNNT2), cardiac troponin I (TNNI3), Kir2.1, connexin 43 (Cx43), CD36, and combinations thereof as compared to an immature cardiomyocyte; and/or
wherein the mature cardiomyocyte exhibits decreased activity of an action selected from the group consisting of beating rate, automaticity, and combinations thereof as compared to an immature cardiomyocyte; and/or
wherein the mature cardiomyocyte is an electrically mature cardiomyocyte; and/or
wherein the mature cardiomyocyte exhibits a decreased risk of arrhythmia after delivery in vivo as compared to an immature cardiomyocyte; and/or
wherein the mature cardiomyocyte exhibits improved engraftment in vivo as compared to an immature cardiomyocyte.

48.-62. (canceled)

63. A non-naturally occurring cardiomyocyte produced by the methods of claim 40.

64.-70. (canceled)

71. A method of treatment comprising administering to a subject in need thereof a composition comprising at least one mature cardiomyocyte produced by the methods of claim 40.

72.-77. (canceled)

78. A method of producing a mature cardiomyocyte from an immature cardiomyocyte comprising contacting the immature cardiomyocyte with at least one cardiomyocyte maturation factor selected from the group consisting of FOXO activator, FOXM1 inhibitor, and combinations thereof.

79. The method of claim 78, wherein the at least one cardiomyocyte maturation factor is selected from the group consisting of Torin2, LOM612, metformin, resveratrol, Selinexor, celecoxib, doxorubicin, carbenoxolone, RCM1, FDI-6, thiostrepton, honokiol, siomycin A, curcumin, bortezomib, MG115, MG132, and combinations thereof.

80.-101. (canceled)

102. A non-naturally occurring cardiomyocyte produced by the methods of claim 78.

103.-113. (canceled)
